(19) United States
(12) Patent Application Publication
(10) Pub. No.: US 2010/0081129 A1
(43) Pub. Date: Apr. 1, 2010

(54) Genemap of the human genes associated with crohn's disease

(76) Inventors: Abdelmajid Belouchi, Ville St-Laurent (CA); John Verner Raelson, Hudson (CA); Walter Edward Bradley, Montreal (CA); Bruno Paquin, Chateauguay (CA); Hélene Fournier, Montreal (CA); Quynh Nguyen-Huu, Longveuil (CA); Pascal Croteau, Laval (CA); Réne Allard, Montreal (CA); Sophie Debrus, Montreal (CA); Valerie Serre, Kirkland (CA); Paul Van Eerdewegh, Carlisle, MA (US); Randall David Little, Laval (CA); Jonathan Segal, Efrat (IL); Tim Keith, Bedford, MA (US)

Correspondence Address: DOWELL & DOWELL P.C., 103 Oronoco St., Suite 220, Alexandria, VA, 22314, US

(21) Appl. No.: 11/919,503

(22) PCT Filed: May 1, 2006
(86) PCT No.: PCT/CA2006/000703

§ 371 Date: Sep. 24, 2009

Related U.S. Application Data
(60) Provisional application No. 60/675,841, filed on Apr. 29, 2005.

(51) Int. Cl. C12Q 1/68

(52) U.S. Cl. 435/6

(57) ABSTRACT

The present invention relates to the selection of a set of polymorphism markers for use in genome wide association studies based on linkage disequilibrium mapping. In particular, the invention relates to the fields of pharmacogenomics, diagnostics, patient therapy and the use of genetic haplotype information to predict an individual's susceptibility to Crohn's disease and/or their response to a particular drug or drugs.

### CROSS REFERENCE TO RELATED APPLICATIONS

This application is entitled to priority pursuant to 35 U.S.C. §119(e) to U.S. provisional patent application No. 60/675,841, which was filed on Apr. 29, 2005, which is incorporated herein in its entirety.

### FIELD OF THE INVENTION

The invention relates to the field of genomics and genetics, including genome analysis and the study of DNA variations. In particular, the invention relates to the fields of pharmacogenomics, diagnostics, patient therapy and the use of genetic haplotype information to predict an individual's susceptibility to Crohn's disease and/or their response to a particular drug or drugs, so that drugs tailored to genetic differences of population groups may be developed and/or administered to the appropriate population.

The invention also relates to a GeneMap for Crohn's disease, which links variations in DNA (including both genic and non-genic regions) to an individual's susceptibility to Crohn's disease and/or response to a particular drug or drugs. The invention further relates to the genes disclosed in the GeneMap (see Tables 8, 9, and 19-24), which is related to methods and reagents for detection of an individual's increased or decreased risk for Crohn's disease by identifying at least one polymorphism in one or a combination of the genes from the GeneMap. Also related are the candidate regions identified in Table 1, which are associated with Crohn's disease. In addition, the invention further relates to nucleotide sequences of those genes including genomic DNA sequences, cDNA sequences, single nucleotide polymorphisms (SNPs), other types of polymorphisms (insertions, deletions, microsatellites), alleles and haplotypes (see Sequence Listing and Tables 2-7, 11-18).

The invention further relates to isolated nucleic acids comprising these nucleotide sequences and isolated polypeptides or peptides encoded thereby. Also related are expression vectors and host cells comprising the disclosed nucleic acids or fragments thereof, as well as antibodies that bind to the encoded polypeptides or peptides.

The present invention further relates to ligands that modulate the activity of the disclosed genes or gene products. In addition, the invention relates to diagnostics and therapeutics for Crohn's disease, utilizing the disclosed nucleic acids, polymorphisms, chromosomal regions, gene maps, polypeptides or peptides, antibodies and/or ligands and small molecules that activate or repress relevant signaling events.

### BACKGROUND OF THE INVENTION

Crohn's disease is an Inflammatory Bowel Disease (IBD) in which inflammation extends beyond the inner gut lining and penetrates deeper layers of the intestinal wall of any part of the digestive system (esophagus, stomach, small intestine, large intestine, and/or anus). Crohn's disease is a chronic, lifelong disease which can cause painful, often life altering symptoms including diarrhea, cramping and rectal bleeding. Crohn's disease occurs most frequently in the industrialized world and the typical age of onset falls into two distinct ranges, 15 to 30 years of age and 60 to 80 years of age. The highest mortality is during the first years of disease, and in cases where the disease symptoms are long lasting, an increased risk of colon cancer is observed. Crohn's disease presently accounts for approximately two thirds of IBD-related physician visits and hospitalizations, and 50 to 80% of Crohn's disease patients eventually require surgical treatment. Development of Crohn's disease is influenced by environmental and host specific factors, together with “exogenous biological factors” such as constituents of the intestinal flora (the naturally occurring bacteria found in the intestine). It is believed that in genetically predisposed individuals, exogenous factors such as infectious agents, and host-specific characteristics such as intestinal barrier function and/or blood supply, combine with specific environmental factors to cause a chronic state of improperly regulated immune system function. In this hypothetical model, microorganisms trigger an immune response in the intestine, and in susceptible individuals, this immune response is not turned off when the microorganism is cleared from the body. The chronically “turned on” immune response causes damage to the intestine resulting in the symptoms of Crohn's disease.

Current treatments for Crohn's disease are primarily aimed at reducing symptoms by, suppressing inflammation and do not address the root cause of the disease. Despite a preponderance of evidence showing inheritance of a risk for Crohn's disease through epidemiological studies and genome wide linkage analyses, the genes affecting Crohn's disease have yet to be discovered (Hugot J P, and Thomas G., 1998). There is a need in the art for identifying specific genes related to Crohn's disease to enable the development of therapeutics that address the causes of the disease rather than relieving its symptoms. The failure in past studies to identify causative genes in complex diseases, such as Crohn's disease, has been due to the lack of appropriate methods to detect a sufficient number of variations in genomic DNA samples (markers), the insufficient quantity of necessary markers available, and the number of needed individuals to enable such a study. The present invention addresses these issues.

The DNA sequences between two human genomes are 99.9% identical. The variations in DNA sequence between individuals can be, as an example, deletions of small or large stretches of DNA, insertions of stretches of DNA, variations in the number of repetitive DNA elements, and changes in single base positions in the genome called “single nucleotide polymorphisms” (SNPs). Human DNA sequence variation accounts for a large fraction of observed differences between individuals, including susceptibility to disease.

Many common diseases, like Crohn's disease, are complex genetic traits and are believed to involve several disease-genes rather than single genes, as is observed for rare diseases. This makes detection of any particular gene substantially more difficult than in a rare disease, where a single gene mutation that segregates according to a Mendelian inheritance pattern is the causative mutation. Any one of the multiple interacting gene mutations involved in the etiology of a complex disease will impart a lower relative risk for the disease than will the single gene mutation involved in a simple genetic disease. Low relative risk alleles are more difficult to detect and, as a result, the success of positional cloning using linkage mapping that was achieved for simple genetic disease genes has not been repeated for complex diseases.

Several approaches have been proposed to discover and characterize multiple genes in complex genetic traits. These gene discovery methods can be subdivided into hypothesis-free disorder association studies and hypothesis-driven candidate gene or region studies. The candidate gene approach relies on the analysis of a gene in patients who have a disorder in which the gene is thought to play a role. This approach is limited in utility because it only provides for the investigation of genes with known functions. Although variant sequences of candidate genes may be identified using this approach, it is inherently limited by the fact that variant sequences in other genes that contribute to the phenotype will be necessarily missed when the technique is employed. Genome-wide scans (GWS) have been shown to be efficient in identifying Crohn's disease susceptibility genes (NOD2/CARD15 and OCTN). In contrast to the candidate gene approach, a GWS searches throughout the genome without any a priori hypothesis and consequently can identify genes that are not obvious candidates for the disease as well as genes that are relevant candidates for the disease it can also identify chromosomal regions that are structurally important where mutations can influence gene function of specific genes.

Family-based linkage mapping methods were initially used for disorder locus identification. This technique locates genes based on the relatively limited number of genetic recombination events within the families used in the study, and results in large chromosomal regions containing hundreds of genes, any one of which could be the disorder-causing gene. Population-based, or linkage disequilibrium (LD) mapping is based on the premise that regions adjacent to a gene of interest are co-transmitted through the generations along with the gene. As a result, LD extends over shorter genetic regions than does linkage (Hewett et al., 2002), and can facilitate detection of genes with lower relative risk than family linkage mapping approaches. LD-based mapping also defines much smaller candidate regions which may contain only a few genes, making the identification of the actual disorder gene much easier.

It has been estimated that a GWS that uses a general population and case/control association (LD) analysis would require approximately 700,000 SNP markers (Carlson et al., 2003). The cost of a GWS at this marker density for a sufficient sample size for statistical power is economically prohibitive. The use of a special founder population (genetic isolate), such as the French Canadian population of Quebec, is one solution to the problem with LD analysis. The French Canadian population in Quebec (Quebec Founder Population—QFP) provides one of the best resources in the world for gene discovery based on its high levels of genetic sharing and genetic homogeneity. By combining DNA collected from the QFP, high throughput genotyping capabilities and proprietary algorithms for genetic analysis, a comprehensive genome-wide association study was facilitated. The present invention relates specifically to a set of Crohn's disease-causing genes (GeneMap) and targets which present attractive points of therapeutic intervention.

In view of the foregoing, identifying susceptibility genes associated with Crohn's disease and their respective biochemical pathways will facilitate the identification of diagnostic markers as well as novel targets for improved therapeutics. It will also improve the quality of life for those afflicted by this disease and will reduce the economic costs of these afflictions at the individual and societal level. The identification of those genetic markers would provide the basis for novel genetic tests and eliminate or reduce the therapeutic methods currently used. The identification of those genetic markers will also provide the development of effective therapeutic intervention for the battery of laboratory, radiological, and endoscopic evaluations typically required to diagnose Crohn's disease. The present invention satisfies this need and provides related advantages as well.

### BRIEF DESCRIPTION OF THE DRAWINGS

FIGS. 1 to 8: Graphical representation of networks 1 to 23. Lists of directly interacting genes, as described in the text, were imported in the IPA software from Ingenuity Systems Inc. to generate networks of interacting genes (see Table 19 for the list of genes imported). These networks are based on functional relationships between gene products using known interactions in the literature. For each network, some nodes were manually extended to include good candidate genes that could play a role in the biochemical pathways of Crohn's disease. See Table 20 for a summary of the networks generated.

FIGS. 9-16: Graphical representation of networks 1b to 17b. Lists of directly and indirectly interacting genes, as described in the text, were imported in the IPA software from Ingenuity Systems Inc. to generate networks of interacting genes (see Table 19 for the list of genes imported). These networks are based on functional relationships between gene products using known interactions and relationships in the literature. For each network, some nodes were manually extended to include good candidate genes that could play a role in the biochemical pathways of Crohn's disease. See Table 21 for a summary of the networks generated.

FIGS. 17-19: Graphical representation of networks 1-12, based on a subset of the candidate genes identified. A list of 61 genes, as described in the text, was imported in the IPA software from Ingenuity Systems Inc. to generate networks of directly interacting genes (see Table 22 for the list of genes imported). These networks are based on functional relationships between gene products using known interactions and relationships in the literature. For each network, some nodes were manually extended to include good candidate genes that could play a role in the biochemical pathways of Crohn's disease. See Table 23 for a summary of the networks generated.

FIGS. 20-23: Graphical representation of networks 1-4, based on a subset of the candidate genes identified. A list of 61 genes, as described in the text, was imported in the IPA software from Ingenuity Systems Inc. to generate networks of direct and indirect gene interactions (see Table 22 for the list of genes imported). These networks are based on functional relationships between gene products using known interactions and relationships in the literature. For each network, some nodes were manually extended to include good candidate genes that could play a role in the biochemical pathways of Crohn's disease. See Table 24 for a summary of the networks generated.

### DESCRIPTION OF THE FILES CONTAINED ON THE CD-R

The CD-R contains an electronic copy of the sequence listing and Tables 1-24 related thereto.

### DEFINITIONS

Throughout the description of the present invention, several terms are used that are specific to the science of this field. For the sake of clarity and to avoid any misunderstanding, these definitions are provided to aid in the understanding of the specification and claims:

Allele: One of a pair, or series, of forms of a gene or non-genic region that occur at a given locus in a chromosome. Alleles are symbolized with the same basic symbol (e.g., B for dominant and b for recessive; B1, B2, Bn for n additive alleles at a locus). In a normal diploid cell there are two alleles of any one gene (one from each parent), which occupy the same relative position (locus) on homologous chromosomes. Within a population there may be more than two alleles of a gene. See multiple alleles. SNPs also have alleles, i.e., the two (or more) nucleotides that characterize the SNP.

Amplification of nucleic acids: refers to methods such as polymerase chain reaction (PCR), ligation amplification (or ligase chain reaction, LCR) and amplification methods based on the use of Q-beta replicase. These methods are well known in the art and are described, for example, in U.S. Pat. Nos. 4,683,195 and 4,683,202. Reagents and hardware for conducting PCR are commercially available. Primers useful for amplifying sequences from the disorder region are preferably complementary to, and preferably hybridize specifically to, sequences in the disorder region or in regions that flank a target region therein. Genes from Tables 8, 9, 19, 20, 21, 22, 23 or 24 generated by amplification may be sequenced directly. Alternatively, the amplified sequence(s) may be cloned prior to sequence analysis.

Antigenic component: is a moiety that binds to its specific antibody with sufficiently high affinity to form a detectable antigen-antibody complex.

Antibodies: refer to polyclonal and/or monoclonal antibodies and fragments thereof, and immunologic binding equivalents thereof, that can bind to proteins and fragments thereof or to nucleic acid sequences from the disorder region, particularly from the disorder gene products or a portion thereof. The term antibody is used both to refer to a homogeneous molecular entity, or a mixture such as a serum product made up of a plurality of different molecular entities. Proteins may be prepared synthetically in a protein synthesizer and coupled to a carrier molecule and injected over several months into rabbits. Rabbit sera are tested for immunoreactivity to the protein or fragment. Monoclonal antibodies may be made by injecting mice with the proteins, or fragments thereof. Monoclonal antibodies can be screened by ELISA and tested for specific immunoreactivity with protein or fragments thereof (Harlow et al. 1988, Antibodies: A Laboratory Manual, Cold Spring Harbor Laboratory, Cold Spring Harbor, N.Y.). These antibodies will be useful in developing assays as well as therapeutics.

Associated allele: refers to an allele at a polymorphic locus that is associated with a particular phenotype of interest, e.g., a predisposition to a disorder or a particular drug response.

cDNA: refers to complementary or copy DNA produced from an RNA template by the action of RNA-dependent DNA polymerase (reverse transcriptase). Thus, a cDNA clone means a duplex DNA sequence complementary to an RNA molecule of interest, included in a cloning vector or PCR amplified. This term includes genes from which the intervening sequences have been removed.

cDNA library: refers to a collection of recombinant DNA molecules containing cDNA inserts that together comprise essentially all of the expressed genes of an organism or tissue. A cDNA library can be prepared by methods known to one skilled in the art (see, e.g., Cowell and Austin, 1997, “DNA Library Protocols,” Methods in Molecular Biology). Generally, RNA is first isolated from the cells of the desired organism, and the RNA is used to prepare cDNA molecules.

Cloning: refers to the use of recombinant DNA techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.

Cloning vector: refers to a plasmid or phage DNA or other DNA molecule that is able to replicate in a host cell. The cloning vector is typically characterized by one or more endonuclease recognition sites at which such DNA sequences may be cleaved in a determinable fashion without loss of an essential biological function of the DNA, and which may contain a selectable marker suitable for use in the identification of cells containing the vector.

Coding sequence or a protein-coding sequence: is a polynucleotide sequence capable of being transcribed into mRNA and/or capable of being translated into a polypeptide or peptide. The boundaries of the coding sequence are typically determined by a translation start codon at the 5′-terminus and a translation stop codon at the 3′-terminus.

Complement of a nucleic acid sequence: refers to the antisense sequence that participates in Watson-Crick base-pairing with the original sequence.

Disorder region: refers to the portions of the human chromosomes displayed in Table 1 bounded by the markers from Tables 2, 3, 4, 5, 6, 7 or 10.

Disorder-associated nucleic acid or polypeptide sequence: refers to a nucleic acid sequence that maps to region of Table 1 or the polypeptides encoded therein (Tables 8, 9, 19, 20, 21, 22, 23 or 24, nucleic acids, and polypeptides). For nucleic acids, this encompasses sequences that are identical or complementary to the gene sequences from Tables 8, 9, 19, 20, 21, 22, 23 or 24, as well as sequence-conservative, function-conservative, and non-conservative variants thereof. For polypeptides, this encompasses sequences that are identical to the polypeptide, as well as function-conservative and non-conservative variants thereof. Included are the alleles of naturally-occurring polymorphisms causative of Crohn's disease such as, but not limited to, alleles that cause altered expression of genes of Tables 8, 9, 19, 20, 21, 22, 23 or 24 and alleles that cause altered protein levels or stability (e.g., decreased levels, increased levels, expression in an inappropriate tissue type, increased stability, and decreased stability).

Expression vector: refers to a vehicle or plasmid that is capable of expressing a gene that has been cloned into it, after transformation or integration in a host cell. The cloned gene is usually placed under the control of (i.e., operably linked to) a regulatory sequence.

Function-conservative variants: are those in which a change in one or more nucleotides in a given codon position results in a polypeptide sequence in which a given amino acid residue in the polypeptide has been replaced by a conservative amino acid substitution. Function-conservative variants also include analogs of a given polypeptide and any polypeptides that have the ability to elicit antibodies specific to a designated polypeptide.

Founder population: Also called a population isolate, this is a large number of people who have mostly descended, in genetic isolation from other populations, from a much smaller number of people who lived many generations ago.

Gene: Refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions, as well as regulatory regions, and can include 5′ and 3′ ends. A gene sequence is wild-type if such sequence is usually found in individuals unaffected by the disorder or condition of interest. However, environmental factors and other genes can also play an important role in the ultimate determination of the disorder. In the context of complex disorders involving multiple genes (oligogenic disorder), the wild type, or normal sequence can also be associated with a measurable risk or susceptibility, receiving its reference status based on its frequency in the general population.

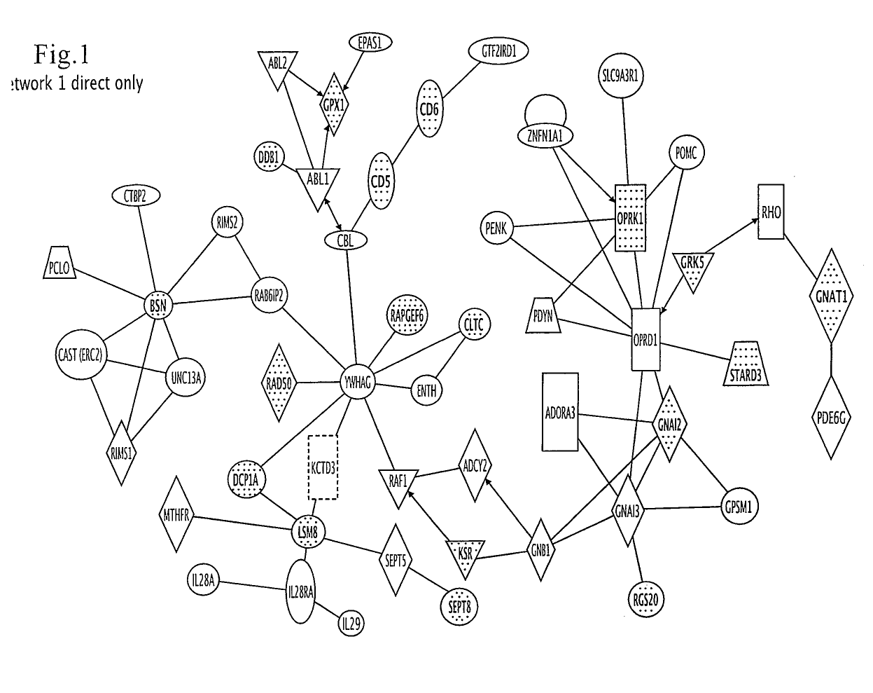

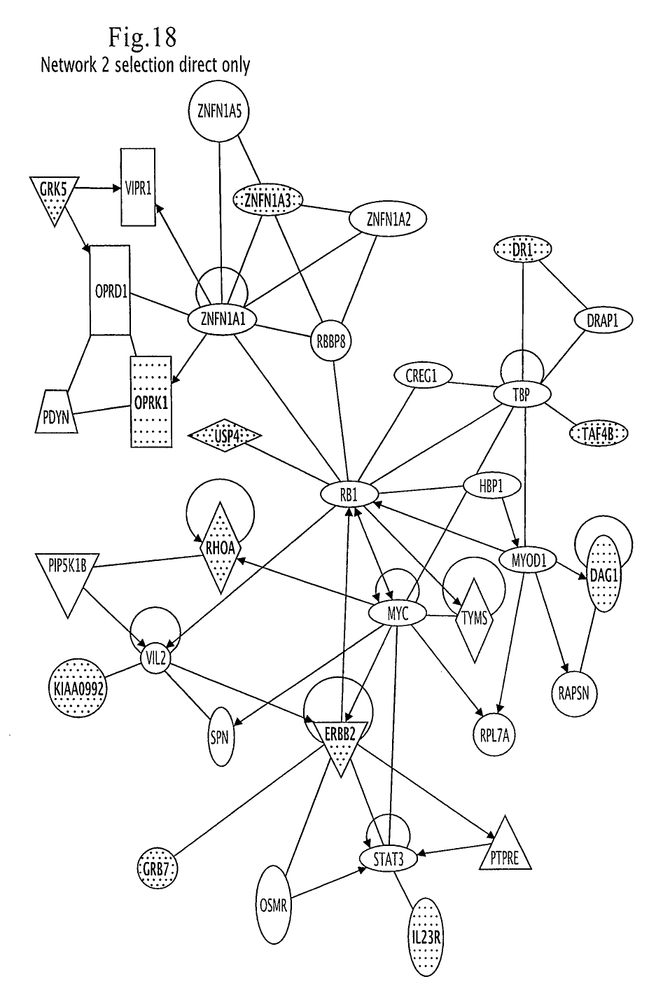

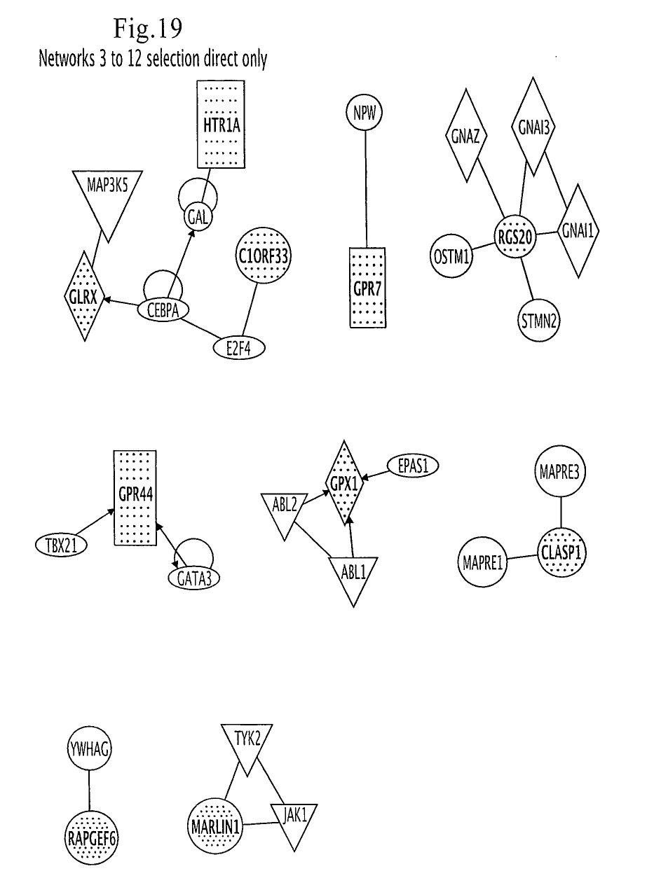

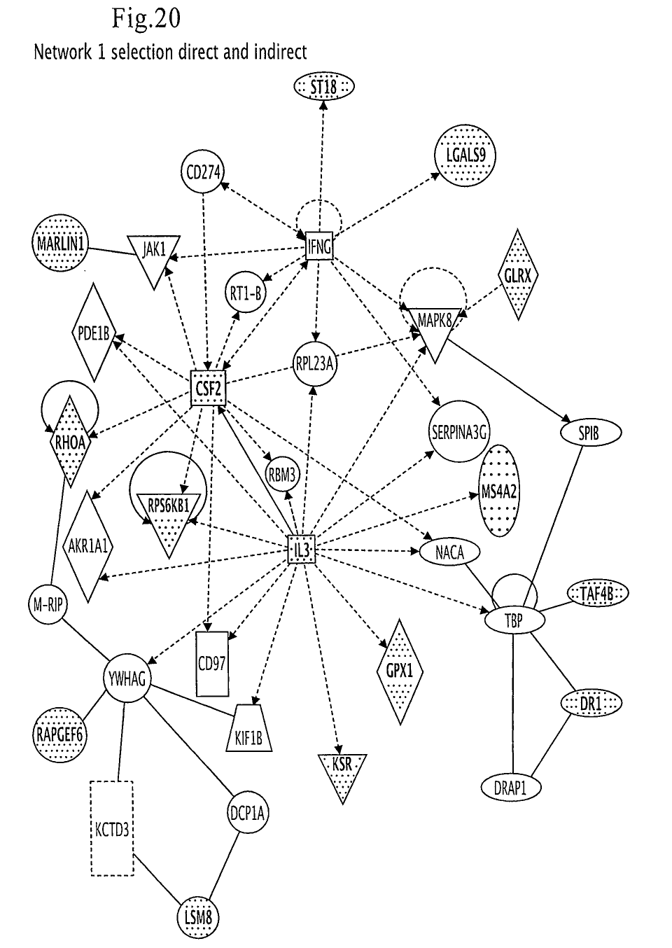

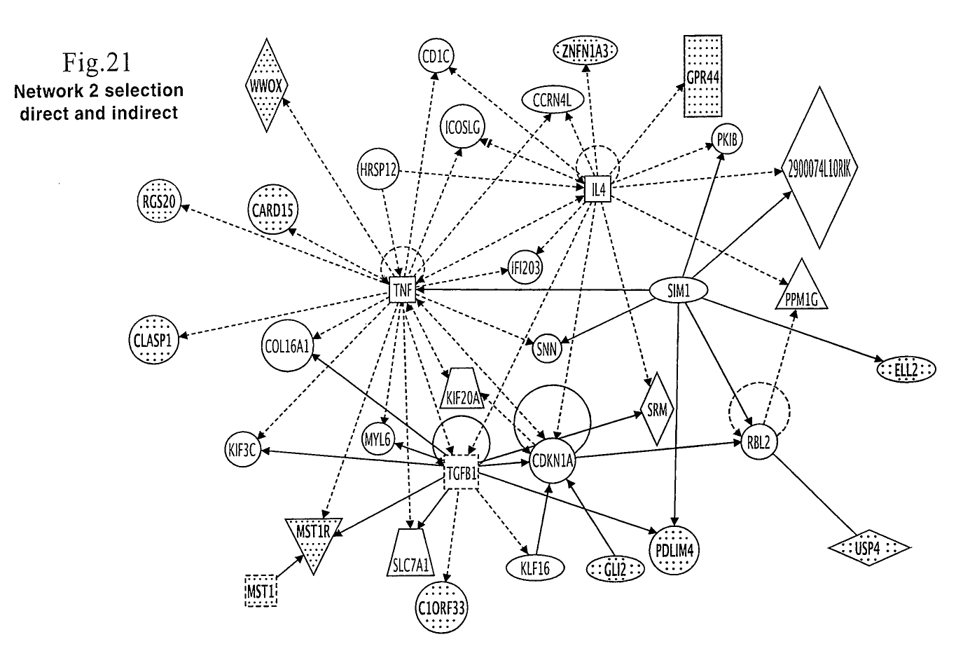

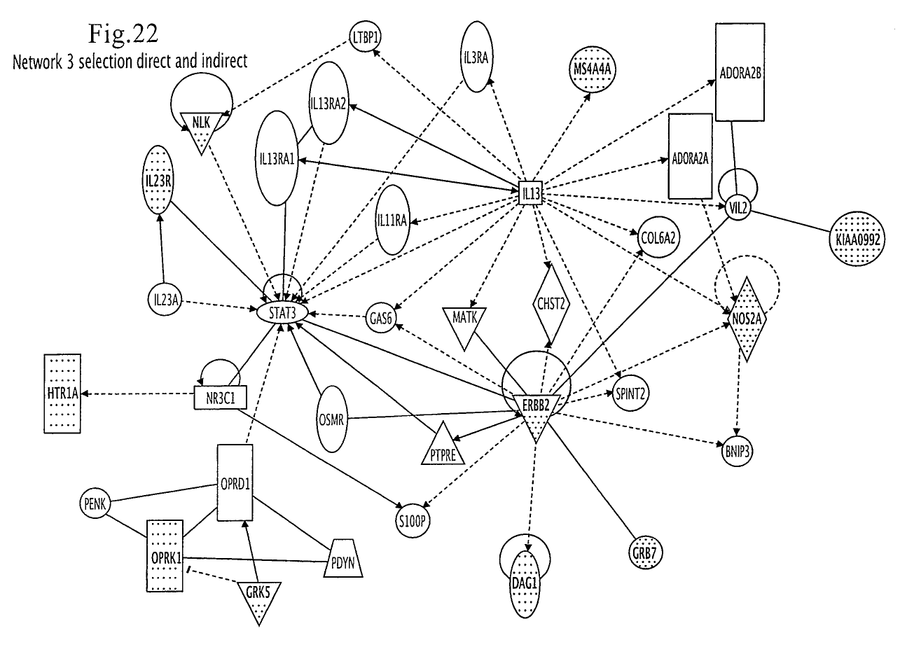

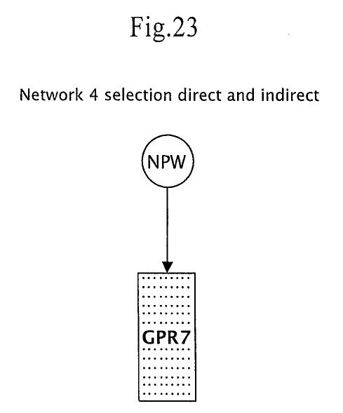

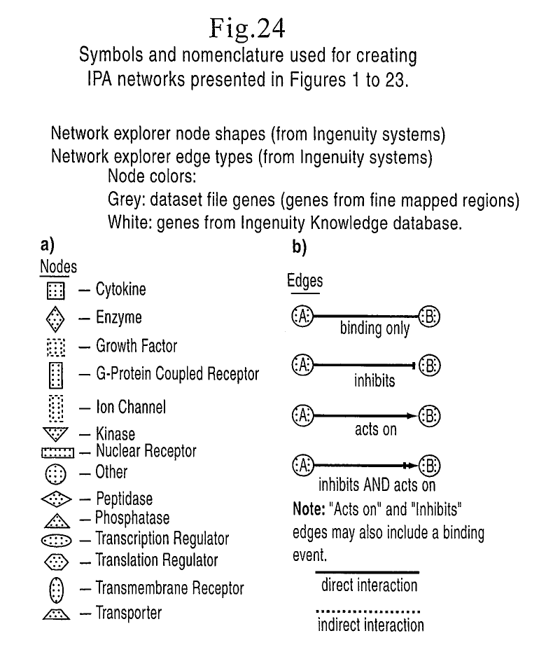

GeneMaps: are defined as groups of gene(s) that are directly or indirectly involved in at least one phenotype of a disorder (some non-limiting example, of GeneMaps comprises networks displayed in FIGS. 1 to 23 herein). As such, GeneMaps enable the development of synergistic diagnostic products, creating “theranostics”.

Genotype: Set of alleles at a specified locus or loci.

Haplotype: The allelic pattern of a group of (usually contiguous) DNA markers or other polymorphic loci along an individual chromosome or double helical DNA segment. Haplotypes identify individual chromosomes or chromosome segments. The presence of shared haplotype patterns among a group of individuals implies that the locus defined by the haplotype has been inherited, identical by descent (IBD), from a common ancestor. Detection of identical by descent haplotypes is the basis of linkage disequilibrium (LD) mapping. Haplotypes are broken down through the generations by recombination and mutation. In some instances, a specific allele or haplotype may be associated with susceptibility to a disorder or condition of interest, e.g., Crohn's disease. In other instances, an allele or haplotype may be associated with a decrease in susceptibility to a disorder or condition of interest, i.e., a protective sequence.

Host: includes prokaryotes and eukaryotes. The term includes an organism or cell that is the recipient of an expression vector (e.g., autonomously replicating or integrating vector).

Hybridizable: nucleic acids are hybridizable to each other when at least one strand of the nucleic acid can anneal to another nucleic acid strand under defined stringency conditions. In some embodiments, hybridization requires that the two nucleic acids contain at least 10 substantially complementary nucleotides; depending on the stringency of hybridization, however, mismatches may be tolerated. The appropriate stringency for hybridizing nucleic acids depends on the length of the nucleic acids and the degree of complementarity, and can be determined in accordance with the methods described herein.

Identity by descent (IBD): Identity among DNA sequences for different individuals that is due to the fact that they have all been inherited from a common ancestor. LD mapping identifies IBD haplotypes as the likely location of disorder genes shared by a group of patients.

Identity: as known in the art, is a relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity and similarity can be readily calculated by known methods, including but not limited to those described in A. M. Lesk (ed), 1988, Computational Molecular Biology, Oxford University Press, NY; D. W. Smith (ed), 1993, Biocomputing. Informatics and Genome Projects, Academic Press, NY; A. M. Griffin and H. G. Griffin, H. G (eds), 1994, ComputerAnalysis of Sequence Data, Part 1, Humana Press, NJ; G. von Heinje, 1987, Sequence Analysis in Molecular Biology, Academic Press; and M. Gribskov and J. Devereux (eds), 1991, Sequence Analysis Primer, M Stockton Press, NY; H. Carillo and D. Lipman, 1988, SIAM J. Applied Math., 48:1073.

Immunogenic component: is a moiety that is capable of eliciting a humoral and/or cellular immune response in a host animal.

Isolated nucleic acids: are nucleic acids separated away from other components (e.g., DNA, RNA, and protein) with which they are associated (e.g., as obtained from cells, chemical synthesis systems, or phage or nucleic acid libraries). Isolated nucleic acids are at least 60% free, preferably 75% free, and most preferably 90% free from other associated components. In accordance with the present invention, isolated nucleic acids can be obtained by methods described herein, or other established methods, including isolation from natural sources (e.g., cells, tissues, or organs), chemical synthesis, recombinant methods, combinations of recombinant and chemical methods, and library screening methods.

Isolated polypeptides or peptides: are those that are separated from other components (e.g., DNA, RNA, and other polypeptides or peptides) with which they are associated (e.g., as obtained from cells, translation systems, or chemical synthesis systems). In a preferred embodiment, isolated polypeptides or peptides are at least 10% pure; more preferably, 80% or 90% pure. Isolated polypeptides and peptides include those obtained by methods described herein, or other established methods, including isolation from natural sources (e.g., cells, tissues, or organs), chemical synthesis, recombinant methods, or combinations of recombinant and chemical methods. Proteins or polypeptides referred to herein as recombinant are proteins or polypeptides produced by the expression of recombinant nucleic acids. A portion as used herein with regard to a protein or polypeptide, refers to fragments of that protein or polypeptide. The fragments can range in size from 5 amino acid residues to all but one residue of the entire protein sequence. Thus, a portion or fragment can be at least 5, 5-50, 50-100, 100-200, 200-400, 400-800, or more consecutive amino acid residues of a protein or polypeptide.

Linkage disequilibrium (LD): the situation in which the alleles for two or more loci do not occur together in individuals sampled from a population at frequencies predicted by the product of their individual allele frequencies. In other words, markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait. The physical proximity of markers can be measured in family studies where it is called linkage or in population studies where it is called linkage disequilibrium.

LD mapping: population based gene mapping, which locates disorder genes by identifying regions of the genome where haplotypes or marker variation patterns are shared statistically more frequently among disorder patients compared to healthy controls. This method is based upon the assumption that many of the patients will have inherited an allele associated with the disorder from a common ancestor (IBD), and that this allele will be in LD with the disorder gene.

Locus: a specific position along a chromosome or DNA sequence. Depending upon context, a locus could be a gene, a marker, a chromosomal band or a specific sequence of one or more nucleotides.

Minor allele frequency (MAF): the population frequency of one of the alleles for a given polymorphism, which is equal or less than 50%. The sum of the MAF and the Major allele frequency equals one.

Markers: an identifiable DNA sequence that is variable (polymorphic) for different individuals within a population. These sequences facilitate the study of inheritance of a trait or a gene. Such markers are used in mapping the order of genes along chromosomes and in following the inheritance of particular genes; genes closely linked to the marker or in LD with the marker will generally be inherited with it. Two types of markers are commonly used in genetic analysis, microsatellites and SNPs.

Microsatellite: DNA of eukaryotic cells comprising a repetitive, short sequence of DNA that is present as tandem repeats and in highly variable copy number, flanked by sequences unique to that locus.

Mutant sequence: if it differs from one or more wild-type sequences. For example, a nucleic acid from a gene listed in Tables 8, 9, 19, 20, 21, 22, 23 or 24 containing a particular allele of a single nucleotide polymorphism may be a mutant sequence. In some cases, the individual carrying this allele has increased susceptibility toward the disorder or condition of interest. In other cases, the mutant sequence might also refer to an allele that decreases the susceptibility toward a disorder or condition of interest and thus acts in a protective manner. The term mutation may also be used to describe a specific allele of a polymorphic locus.

Non-conservative variants: are those in which a change in one or more nucleotides in a given codon position results in a polypeptide sequence in which a given amino acid residue in a polypeptide has been replaced by a non-conservative amino acid substitution. Non-conservative variants also include polypeptides comprising non-conservative amino acid substitutions.

Nucleic acid or polynucleotide: purine- and pyrimidine-containing polymers of any length, either polyribonucleotides or polydeoxyribonucleotide or mixed polyribo polydeoxyribonucleotides. This includes single-and double-stranded molecules, i.e., DNA-DNA, DNA-RNA and RNA-RNA hybrids, as well as protein nucleic acids (PNA) formed by conjugating bases to an amino acid backbone. This also includes nucleic acids containing modified bases.

Nucleotide: a nucleotide, the unit of a DNA molecule, is composed of a base, a 2′-deoxyribose and phosphate ester(s) attached at the 5′ carbon of the deoxyribose. For its incorporation in DNA, the nucleotide needs to possess three phosphate esters but it is converted into a monoester in the process.

Operably linked: means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

Ortholog: denotes a gene or polypeptide obtained from one species that has homology to an analogous gene or polypeptide from a different species.

Paralog: denotes a gene or polypeptide obtained from a given species that has homology to a distinct gene or polypeptide from that same species.

Phenotype: any visible, detectable or otherwise measurable property of an organism such as symptoms of, or susceptibility to, a disorder.

Polymorphism: occurrence of two or more alternative genomic sequences or alleles between or among different genomes or individuals at a single locus. A polymorphic site thus refers specifically to the locus at which the variation occurs. In some cases, an individual carrying a particular allele of a polymorphism has an increased or decreased susceptibility toward a disorder or condition of interest.

Portion and fragment: are synonymous. A portion as used with regard to a nucleic acid or polynucleotide refers to fragments of that nucleic acid or polynucleotide. The fragments can range in size from 8 nucleotides to all but one nucleotide of the entire gene sequence. Preferably, the fragments are at least about 8 to about 10 nucleotides in length; at least about 12 nucleotides in length; at least about 15 to about 20 nucleotides in length; at least about 25 nucleotides in length; or at least about 35 to about 55 nucleotides in length.

Probe or primer: refers to a nucleic acid or oligonucleotide that forms a hybrid structure with a sequence in a target region of a nucleic acid due to complementarity of the probe or primer sequence to at least one portion of the target region sequence.

Protein and polypeptide: are synonymous. Peptides are defined as fragments or portions of polypeptides, preferably fragments or portions having at least one functional activity (e.g., proteolysis, adhesion, fusion, antigenic, or intracellular activity) as the complete polypeptide sequence.

Recombinant nucleic acids: nucleic acids which have been produced by recombinant DNA methodology, including those nucleic acids that are generated by procedures which rely upon a method of artificial replication, such as the polymerase chain reaction (PCR) and/or cloning into a vector using restriction enzymes. Portions of recombinant nucleic acids which code for polypeptides can be identified and isolated by, for example, the method of M. Jasin et al., U.S. Pat. No. 4,952,501.

Regulatory sequence: refers to a nucleic acid sequence that controls or regulates expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Regulatory sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.

Sample: as used herein refers to a biological sample, such as, for example, tissue or fluid isolated from an individual or animal (including, without limitation, plasma, serum, cerebrospinal fluid, lymph, tears, nails, hair, saliva, milk, pus, and tissue exudates and secretions) or from in vitro cell culture-constituents, as well as samples obtained from, for example, a laboratory procedure.

Single nucleotide polymorphism (SNP): variation of a single nucleotide. This includes the replacement of one nucleotide by another and deletion or insertion of a single nucleotide. Typically, SNPs are biallelic markers although tri- and tetra-allelic markers also exist. For example, SNP A\C may comprise allele C or allele A (Tables 2, 3, 4, 5, 6, 7 or 10). Thus, a nucleic acid molecule comprising SNP A\C may include a C or A at the polymorphic position. For clarity purposes, an ambiguity code is used in Tables 2, 3, 4, 5, 6, 7 or 10 and the sequence listing, to represent the variations. For a combination of SNPs, the term “haplotype” is used, e.g. the genotype of the SNPs in a single DNA strand that are linked to one another. In certain embodiments, the term “haplotype” is used to describe a combination of SNP alleles, e.g., the alleles of the SNPs found together on a single DNA molecule. In specific embodiments, the SNPs in a haplotype are in linkage disequilibrium with one another.

Sequence-conservative: variants are those in which a change of one or more nucleotides in a given codon position results in no alteration in the amino acid encoded at that position (i.e., silent mutation).

Substantially homologous: a nucleic acid or fragment thereof is substantially homologous to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases. Alternatively, substantial homology exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90% (M. Kanehisa, 1984, NucL Acids Res. 11:203-213). The length of homology comparison, as described, may be over longer stretches, and in certain embodiments will often be over a stretch of at least 14 nucleotides, usually at least 20 nucleotides, more usually at least 24 nucleotides, typically at least 28 nucleotides, more typically at least 32 nucleotides, and preferably at least 36 or more nucleotides.

Wild-type gene from Tables 8, 9, 19, 20, 21, 22, 23 or 24: refers to the reference sequence. The wild-type gene sequences from Tables 8, 9, 19, 20, 21, 22, 23 or 24 used to identify the variants (polymorphisms, alleles, and haplotypes) described in detail herein.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. Standard reference works setting forth the general principles of recombinant DNA technology include J. Sambrook et al., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC Press, Boca Raton; M. J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes I and 11; M. J. Gait (ed), 1984, Oligonucleotide Synthesis; B. D. Hames and S. J. Higgins (eds), 1984, Nucleic Acid Hybridization; Quirke and Taylor (eds), 1991, PCR-A Practical Approach; Harries and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning, J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology. Tools for Genomic and Molecular Research, American Society for Microbiology, Washington, D.C. Standard reference works setting forth the general principles of immunology include S. Sell, 1996, Immunology, Immunopathology & Immunity, 5th Ed., Appleton & Lange, Publ., Stamford, Conn.; D. Male et al., 1996, Advanced Immunology, 3d Ed., Times Mirror Int'l Publishers Ltd., Publ., London; D. P. Stites and A. L Terr, 1991, Basic and Clinical Immunology, 7th Ed., Appleton & Lange, Publ., Norwalk, Conn.; and A. K. Abbas et al., 1991, Cellular and Molecular Immunology, W. B. Saunders Co., Publ., Philadelphia, Pa. Any suitable materials and/or methods known to those of skill can be utilized in carrying out the present invention; however, preferred materials and/or methods are described. Materials, reagents, and the like to which reference is made in the following description and examples are generally obtainable from commercial sources, and specific vendors are cited herein.

### DETAILED DESCRIPTION OF THE INVENTION

General Description of Crohn's Disease

Inflammatory Bowel Disease (IBD) is characterized by excessive and chronic inflammation at various sites in the gastro-intestinal tract. IBD describes two clinical conditions called Crohn's disease (CD) and ulcerative colitis (UC). CD and UC share many clinical and pathological characteristics but they also have some markedly different features. There is strong scientific support suggesting that the main pathological processes in these two diseases are distinct. This patent application will focus primarily on Crohn's disease.

The United Kingdom, northern Europe, and North America have been reported to have the highest incidence rates and prevalence for CD. In North America, prevalence for CD ranges between 0.03% and 0.2%. However, reports of increasing incidence and prevalence from other areas of the world have been published over the past 30 years (reviewed in Loftus 2004). CD may occur in people of all ages, but it is most commonly diagnosed in late adolescence and early adulthood (reviewed in Andres and Friedman 1999). Any part of the gastrointestinal tract can be affected in CD, from the mouth to the anus, and patches of inflammation occur, interspersed with healthy tissue.

Most CD patients experience characteristic periods of remission and flare-ups of the disease, often-requiring long-term medication, and/or hospitalization and surgery. The symptoms and complications of Crohn's disease differ, depending on what part of the intestinal tract is inflamed. The severity of the disease does not correlate directly with the extent of bowel involvement. It is the disease pattern that is most important in determining the disease course and the nature of the associated complications. Thus, CD can be subdivided into 3 types: predominantly inflammatory CD, non-perforating CD (presence of strictures), or perforating CD (presence of fistulas and/or abscesses) (reviewed in Andres and Friedman 1999).

CD symptoms include chronic diarrhea, abdominal pain, cramping, anorexia, and weight loss. Systemic features include fatigue, tachycardia and pyrexia. Chronic or acute blood loss in the bowel may result in anemia and even shock. The most common complication of CD is the presence of strictures (obstruction) of the intestine due to swelling and the formation of scar tissue. Another complication involves sores or ulcers within the intestinal tract. Sometimes these deep ulcers turn into tracts called fistulas that connect different parts of the intestine. These fistulas often become infected and occasionally form an abscess. Extra-intestinal inflammatory manifestations can occur in joints, eyes, skin, mouth, and liver in patients with either forms of IBD (reviewed in Andres and Friedman 1999). CD patients also carry several risk factors for the development of osteoporosis such as calcium and vitamin D deficiency, and corticosteroid use (Tremaine 2003). Patients with CD are also at increased risk of cancer of both the small and the large intestine (reviewed in Andres and Friedman 1999). CD is associated with an increased mortality rate relative to the general population and independent of whether the small intestine, large intestine, or both are affected. The excess of mortality is most notable in the first few years after diagnosis and is most often attributable to complications of CD, including colorectal cancer as well as other gastrointestinal complications (reviewed in Andres and Friedman 1999). CD is a lifelong disease that causes symptoms that may interfere with social activities, interpersonal relationships, and employment. Impairment relates to disease severity, pattern and side-effects of medication, the possibility of surgery, but also to age, other demographic factors and co-morbid medical conditions, including depression and anxiety (Irvine 2004).

There is no single definitive test for the diagnosis of CD. To determine the diagnosis, physicians evaluate a combination of information from the history and physical examination of a patient and from results of endoscopic, radiologic and histologic (blood and tissue) tests. Endoscopy with biopsy is the cornerstone for diagnosing and evaluating disease activity in CD. Radiology tests are used together with endoscopy to help evaluate the small bowel and look at the entire abdomen for infections, strictures, obstructions, and fistulas. Because CD often mimics other conditions and symptoms may vary widely, it may take some time to confirm the diagnosis.

Because there is no cure for CD, the goal of medical treatment is to suppress the inflammatory response and alleviate the symptoms by decreasing the frequency of disease flare-ups and maintaining remissions. Non-surgical treatment for active disease involves the use of anti-inflammatory (aminosalicylates and corticosteroids), antimicrobial (antibiotics), and immunomodulatory agents to control symptoms and reduce disease activity. The biologic therapies are targeted towards specific disease mechanisms and have the potential to provide more effective and safe treatments for human diseases. Infliximab (Remicade®) is a chimeric monoclonal antibody against TNFalpha, and the first biologic therapy that was approved for CD. Several novel genetically engineered drugs targeting specific sites in the inflammatory cascade are likely to have an impact in the near future. Among them, anti-inflammatory cytokines (recombinant IL-10 and IL-11), antibodies (humanized IgG4, anti-TNFalpha, anti-alpha4-integrin) and antisense therapies (ICAM-1) are currently being evaluated in CD treatment (Sandborn and Faubion 2004).

The frequency of indications for surgery parallels the frequency of local intestinal complications of the disease. Surgery is never curative for CD because the disease frequently recurs at or near the site of surgery; its overall goal is to conserve bowel and return the individual to the best possible quality of life. Up to 74% of all patients eventually require surgical intervention for their disease (Farmer 1985), and nearly 30% of patients require surgery within the first year of diagnosis (Podolsky 1991).

Although the etiology of CD is poorly understood, studies indicate that CD pathogenesis is the result of the complex interaction between environmental factors (i.e. gut micro-flora), genetic susceptibility, and the immune system. It has been proposed that IBD results from a dys-regulated mucosal immune response to the intestinal micro-flora in genetically susceptible individuals. The inappropriate activation of the mucosal immune system observed in CD has been linked to a loss of tolerance to gut commensals. It also appears that the loss of mucosal integrity leading to translocation of bacteria in the bowel wall is a crucial step for the propagation of the inflammatory process. However, it is not known whether barrier function is first compromised by intrinsic defects in epithelial integrity, by infection with enteric pathogens, or by loss of commensal-dependent signals necessary to maintain the physical integrity of the epithelium and hypo-responsiveness of the mucosal immune system (reviewed in Bouma and Strober 2003).

Familial aggregation, twin studies and consistent ethnic differences in disease frequency have strongly supported the important role of genetic factors in the cause of CD (reviewed in Andres and Friedman 1999). However, the incomplete concordance for CD within monozygotic twins, the phenotypic variations and the observed familial pattern of non-Mendelian inheritance suggest that CD has a complex genetic basis with many contributing genes. These facts also underline the presence and importance of environmental factors in the pathogenesis of this disease, such as gut micro-flora as mentioned above, and cigarette smoking which is the best known environmental factor for CD (reviewed in Andres and Friedman 1999). In addition, disease heterogeneity in the phenotype (location, age of onset, number and types of surgery, behavior, extra-intestinal manifestations, response to class of medications) can reflect extensive genetic heterogeneity.

Previously Identified Genes and Loci

Genetic studies have previously indicated the presence of several loci predisposing to Inflammatory Bowel Diseases. Nine IBD loci have been identified: IBD1 (CARD15/NOD2, Caspase recruitment domain family, member 15 (NOD2 protein)), IBD2 (Inflammatory bowel disease-2), IBD3 (Inflammatory bowel disease-3), IBD4 (Inflammatory bowel disease-4), IBD5 (Inflammatory bowel disease-5), IBD6 (Inflammatory bowel disease-6), IBD7 (Inflammatory bowel disease-7), IBD8 (Inflammatory bowel disease-8), and IBD9 (Inflammatory bowel disease 9).

Several loci have been identified and replicated to date; they are located on chromosomes 16q12, 12q13.2-q24.1, 6p21, 14q11-q12, 5q31-q33, 19p13, 1p36, 16p, and 3p26 respectively (Wild and Rioux 2004; Duerr et al., 2002). Results from linkage studies have suggested that CD and UC share some loci but do not share others, such as locus 16q12 which is unique to CD (The IBD International Genetics Consortium 2001). Most of the genes determining susceptibility in each of these chromosomal regions remain to be identified. The most widely replicated loci are on chromosomes 16 (caused by mutations in the NOD2/CARD15 gene), 12, 6, 14 and 5 (OCTN2 genes at IBD5 locus), which predispose to early-onset Crohn's disease. Thus, there is a continuing need in the medical arts for genetic markers of Crohn's disease and guidance for the use of such markers.

Several years ago, two different groups reported the association of CARD15 (NOD2) variants with CD (Hugot 2001; Ogura 2001). CARD15 is located at the IBD1 locus. The gene codes for an intracellular receptor involved in the innate immune detection of bacterial products. This detection induces the activation of the NF-kB pathway which is of particular importance in immune and inflammatory responses (Philpott and Viala 2004). Three major mutations represent 82% of the total CARD15 mutations: R702W, G908R, and 1007fsinsC. However they do not explain over 20% of the genetic predisposition to the disease, and altogether, they are carried by 30-50% of CD patients and 15-20% of healthy controls in Caucasian populations. These values are much lower in Japanese or Africans (reviewed in Girardin 2003).

Recently, the OCTN1 and OCTN2 genes at IBD5 locus have been associated with CD (Peltekova 2004). Variants in these genes are in strong linkage disequilibrium and create a two-allele risk haplotype enriched in patients with CD. Both proteins are trans-membrane sodium-dependent carnitine transporters and sodium-independent organic cation transporters. The variants may cause disease by impairing OCTN activity or expression, reducing carnitine transport in a cell-type and disease-specific manner (Peltekova 2004). The IBD5 locus contains multiple candidate genes including the genes for organic cation transporters (OCTN2/SLC22A4 and SLC22A5), the gene for a LIN4-domain-containing protein (RILIPDLIM3), the gene for the oc2 subunit of proline hydroxylase (P4HA2) and a gene of unknown function (NCBI UniGene identifier Hs.70932). Because of extensive linkage disequilibrium (LD) in this region, it has not been possible to further refine the SNP map and unambiguously identify a single susceptibility gene. With respect to OCTN2, the basal transcription is downregulated by the C allele of G-207C, and this allele disrupts the ability of SLC22A5 to be upregulated in response to heat shock or arachidonic acid as a result of impaired HSF I binding and subsequent transcriptional activation.

The mechanism by which these organic cation transporters contribute to the pathology of Crohn's disease may relate to the in vivo metabolic importance of carnitine. Carnitine facilitates transport of long chain fatty acids across the mitochondrial inner membrane for subsequent P-oxidation, and is also important in the maintenance of cellular CoenzymeA levels. Carnitine uptake into lymphocytes, along with a corresponding decrease in plasma levels, is a physiological response to inflammation. Symptoms of the related condition Ulcerative Colitis may be due to an energy deficiency in colonic epithelium secondary to poor mitochondrial function due to decreased long-chain fatty acid transport to the mitochondria (Roediger W E 1980). The haplotype of mutations in Crohn's disease patients provided previously might affect cellular metabolic energy levels in inflamed tissue by combining impaired OCTN1 transporter function with downregulation and inability to respond to heat or inflammatory stress by the OCTN2 gene. Heat shock proteins and arachidonic acids are involved in response to a variety of cellular stresses, including sepsis, metabolic stress and ischaemia. The heat shock response modulates inflammation through modulation of NF-kB activation. OCTN2 is a heat stress inducible protein. Thus, the OCTN2 gene is normally upregulated in response to inflammation through binding of HSF I protein to its promoter. This in turn mobilizes carnitine and bolsters metabolism in the inflamed tissue. Impaired OCTN1 carnitine transporter activity and lowered OCTN2 expression level results in reduced metabolism and either triggers or worsens cellular stress in areas of inflammation. The two OCTN transporters may therefore function in the inflammatory pathology of Crohn's disease. Additionally, OCTN1 is a polyspecific cation transporter, and might have a role in the uptake of drugs used to treat CD from the gut. As described above, OCTN2 is a transporter protein with the ability to transport carnitine in a sodium dependent manner. Missense mutations and nonsense mutations in the organic cation transporter OCTN2 had previously been identified in patients with primary Systemic Carnitine Deficiency (SCD), an autosomal recessive disorder characterized by progressive cardiomyopathy, skeletal myopathy, hypoglycemia and hyperammonemia.

Genes encoding proteins involved in the immune system, epithelial functions, and host response to micro-organisms represent good potential candidates for CD and have been examined in numerous case-control studies. However, many of the published associations of genetic variants with CD have not been replicated in follow-up studies.

The genetic variants that have been identified so far in CD explain only a fraction of the genetic predisposition to this disorder. It is clear that multiple components contribute to disease risk, each component having a modest effect on disease susceptibility. Thus the development of GeneMaps for CD may lead to a better understanding of pathogenesis and to the identification of new pathways involved in the disease, ultimately leading to better treatments for the patients. GeneMaps may also lead to molecular diagnostic tools that will identify subjects at risk for CD or for serious complications of the disease.

Genome Wide Association Study to Construct a GeneMap for Crohn's Disease

The present invention is based on the discovery of genes associated with Crohn's disease. In the preferred embodiment, disease-associated loci (candidate regions; Table 1) are identified by the statistically significant differences in allele or haplotype frequencies between the cases and the controls. For the purpose of the present invention, 31 candidate regions (Table 1) are identified, including two previously known regions. CARD15 (NOD2) (Hugot et al. 2001 Nature 411:599 and Ogura et al. 2001 Nature 411:603.) and OCTN (Peltekova et al. 2004 Nature Genetics 36:471) have been previously reported to be associated with Crohn's disease.

The invention provides a method for the discovery of genes associated with Crohn's disease and the construction of a GeneMap for Crohn's disease in a human population, comprising the following steps (see Example section herein):

Step 1: Recruit Patients (Cases) and Controls

In the preferred embodiment, 500 patients diagnosed for Crohn's disease along with two family members are recruited from the Quebec Founder Population (QFP). The preferred trios recruited are parent-parent-child (PPC) trios. Trios can also be recruited as parent-child-child (PCC) trios. In another preferred embodiment, more or less than 500 trios are recruited. In another embodiment, independent case and control samples are recruited.

In another embodiment, the present invention is performed as a whole or partially with DNA samples from individuals of another founder population than the Quebec population or from the general population.

In another embodiment, the present invention is performed as a whole or partially with DNA samples from individuals of another population such as a German population.

Step 2: DNA Extraction and Quantitation

Any sample comprising cells or nucleic acids from patients or controls may be used. Preferred samples are those easily obtained from the patient or control. Such samples include, but are not limited to blood, peripheral lymphocytes, buccal swabs, epithelial cell swabs, nails, hair, bronchoalveolar lavage fluid, sputum, or other body fluid or tissue obtained from an individual.

In one embodiment, DNA is extracted from such samples in the quantity and quality necessary to perform the invention using conventional DNA extraction and quantitation techniques. The present invention is not linked to any DNA extraction or quantitation platform in particular.

Step 3: Genotype the Recruited Individuals

In one embodiment, assay-specific and/or locus-specific and/or allele-specific oligonucleotides for every SNP marker of the present invention (Tables 2, 3, 4, 5, 6, 7 and 10) are organized onto one or more arrays. The genotype at each SNP locus is revealed by hybridizing short PCR fragments comprising each SNP locus onto these arrays. The arrays permit a high-throughput genome wide association study using DNA samples from individuals of the Quebec founder population. Such assay-specific and/or locus-specific and/or allele-specific oligonucleotides necessary for scoring each SNP of the present invention are preferably organized onto a solid support. Such supports can be arrayed on wafers, glass slides, beads or any other type of solid support.

In another embodiment, the assay-specific and/or locus-specific and/or allele-specific oligonucleotides are not organized onto a solid support but are still used as a whole, in panels or one by one. The present invention is therefore not linked to any genotyping platform in particular.

In another embodiment, one or more portions of the SNP maps (publicly available maps, proprietary maps from Perlegen Sciences, Inc. (Mountain View, Calif., USA), and our own proprietary QLDM map) are used to screen the whole genome, a subset of chromosomes, a chromosome, a subset of genomic regions or a single genomic region.

The 1,500 individuals composing the 500 trios are preferably individually genotyped with at least 80,000 markers, generating at least a few million genotypes; more preferably, at least a hundred million.

Step 4: Exclude the Markers that did not Pass the Quality Control of the Assay.

Preferably, the quality controls consist of, but are not limited to, the following criteria: eliminate SNPs that had a high rate of Mendelian errors (cut-off at 1% Mendelian error rate), that deviate from the Hardy-Weinberg equilibrium, that are non-polymorphic in the Quebec founder population or have too many missing data (cut-off at 1% missing values or higher), or simply because they are non-polymorphic in the Quebec founder population (cut-off at 1%≦10% minor allele frequency (MAF)).

Step 5: Perform the Genetic Analysis on the Results Obtained Using Haplotype Information as well as Single-Marker Association.

In the preferred embodiment, genetic analysis is performed on all the genotypes from step 3.

In another embodiment, genetic analysis is performed on a total of 248,535 SNPs.

In one embodiment, the genetic analysis consists of, but is not limited to features corresponding to Phase information and haplotype structures. Phase information and haplotype structures are preferably deduced from trio genotypes using Phasefinder. Since chromosomal assignment (phase) cannot be estimated when all trio members are heterozygous, an Expectation-Maximization (EM) algorithm may be used to resolve chromosomal assignment ambiguities after Phasefinder.

In yet another embodiment, the PL-EM algorithm (Partition-Ligation EM; Niu et al., Am. J. Hum. Genet. 70:157 (2002)) can be used to estimate haplotypes from the “genotype” data as a measured estimate of the reference allele frequency of a SNP in 15-marker windows that advance in increments of one marker across the data set. The results from such algorithms are converted into 15-marker haplotype files. Subsequently, the individual 15-marker block files are assembled into one continuous block of haplotypes for the entire chromosome. These extended haplotypes can then be used for further analysis. Such haplotype assembly algorithms take the consensus estimate of the allele call at each marker over all separate estimations (most markers are estimated 15 different times as the 15 marker blocks pass over their position).

In the preferred embodiment, the haplotypes for both the controls and the patients are derived in this manner. The preferred control of a trio structure is the spouse if the patient is one of the parents or the non-transmitted chromosomes (chromosomes found in parents but not in affected child) if the patient is the child.

In another embodiment, the haplotype frequencies among patients are compared to those among the controls using LDSTATS, a program that assesses the association of haplotypes with the disease. Such program defines haplotypes using multi-marker windows that advance across the marker map in one-marker increments. Such windows can be 1, 3, 5, 7 or 9 markers wide, and all these window sizes are tested concurrently. Larger multi-marker haplotype windows can also be used. At each position the frequency of haplotypes in cases is compared to the frequency of haplotypes in controls. Such allele frequency differences for single marker windows can be tested using Pearson's Chi-square with any degree of freedom. Multi-allelic haplotype association can be tested using Smith's normalization of the square root of Pearson's Chi-square. Such significance of association can be reported in two ways:

The significance of association within any one haplotype window is plotted against the marker that is central to that window.

P-values of association for each specific marker are calculated as a pooled P-value across all haplotype windows in which they occur. The pooled P-value is calculated using an expected value and variance calculated using a permutation test that considers covariance between individual windows. Such pooled P-values can yield narrower regions of gene location than the window data (see example 3 for details on analysis methods, such as LDSTATS v2.0 and v4.0).

In another embodiment, conditional haplotype analyses can be performed on subsets of the original set of cases and controls using the program LDSTATS. The selection of a subset of cases and their matched controls can be based on the carrier status of cases at a gene or locus of interest (see conditional analysis section in example 3 herein). Various conditional haplotypes can be derived, such as protective haplotypes and risk haplotypes.

Step 6: Fine Mapping

In this step, the candidate regions that were identified by step 4 are further mapped for the purpose of refinement and validation.

In the preferred embodiment, this fine mapping is performed with a density of genetic markers higher than in the genome wide scan (step 3) using any genotyping platform available in the art. Such fine mapping can be, but is not limited to, typing the allele via an allele-specific elongation assay that is then ligated to a locus-specific oligonucleotide. Such assays can be performed directly on the genomic DNA at a highly multiplex level and the products can be amplified using universal oligonucleotides. For each candidate region, the density of genetic markers can be, but is not limited to, a set of SNP markers with an average inter-marker distance of 1-4 Kb distributed over about 400 Kb to 1 Mb, roughly centered at the highest point of the GWS association. The preferred samples are those obtained from Crohn's disease PPC trios including the ones used for the GWS. Other preferred samples are trios or case control samples from another population, such as a German population.

In the preferred embodiment, the genetic analysis of the results obtained using haplotype information as well as single-marker association (as performed as in step 5, described herein) is performed as described herein (step 5 and Example section). The candidate regions that are validated and confirmed after this analysis proceed to a gene mining step described in Example 5, herein, to characterize their marker and genetic content.

Step 7: SNP and DNA Polymorphism Discovery

In the preferred embodiment, all the candidate genes and regions identified in step 6 are sequenced for polymorphism identification.

In another embodiment, the entire region, including all introns, is sequenced to identify all polymorphisms.

In yet another embodiment, the candidate genes are prioritized for sequencing, and only functional gene elements (promoters, conserved noncoding sequences, exons and splice sites) are sequenced.

In yet another embodiment, previously identified polymorphisms in the candidate regions can also be used. For example, SNPs from dbSNP, Perlegen Sciences, Inc., or others can also be used rather than resequencing the candidate regions to identify polymorphisms.

The discovery of SNPs and DNA polymorphisms generally comprises a step consisting of determining the major haplotypes in the region to be sequenced. The preferred samples are selected according to which haplotypes contribute to the association signal observed in the region to be sequenced. The purpose is to select a set of samples that covers all the major haplotypes in the given region. Each major haplotype is preferably analyzed in at least a few individuals.

Any analytical procedure may be used to detect the presence or absence of variant nucleotides at one or more polymorphic positions of the invention. In general, the detection of allelic variation requires a mutation discrimination technique, optionally an amplification reaction and optionally a signal generation system. Any means of mutation detection or discrimination may be used. For instance, DNA sequencing, scanning methods, hybridization, extension based methods, incorporation based methods, restriction enzyme-based methods and ligation-based methods may be used in the methods of the invention.

Sequencing methods include, but are not limited to, direct sequencing, and sequencing by hybridization. Scanning methods include, but are not limited to, protein truncation test (PTT), single-strand conformation polymorphism analysis (SSCP), denaturing gradient gel electrophoresis (DGGE), temperature gradient gel electrophoresis (TGGE), cleavage, heteroduplex analysis, chemical mismatch cleavage (CMC), and enzymatic mismatch cleavage. Hybridization-based methods of detection include, but are not limited to, solid phase hybridization such as dot blots, multiple allele specific diagnostic assay (MASDA), reverse dot blots, and oligonucleotide arrays (DNA Chips). Solution phase hybridization amplification methods may also be used, such as Taqman. Extension based methods include, but are not limited to, amplification refraction mutation systems (ARMS), amplification refractory mutation systems (ALEX), and competitive oligonucleotide priming systems (COPS). Incorporation based methods include, but are not limited to, mini-sequencing and arrayed primer extension (APEX). Restriction enzyme-based detection systems include, but are not limited to, restriction site generating PCR. Lastly, ligation based detection methods include, but are not limited to, oligonucleotide ligation assays (OLA). Signal generation or detection systems that may be used in the methods of the invention include, but are not limited to, fluorescence methods such as fluorescence resonance energy transfer (FRET), fluorescence quenching, fluorescence polarization as well as other chemiluminescence, electrochemiluminescence, Raman, radioactivity, colometric methods, hybridization protection assays and mass spectrometry methods. Further amplification methods include, but are not limited to self sustained replication (SSR), nucleic acid sequence based amplification (NASBA), ligase chain reaction (LCR), strand displacement amplification (SDA) and branched DNA (B-DNA).

Step 8: Ultrafine Mapping

This step further maps the candidate regions and genes confirmed in the previous step to identify and validate the responsible polymorphisms associated with Crohn's disease in the human population.

In a preferred embodiment, the discovered SNPs and polymorphisms of step 7 are ultrafine mapped at a higher density of markers than the fine mapping described herein using the same technology described in step 6.

Step 9: GeneMap Construction

The confirmed variations in DNA (including both genic and non-genic regions) are used to build a GeneMap for Crohn's disease. The gene content of this GeneMap is described in more detail below. Such GeneMap can be used for other methods of the invention comprising the diagnostic methods described herein, the susceptibility to Crohn's disease, the response to a particular drug, the efficacy of a particular drug, the screening methods described herein and the treatment methods described herein.

As is evident to one of ordinary skill in the art, all of the above steps or the steps do not need to be performed, or performed in a given order to practice or use the SNPs, genomic regions, genes, proteins, etc. in the methods of the invention.

Genes from the GeneMap

In one embodiment the GeneMap consists of genes and targets, in a variety of combinations, identified from the candidate regions listed in Table 1. In another embodiment, all genes from Tables 8, 9, 19, 20, 21, 22, 23 or 24 are present in the GeneMap. In another preferred embodiment, the GeneMap consists of a selection of genes from Tables 8, 9, 19, 20, 21, 22, 23 or 24. The genes of the invention (Tables 8, 9, 19, 20, 21, 22, 23 or 24) are arranged by candidate regions and by their chromosomal location. Such order is for the purpose of clarity and does not reflect any other criteria of selection in the association of the genes with Crohn's disease.

In another embodiment, the GeneMap consists of the non-limiting examples displayed in networks from FIGS. 1 to 24. Genes represented in these networks were selected as described below:

In the preferred embodiment, genes identified in the WGAS and subsequent fine mapping studies for Crohn's disease (CD) are evaluated using the Ingenuity Pathway Analysis application (IPA, Ingenuity systems) in order to identify direct biological interactions between these genes, and also to identify molecular regulators acting on those genes (indirect interactions) that could be also involved in CD. The purpose of this effort is to decipher the molecules involved in contributing to CD. These gene interaction networks are very valuable tools in the sense that they facilitate extension of the map of gene products that could represent potential drug targets for CD.

From the genetic analyses, 31 candidate regions were considered for the development of potential protein interaction networks involved in CD. These regions and their coordinates are presented in Table 1 of this patent application. Out of 31 regions, 4 regions were not included in this analysis because they did not contain any annotated genes. Tables 8 and 19 list the annotated genes present in the remaining 27 regions, and that were used for IPA analysis.

A total of 295 annotated genes were identified in the 27 fine-mapped regions (Tables 8 and 19), and were imported in the IPA software. In the preferred embodiment, the analysis can be performed by looking for direct interactions only. From this analysis 285 genes were mapped to the Ingenuity database and assigned to 23 networks as defined by IPA. These networks are based on functional relationships between gene products using known interactions in the literature. For each network, some nodes were manually extended to include good candidate genes that could play a role in the biochemical pathways of CD. Table 20 contains information about the gene content of each network, as well as the top functions assigned to those biochemical pathways.

In another embodiment, the analysis can be performed by looking for direct and indirect interactions. From this analysis 270 genes were mapped to the Ingenuity database and assigned to 17 genetic networks as defined by IPA. Table 21 contains information about the gene content of each network, as well as the top functions assigned to those biochemical pathways.

In yet another embodiment, a subset of the genes (61) mapping to the candidate regions can be used as input to the Ingenuity Pathway Analysis System (Table 22). These genes are selected according to criteria that included their relevance to the pathophysiology of the disease and location with respect to the statistical evidence. Tables 22 and 23 contain information about the gene content of each network, as well as the top functions assigned to those biochemical pathways.

### Results from Analyses Incorporating Direct Interactions for 295 Annotated Genes

Network 1 Direct Only

Network 1 contains 51 nodes (35 original and 16 manual additions) with 18 genes from the fine mapped regions (FIG. 1). A short description of these 18 genes follows. By virtue of their role in immune response and/or inflammation (CD5 and CD6, GNAI2), barrier integrity, protection, and function (CLTC, GPX1), gastrointestinal physiology (OPRK1), biochemical pathways involved in the disease pathogenesis (DCP1A, GRK5, KSR1, RGS20), some of these genes are very good candidates for a role in the pathophysiology of CD. GNAI2, GPX1, and KSR1 have even been linked to the onset of colitis in mouse models (see below).

The expression of 8 genes from this network has been shown to vary in some studies of gene expression profiling for CD. For example, in sigmoid colon tissue from Crohn's patients compared to control tissue, ADORA3 was downregulated, and CLTC was upregulated (Costello et al 2005). In the study by Langmann et al 2004, GRK5 and GNAI3 were shown to be upregulated in colons of Crohn's patients compared to control specimens; KCTD3 was upregulated in ileum, and CTBP2, GNB1, ENTH were downregulated in ileum.

BSN: This gene encodes Bassoon, a novel zinc-finger CAG/glutamine-repeat protein which localizes at the active zone of presynaptic nerve terminals. Both the presynaptic terminal and the postsynaptic compartment of neuronal synapses comprise a highly specialized cytoskeleton underlying the synaptic membranes. The presynaptic nerve terminal is the principal site of regulated neurotransmitter release. The active zone is the region of the presynaptic plasmalemma where synaptic vesicles dock, fuse, and release neurotransmitters. Tom Dieck et al. (1998) suggested that Bassoon may be involved in cytomatrix organization at the site of neurotransmitter release.

CD5 and CD6: lymphocyte antigen CD5, a human T-cell surface glycoprotein, is implicated in the proliferative response of activated T cells and in T-cell helper function. Its expression increases coordinately with that of cell surface CD3. Although expressed on lymphoid-committed progenitors, its expression is lost following natural killer (NK) cell differentiation. In contrast to its being a pan-T-cell marker, CD5 is only expressed on some B cells. It has been shown that CD5 up-regulation in B cells plays a role in tolerance to autoantigens. By setting the threshold level for activation signals, CD5 prevents B cells from activation-induced cell death and maintains tolerance in anergic B cells in vivo. The reason for keeping potentially autoreactive cells alive is that these cells are also necessary for an effective immune response to some pathogens. This supported the role of CD5 as a negative regulator of BCR signaling, which was later demonstrated by the generation of CD5-null mice. In these animals, peritoneal B cells, which are poorly responsive to BCR stimulation, restored their capacity to fully proliferate to anti-IgM (Gary-Gouy et al., 2002). Interestingly, Neil et al (1992) have reported a decrease in CD5+ B cells in peripheral blood of patients with CD. Finally, CD6 is a monomeric membrane glycoprotein that is involved in T cell activation.

CLTC: This gene encodes clathrin, heavy polypeptide (Hc). Clathrin is a major protein component of the cytoplasmic face of intracellular organelles, called coated vesicles and coated pits. These specialized organelles are involved in the intracellular trafficking of receptors and endocytosis of a variety of macromolecules. The basic subunit of the clathrin coat is composed of three heavy chains and three light chains. Recently, a potential role of endocytosis of junctional proteins in barrier disruption of intestinal epithelium has been reported (Ivanov et al 2004). This is important since barrier disruption in intestinal epithelium is one of the key features of CD. Finally, as previously mentioned, expression of CLTC is upregulated in sigmoid colon tissue from Crohn's patients compared to control tissue (Costello et al 2005).

DCP1A: DCP1 decapping enzyme homolog A. This gene encodes a decapping enzyme. Decapping is a key step in general and regulated mRNA decay. This protein and another decapping enzyme form a decapping complex, which interacts with the nonsense-mediated decay factor hUpf1 and may be recruited to mRNAs containing premature termination codons. This protein also participates in the TGF-beta signaling pathway (Bai et al 2002) which has been involved in CD (Monteleone et al 2001; Neurath et al 2002).

DDB1: damage-specific DNA binding protein 1, 127 kDa. This gene encodes the large subunit of DNA damage-binding protein which is a heterodimer composed of a large and a small subunit. This protein functions in nucleotide-excision repair. Its defective activity causes the repair defect in patients with xeroderma pigmentosum complementation group E (XPE).

GNAI2: guanine nucleotide binding protein (G protein), alpha inhibiting activity polypeptide 2. This protein plays a role in immune response; targeted deletion of this gene in mice induces lethal colitis closely resembling ulcerative colitis (Dalwadi et al 2004; Bjursten et at 2005; Wu et al 2005). In order to evaluate the possible involvement of this gene in IBD pathogenesis, Zhang et at (2000) looked at GNAI2 codon 179 sequences in 28 familial IBD patients, and 7 patients with colon cancer/dysplasia, from 12 multiplex IBD families. They could not find evidence of any mutation in the codon 179 of the GNAI2 gene.

GNAT1: guanine nucleotide binding protein (G protein), alpha transducing activity polypeptide 1. Transducin is a 3-subunit guanine nucleotide-binding protein which stimulates the coupling of rhodopsin and cGMP-phoshodiesterase during visual impulses. This gene encodes the alpha subunit in rods. Mutation of GNAT1 is a rare cause of specific congenital visual defect such as night blindness (Dryja et al 1996).

GPX1: The gene glutathione peroxidase 1 encodes a member of the glutathione peroxidase family. Glutathione peroxidase functions in the detoxification of hydrogen peroxide, and is one of the most important antioxidant enzymes in humans. GPX1 and GPX2 are the major enzymes that reduce hydroperoxides in intestinal epithelium. Esworthy et al (2001) have shown that mice with combined disruption of Gpx1 and Gpx2 genes have colitis, and their results suggest that GPX activity is essential for the prevention of the inflammatory response in intestinal mucosa.

GRK5: This gene encodes a member of the guanine nucleotide-binding protein (G protein)-coupled receptor kinase subfamily of the Ser/Thr protein kinase family. GRK5, or G protein-coupled receptor kinase 5, specifically phosphorylates the activated forms of G protein-coupled receptors. G protein-coupled receptor kinases (GRKs) play an important role in phosphorylating and regulating the activity of a variety of G protein-coupled receptors. Fan and Malik (2003) noted that desensitization of G protein-coupled receptors regulates the number of polymorphonuclear leukocytes (PMNs), as well as their motility and ability to stop upon contact with pathogens or target cells, and this desensitization is mediated by GRKs. They showed that the chemokine macrophage inflammatory protein-2 (MIP2) induces GRK2 and GRK5 expression in PMNs through PI3KG signaling. Also they showed that LPS-activated TLR4 signaling regulates PMN migration by modulating the expression of chemokine receptors in a GRK2- and GRK5-dependent manner. Also, members of the GRK family have been involved in the pathogenesis of inflammation (Johnson et al 2002; Lombardi et al 1999). In addition, since the autonomic nervous system can influence the immune response, it is interesting to note that GRK5 knockout in mouse results in cholinergic supersensitivity and impaired muscarinic receptor desensitization (Gainetdinov et al 1999). Thus it is likely that some kind of disregulation of GRK5 could impact the immune response. As previously mentioned, expression of GRK5 was shown to be upregulated in colons of Crohn's patients compared to control specimens (Langmann et al 2004).

KSR1: Kinase suppressor of Ras-1 (KSR1) (formerly KSR) is a recently identified member of the EGFR-Ras-Raf-1-MAPK signaling pathway. There is a general agreement that KSR1 functions to coordinate signaling of the Ras GTPase to its downstream effector, c-Raf-1. KSR1 interacts with several proteins that possess kinase activity, including c-Raf-1, MEK1, MAPK, C-TAK1 and with protein phosphatase 2A (PP2A). TNF plays a pathogenic role in inflammatory bowel diseases (IBDs), which are characterized by altered cytokine production and increased intestinal epithelial cell apoptosis. KSR1 protects intestinal epithelium from TNF-alpha-induced apoptosis, abrogating inflammatory bowel disease (IBD). KSR1 has an essential protective role in the intestinal epithelial cell during inflammation through activation of cell survival pathways (Yan et al 2004).

LSM8: LSM8 homolog, U6 small nuclear RNA associated. Sm-like proteins contain the Sm sequence motif, which consists of 2 regions separated by a linker of variable length that folds as a loop. The Sm-like proteins are thought to be important for pre-mRNA splicing.

OPRK1: opioid receptor, kappa 1. This gene is a member of the G protein-coupled receptor (GPCR) family. This kappa receptor, as well as the mu and delta members, has been shown to interact with the chemokine receptor CCR5 on the membrane of human or monkey lymphocytes (Suzuki et al 2002). This interaction could modulate receptor function. In addition, OPRK1 is localized to the enteric nervous system, and expression levels of the protein have been shown to be significantly increased during chronic intestinal inflammation in mice (Pol et al 2003). Recent evidence has also revealed the association of OPRK1 with the sodium-hydrogen exchanger regulatory factor SLC9A3R1 (also called NHERF). SLC9A3R1 is characterized by two tandem PDZ domains and a potential phosphorylation site. The protein binds the cytoskeleton proteins ezrin, radixin, moesin, and merlin. SLC9A3R1 has been implicated in diverse aspects of epithelial membrane biology and immune synapse formation in T cells. It has also been hypothesized that defective regulation of SLC9A3R1 could be involved in psoriasis (Helms et al 2003), another inflammatory disease which can share, to some extent, some common genetic control with CD.

RAD50: RAD50 homolog (S. cerevisiae). This gene encodes a protein highly similar to Saccharomyces cerevisiae Rad50, which is involved in DNA double-strand break repair. This protein, cooperating with its partners MRE11 and NBS1, is important for DNA double-strand break repair, cell cycle checkpoint activation, telomere maintenance, and meiotic recombination. Knockout studies of the mouse homolog suggest this gene is essential for cell growth and viability (Bender et al 2002).

RAPGEF6: RAPGEF6 is Rap guanine nucleotide exchange factor (GEF) 6. Activation of Ras-like GTPases is mediated by guanine nucleotide exchange factors (GEFs), which induce the dissociation of GDP to allow binding of the more abundant GTP.

RGS20: regulator of G-protein signaling 20. RGS proteins are regulatory and structural components of G protein-coupled receptor complexes. They are GTPase-activating proteins for Gi and Gq class G-alpha proteins. They accelerate transit through the cycle of GTP binding and hydrolysis and thereby accelerate signaling kinetics and termination. The regulation of expression of RGS proteins could be one mechanism by which TLR signaling could modify GPCR signaling in dendritic cells (DCs). It has been reported that engagement of TLR3 or TLR4 on monocyte-derived DCs induces RGS16 and RGS20, markedly increases RGS1 expression, and potently down-regulates RGS18 and RGS14 without modifying other RGS proteins (Shi et al 2004a). TLR signaling has been involved in CD pathogenesis (Cobrin and Abreu 2005), making RGS20 a good candidate to play a role in CD (Ouburg et al 2005).

SEPT8: Septin 8. SEPT8 is a member of the highly conserved septin family. Septins are GTPases that assemble as filamentous scaffolds. They are essential for active membrane movement such as cytokinesis and vesicle trafficking. SEPT8 may play a role in platelet granular secretion (Blaser et al 2004).

STARD3: START domain containing 3. This gene encodes a protein which participates in intracellular cholesterol trafficking (Zhang et al 2002).

Network 2 Direct Only

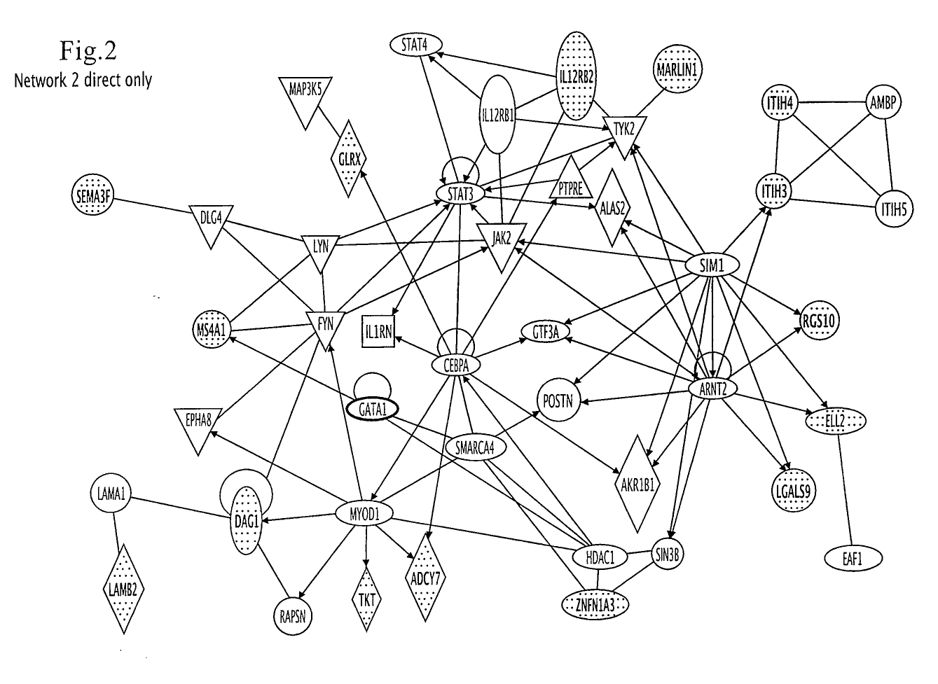

Network 2 contains 44 nodes (35 original and 9 manual additions) with 15 genes from the fine mapped regions (FIG. 2). A short description of these 15 genes follows. From their role in immune response and/or inflammation (IL12RB2, LGALS9, MS4A1, ZNFN1A3), barrier integrity, protection, and function (GLRX, LAMB2), some of these genes are very good candidates for a role in CD (see below). The expression of 7 genes from this network has been shown to vary in some studies of gene expression profiling. In the study by Langmann et al 2004, FYN, POSTN and SMARCA4 have been shown to be upregulated in colons of Crohn's patients compared to control specimens; TKT and LAMB2 were downregulated in colon; MS4A1 and LGALS9 were downregulated in both colon and ileum.

ADCY7: adenylate cyclase 7. The product of this gene is a member of the adenylyl cyclase class-4/guanylyl cyclase enzyme family that is characterized by the presence of twelve membrane-spanning domains in its sequences. ADCY7 is the major form of adenylyl cyclase in human platelets (Hellevuo et al 1993).

DAG1: The gene DAG1 (dystrophin-associated glycoprotein 1), encodes the 43-kD transmembrane and 156-kD extracellular dystrophin associated glycoprotein. Dystroglycan is a laminin binding component of the dystrophin-glycoprotein complex which provides a link between the subsarcolemmal cytoskeleton and the extracellular matrix. Dystroglycan has been suggested to play an important role in basement membrane assembly by binding soluble laminin and organizing it on the cell surface (Henry and Campbell, 1998). Striated muscle-specific disruption of the dystroglycan (DAG1) gene results in loss of the dystrophin-glycoprotein complex in differentiated muscle and a remarkably mild muscular dystrophy with hypertrophy and without tissue fibrosis (Crohn R D et al., 2002).

ELL2: elongation factor, RNA polymerase II, 2. This gene encodes a novel ELL-related RNA polymerase II elongation factor. ELL2 shows 49% identity and 66% similarity to ELL. Mechanistic studies indicated that ELL2 and ELL possess similar transcriptional activities. ELL is the second elongation factor to be implicated in oncogenesis, thus ELL2 could be involved in the control of cell growth.

GLRX: human glutaredoxin (thioltransferase), is known for its unique properties of specific and efficient catalysis of deglutathionylation of protein-S-S-glutathione-mixed disulfides (protein-SSG). These catalytic properties of glutaredoxin highlight its prominent role in homeostasis of protein sulfhydryl groups both in a protective mode under overt oxidative stress associated with aging and various disease states including cardiovascular and neurodegenerative diseases, diabetes, AIDS, cancer, as well as in a regulatory mode whereby reversible glutathionylation represents a mechanism of redox-activated signal transduction. These physiological roles are supported further by the documentation that glutaredoxin accounts for essentially all of the cellular protein-SSG deglutathionylase activity in mammalian cells and its inactivation by cadmium is correlated with inhibition of intracellular deglutathionylase activity. Glutaredoxins (Grx) are thiol-disulfide oxidoreductases with antioxidant capacity and catalytic functions closely associated with glutathione, an antioxidant abundantly present in human lung. Since the discovery that glucose deprivation-induced oxidative stress causes cytotoxicity in MCF-7/ADR cells, several studies have focused on determining the role of signal transduction pathways in the biological response. Several researchers had previously demonstrated that reactive oxygen species (ROS) could act as intracellular second messengers for inducing apoptosis. During glucose deprivation, supplementation of intracellular reduced thiol pools with N-acetyl-L-cysteine (NAC) was found to suppress metabolic oxidative stress-induced cellular responses including cytotoxicity, cytoskeletal reorganization, and stress-activated protein kinase (SAPK) activation. These results coupled with results using dominant negative c-Jun N-terminal kinase (JNK1) suggested that glucose deprivation-induced SAPK activation was in part responsible for cytotoxicity. Recent studies have also shown that pro-apoptotic signaling originating from the mitochondria is mediated by the JNK pathway possibly through Bid cleavage, cytochrome c release, and/or mitochondrial membrane depolarization. Redox-sensing molecules such as thioredoxin (TRX) and glutaredoxin (GRX) bind to apoptosis signal-regulating kinase 1 (ASK1) and suppress its activation. Glucose deprivation disrupts the interaction between TRX/GRX and ASK1 and subsequently activates the ASK1-stress-activated protein kinase/extracellular-signal-regulated kinase kinase-c-Jun N-terminal kinase 1 (JNK1) signal-transduction pathway. Thus, by its protective role against oxidative stress, GLRX may play an important role in protecting the integrity of intestinal epithelium.

IL12RB2: is interleukin 12 receptor beta 2 and is involved in auto immune and chronic inflammatory diseases (see US 20040009479A1 for details). The protein encoded by this gene is a type I transmembrane protein identified as a subunit of the interleukin 12 receptor complex. The biological functions of human IL-12 are mediated by the heterodimeric IL-12R composed of two subunits, the β1 and the β2 chains. Interleukin-12 (IL-12) is a proinflammatory cytokine that induces the production of interferon-gamma (IFN-g), favors the differentiation of T helper 1 (TH1) cells and forms a link between innate resistance and adaptive immunity. IL-12 has a direct role in the predisposition to human CD (Xwiers A et al, 2004). Both IL-12 and IL-23 are members of the IL-12 family of cytokines sharing a common p40 subunit. p40 forms heterodimers with p35 in IL-12 and p19 in IL-23. IL-12 and IL-23 bind to IL-12Rb1/IL-12Rb2 and IL-12Rb1/IL-23R, respectively. Engagement of IL-23 with its receptor activates a similar spectrum of Janus kinase (JAK)/signal transducer and activator of transcription (STAT) molecules as IL-12 but in contrast to IL-12, the most prominent STATs induced by IL-23 are STATS/STAT4 heterodimers rather than STAT4 homodimers (Trinchieri et al, 2003). The coexpression of this gene and IL12RB1 was shown to lead to the formation of high-affinity IL12 binding sites and reconstitution of IL12 dependent signaling. The expression of this gene is up-regulated by interferon gamma in Th1 cells, and plays a role in Th1 cell differentiation. The up-regulation of this gene is found to be associated with a number of infectious diseases, such as Crohn's disease and leprosy, which is thought to contribute to the inflammatory response and host defense.

ITIH3: inter-alpha (globulin) inhibitor H3. This gene encodes a member of the inter-alpha-trypsin inhibitors family of structurally related plasma serine protease inhibitors involved in extracellular matrix stabilization. Paris et al (2002) have shown inhibition of tumor growth and metastatic spreading by overexpression of inter-alpha-trypsin inhibitor family chains (ITI-L, -HI and -H3 chains). Thus they conclude that ITIH3 could have antitumoral or antimetastatic properties.

ITIH4: inter-alpha (globulin) inhibitor H4 (plasma Kallikrein-sensitive glycoprotein). A study reported that genetic variation at ITIH4 locus was likely one of the determinants of hypercholesterolemia (Fujita et al 2004).

LAMB2: laminin, beta 2 (laminin S). This gene encodes the beta chain isoform laminin, beta 2. The beta 2 chain contains the 7 structural domains typical of beta chains of laminin, including the short alpha region. Laminins form a family of extracellular matrix glycoproteins which, quantitatively, are the major noncollagenous constituent present in basement membranes. Laminins are composed of 3 non identical chains: laminin alpha, beta and gamma. They have been implicated in a wide variety of biological processes including cell adhesion, differentiation, migration and signaling . . . . Differential distribution of the various laminins have been observed during intestinal development and in the adult intestine, and important alterations in the pattern of laminin expression have been reported in various intestinal pathologies, such as tufting enteropathy, Crohn's disease and ulcerative colitis, and colorectal cancer (Teller and Beaulieu 2001). As mentioned previously, LAMB2 expression was downregulated in colon (Langmann et al 2004).

LGALS9: This gene encodes galectin 9 which is an S-type lectin. This galectin is strongly overexpressed in Hodgkin's disease tissue and may participate in the interaction between the H&RS cells with their surrounding cells and might thus play a role in the pathogenesis of this disease and/or its consistently associated immunodeficiency. The protein has N- and C-terminal carbohydrate-binding domains connected by a link peptide. Two isoforms (long and short) exist. The galectins are a family of beta-galactoside-binding proteins implicated in modulating cell-cell and cell-matrix interactions. Galectin-9 (Gal-9)/ecalectin was first cloned as a T cell-derived eosinophil chemoattractant (Matsumoto et al 1998). Also, galectin-9 is able to induce apoptosis of not only T cell lines but also of other types of cell lines, in a dose- and time-dependent manner, through the calcium-calpain-caspase-1 pathway. This suggests that galectin 9 may play a role in immunomodulation of T cell-mediated immune responses (Kashio et al 1998). As mentioned previously, LGALS9 was downregulated in both colon and ileum (Langmann et al 2004).

MARLIN1: now called JAKMIP1. MARLIN1 is a novel RNA-binding protein which associates with GABA receptors. GABA(B) receptors are heterodimeric G protein-coupled receptors that mediate slow synaptic inhibition in the central nervous system. Jamip1 (Jak and microtubule interacting protein), an alias of MARLIN1, was identified for its ability to bind to the FERM (band 4.1 ezrin/radixin/moesin) homology domain of Tyk2, a member of the Janus kinase (Jak) family of non-receptor tyrosine kinases that are central elements of cytokine signaling cascades. The restricted expression of Jamip1 and its ability to associate to and modify microtubule polymers suggest a specialized function of these proteins in dynamic processes, e.g. cell polarization, segregation of signaling complexes, and vesicle traffic, some of which may involve Jak tyrosine kinases (Steindler at al., 2004).

MS4A1: membrane-spanning 4-domains, subfamily A, member 1. This gene encodes a member of a gene family which is characterized by common structural features and similar intron/exon splice boundaries and display unique expression patterns among hematopoietic cells and nonlymphoid tissues. MS4A1 is a B-lymphocyte surface molecule which plays a role in the development and differentiation of B-cells into plasma cells. Also, it is functionally coupled to MHC Class II molecules (Leveille et al 1999).

RGS10: RGS10 encodes for the regulator of G-protein signaling 10. RGS family members are regulatory molecules that act as GTPase activating proteins for G alpha subunits of heterotrimeric G proteins. They drive G proteins into their inactive GDP-bound forms. RGS10 is a selective activator of G alpha i GTPase activity (Hunt et al 1996).

SEMA3F: sema domain, immunoglobulin domain (Ig), short basic domain, secreted, (semaphorin). SEMA3F is a secreted member of the semaphorin III family. This molecule could play a role in cell motility and adhesion, and can be a potent metastasis inhibitor (Bielenberg et al 2004).

TKT: transketolase. This gene encodes a thiamine-dependent enzyme that links the pentose phosphate pathway with the glycolytic pathway. Transketolase activity was found, among other tissues, in the epithelium of small intestine (Boren et al 2006). As mentioned previously, TKT expression was downregulated in colon (Langmann et al 2004).

ZNFN1A3: This gene (also known as Aiolos) encodes a member of the Ikaros family of zinc-finger proteins. Three members of this protein family (Ikaros, Aiolos and Helios) are hematopoetic-specific transcription factors involved in the regulation of lymphocyte development. This gene product is a transcription factor that is important in the regulation of B lymphocyte proliferation and differentiation. Both Ikaros and Aiolos can participate in chromatin remodeling. Regulation of gene expression in B lymphocytes by Aiolos is complex as it appears to require the sequential formation of Ikaros homodimers, Ikaros/Aiolos heterodimers, and Aiolos homodimers. Aiolos transcription factor controls cell death in T cells by regulating Bcl-2 expression and its cellular localization (Romero et al 1999). Also, the association of Aiolos and Bcl-xL has been shown to be involved in the control of apoptosis (Rebollo et al 2001). These findings suggest that ZNFN1A3 may play a role during the immune response events in CD.

Network 3 Direct Only

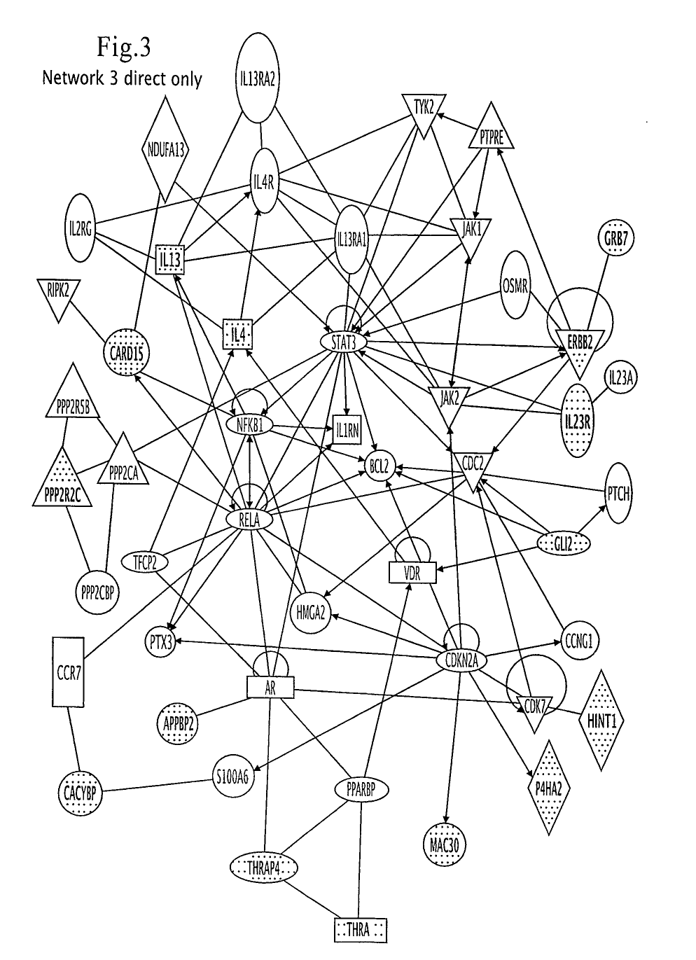

Network 3 contains 42 nodes (35 original and 7 manual additions) with 14 genes from the fine mapped regions (FIG. 3). A short description of these 14 genes follows. From their role in immune response and/or inflammation (CARD15, ERBB2, IL13, IL23R, PPP2R2C), gastrointestinal physiology (THRA), and biochemical pathways involved in the disease pathogenesis (GRB7, HINT1), some of these genes are very good candidates to play a role in CD (see below).

The expression of 9 genes of this network has been shown to vary in some studies of gene expression profiling for CD. In sigmoid colon tissue from Crohn's patients compared to control tissue, RELA was upregulated (Costello et al 2005). In the study by Langmann et al 2004, CCR7 has been shown to be downregulated in ilea of Crohn's patients compared to control specimens; GLI2, CCNG1, APPBP2 were upregulated in colon; PTCH was upregulated in both colon and ileum; IL2RG was downregulated in both colon and ileum. Contradictory results have been found for PPP2CA: it has been shown to be downregulated in colon in the study of Costello et al 2005, and upregulated in colon and ileum in the study of Langmann et al 2004.

APPBP2: This gene encodes the amyloid beta precursor protein (cytoplasmic tail) binding protein 2. The protein encoded by this gene interacts with microtubules and is functionally associated with beta-amyloid precursor protein transport and/or processing. The beta-amyloid precursor protein is a cell surface protein with signal-transducing properties, and it is thought to play a role in the pathogenesis of Alzheimer's disease. This gene has been found to be highly expressed in breast cancer. Multiple polyadenylation sites have been found for this gene. As already discussed above, APPBP2 expression was upregulated in colon (Langmann et al 2004).

CACYBP: The calcyclin binding protein encoded by this gene may be involved in calcium-dependent ubiquitination and subsequent proteosomal degradation of target proteins. It is a component of ubiquitin E3 complexes (Santelli E et al. 2005) that targets beta-catenin for destruction in response to p53 activation. This pathway links genotoxic injury to the destruction of beta-catenin and contributes to the reducing activity of Tcf/LEF transcription factors and to cell cycle arrest (Fukushima et al. 2006). Calcyclin binding protein may also play a role in gastric cancer drug resistance (Hu W et al. 2002; Shi Y et al. 2004b).

CARD15: CARD 15 (caspase recruitment domain family, member 15) is a member of a superfamily of genes, the nucleotide binding site-leucine-rich repeat (LRR) proteins, which are involved in intracellular recognition of microbes and their products. The protein is primarily expressed in peripheral blood leukocytes and plays a role in the immune response to intracellular bacterial lipopolysaccharides (LPS) by recognizing the muramyl dipeptide (MDP) derived from them and activating the NFKB protein. Since the first reports on the association of mutations in this gene with Crohn's disease (Hugot et al 2001; Ogura et al 2001), numerous laboratories have replicated these findings. CARD15/NOD-induced signal transduction is usually followed by nuclear factor (NF) kB activation; CARD15 induces NF-kappa-B via RICK (CARDIAK, RIP2) and IKK-gamma. Ogura et al. (2001) obtained cDNAs encoding NOD2 and showed that expression of NOD2 or NOD2B resulted in NFKB activation, and mutants lacking the LRRs had enhanced NFKB activation. The authors determined that both intact CARD domains are necessary and sufficient for IKK-gamma and RICK -dependent NFKB activation.

ERBB2: ERBB2 (NEU, HER2, V-erb-b2 erythroblastic leukemia viral oncogene homolog 2) forms a complex with the IL6 receptor (IL6R) in an IL6-dependent manner (Qiu et al., 1998). This association is important because the inhibition of ERBB2 activity resulted in abrogation of IL6-induced MAPK activation. Thus, ERBB2 is a critical component of IL6 signaling through the MAP kinase pathway. These findings showed how a cytokine receptor can diversify its signaling pathways by engaging with a growth factor receptor kinase. It is important to mention that in IBD, IL6 is produced by activated monocytes, macrophages and epithelial cells (Kusugami et al 1995). Fukushige et al. (1986) also observed amplification and elevated expression of ERBB2 in a gastric cancer cell line. Recently, in a gastric tumor, the Cancer Genome Project and Collaborative Group (2004) identified a 2326G-A transition in the ERBB2 gene that caused a gly776-to-ser (G776S) substitution.

GLI2: GLI-Kruppel family member GLI2. This gene encodes a protein which belongs to the C2H2-type zinc finger protein subclass of the Gli family. There are three known human GLI proteins: GLI-1, GLI-2, and GLI-3. The GLI genes are the main transcriptional mediators of the Hedgehog pathway in vertebrates. GLI1 and GLI2 are primarily transcriptional activators of Hedgehog target genes, while GLI3 is primarily a transcriptional repressor of Hedgehog targets. Shh (Sonic hedgehog) regulates gastric epithelial cell differentiation. Sonic hedgehog (SHH), Desert hedgehog (DHH) and Indian hedgehog (IHH) bind to Patched family receptors (PTCH1 and PTCH2) to transduce signals to GLI1, GLI2 and GLI3. GLI family transcription factors then activate transcription of Hedgehog target genes, such as FOXE1 and FOXM1 encoding. Forkhead-box transcription factors. The Hedgehog signaling pathway plays a pivotal role in a variety of human tumors, such as gastric cancer, pancreatic cancer, colorectal cancer, breast cancer, prostate cancer, basal cell carcinoma and brain tumors. Transgenic mice overexpressing the transcription factor GLI2 in cutaneous keratinocytes develop multiple skin tumors that are grossly indistinguishable from human basal cell carcinomas (BCCs). These tumors express the same protein markers as human BCCs and exhibit strikingly elevated levels of Ptch1 and Gli1, a hallmark of human BCCs but not squamous tumors. Gli2 is thus a potent oncogene in skin and suggests a pivotal role for this transcription factor in the development of BCC, the most common cancer in humans. GLI-2 also modulates retroviral gene expression. GLI-2/THP either activates or suppresses gene expression, depending on the promoter, but the same domain (first zinc finger) mediates both effects (Smith et al. 2001). One isoform of human GLI-2 appears to be identical to a factor previously called Tax helper protein (THP), thus named due to its ability to interact with a TG-rich element in the human T-Iymphotropic virus type 1 (HTLV-1) enhancer thought to mediate transcriptional stimulation by the Tax protein of HTLV-1. GLI-2/THP has been shown to interact with a DNA promoter element in HTLV-1 that is similar to the peri-ets (pets) site of the HIV-2 enhancer, the latter being an enhancer element that is induced following T-cell and monocytic activation. GLI-2/THP is a Tat cofactor which markedly activates HIV transcription (Browning C M et al., 2001). Loss-of-function mutations in the human GLI2 gene are associated with pituitary anomalies and holoprosencephaly-like features (Roessler et al. 2003). Also Kimmel et al (2000) showed that Gli2 and Gli3 play an important role in the normal development of murine hindgut. As mentioned previously, GLI2 expression was upregulated in colon (Langmann et al 2004).

GRB7: GRB7 is homologous to ras-GAP (ras-GTPase-activating protein). The product of this gene belongs to a small family of adapter proteins that are known to interact with a number of receptor tyrosine kinases and signaling molecules. This gene defines the GRB7 family, whose members include the mouse gene Grb10 and the human gene GRB14. Tanaka et al. (1998) found that the wild type GRB7 protein, but not the GRB7V isoform, was rapidly tyrosyl phosphorylated in response to EGF stimulation in esophageal carcinoma cells. Analysis of human esophageal tumor tissues and regional lymph nodes with metastases revealed that GRB7V was expressed in 40% of GRB7-positive esophageal carcinomas. GRB7V expression was enhanced after metastatic spread to lymph nodes as compared to the original tumor tissues. Transfection of an antisense GRB7 RNA expression construct lowered endogenous GRB7 protein levels and suppressed the invasive phenotype exhibited by esophageal carcinoma cells. These findings suggested that GRB7 isoforms are involved in cell invasion and metastatic progression of human esophageal carcinomas. Grb7 protein has been identified as a substrate of the epidermal growth factor receptor (EGFR) and related ERBB2 receptor-linked tyrosine kinase activity (Tanaka et al 1997). GRB7 interacts also with ephrin receptors. The protein plays a role in the integrin signaling pathway and cell migration by binding with focal adhesion kinase (FAK).

HINT1: This gene encodes a histidine triad nucleotide binding protein 1. It is a protein kinase C inhibitor and is a member of the HIT family of proteins. Because PKC signaling is involved in some inflammatory pathways, this protein could play a role in CD pathogenesis.

IL13: IL13 is a major stimulator of inflammation and tissue remodeling at sites of Th2 inflammation (Chen et al., 2005). In Th2 cells, production of the cytokines IL-4, IL-5, and IL-13 is controlled by cooperation between two families of transcription factors, the GATA and NFAT families (Monticelli et al., 2004). The calcium-regulated transcription factor NFAT consists of four family members, three of which (NFAT1 (p, c2), NFAT2 (c, c1), NFAT4 (x, c3)) are expressed in both cell types. Targeted disruption of the genes encoding individual NFAT family members suggests that there are cell type- and gene-specific differences in their ability to regulate gene transcription in activated cells. The GATA family of transcription factors is also essential for IL-4, IL-5, and IL-13 by Th2 cells, as shown by increased and decreased expression of these cytokines in transgenic mice overexpressing wild-type GATA3 and a dominant-negative version of GATA3 respectively.

IL23R: IL23R is a receptor for the heterodimeric cytokine interleukin 23. IL23 is a key cytokine controlling inflammation in peripheral tissue. The IL23R protein pairs with the receptor molecule IL12RB1/IL12Rbeta1, and both are required for IL23A signaling. The IL23 receptor (IL23R) comprises a novel receptor subunit (IL23R, that binds p19, and ILI2RP1, that binds p40 (Parham, et al., 2002). These two receptor subunits form the functional signaling complex and are expressed on CD4 and CD45Rb memory T cells as well as interferon gamma (IFNgamma) activated bone marrow macrophages (Parham, et al., supra). Recently, Mannon et al. (2004) reported that treatment with a monoclonal antibody against interleukin-12 may induce clinical responses and remissions in patients with Crohn's disease. The treatment resulted in decreases in Th1-mediated inflammatory cytokines at the site of the disease. Also, Becker et al. (2003) have shown that IL-23 is involved in the predisposition of the terminal ileum to develop chronic inflammatory responses and thus may provide a molecular explanation for the preferential clinical manifestation of Crohn's disease in this part of the gut.

MAC30: now called TMEM97. Meningioma-associated protein, MAC30, is a protein with unknown function and cellular localization that is differentially expressed in certain malignancies (Kayed et at 2004). Kayed et al showed that MAC30 mRNA levels were significantly increased in breast and colon cancer, but significantly decreased in pancreatic and renal cancer. Interestingly, TGF-beta down-regulated MAC30 mRNA levels in certain pancreatic cancer cells. MAC30 protein was localized in normal colon, gastric and esophageal tissues, especially in the mucosal cells.

P4HA2: procollagen-proline, 2-oxoglutarate 4-dioxygenase (proline 4-hydroxylase), alpha polypeptide II. This gene encodes a component of prolyl 4-hydroxylase, a key enzyme in collagen synthesis composed of two identical alpha subunits and two beta subunits. The encoded protein is one of several different types of alpha subunits and provides the major part of the catalytic site of the active enzyme. In collagen and related proteins, prolyl 4-hydroxylase catalyzes the formation of 4-hydroxyproline that is essential to the proper three-dimensional folding of newly synthesized procollagen chains.

PPP2R2C: The product of the gene is the regulatory subunit B of the protein phosphatase 2A (PP2A) which is one of the four major serine/threonine protein phosphatases regulating cell growth and division. The B-subunit is involved in enzyme activity and substrate specificity. The formation of IKK.PP2A complexes is required for the proper induction of IkappaB kinase (Kray et al. 2005) which inactivates NF-kappaB. Inappropriate activation of NFKB has been associated with a number of inflammatory diseases while persistent inhibition of NFKB leads to inappropriate immune cell development or delayed cell growth. PP2A plays another role in the resolution of inflammation by inducing neutrophil apoptosis (Alvarado-Kristensson et al. 2005).

THRA: The protein encoded by this gene is one of several thyroid hormone receptors. The triiodothyronine (T3) which links to THRA is a critical regulator of intestinal epithelial development and homeostasis by inducing activation of the enterocyte differentiation marker, intestinal alkaline phosphatase (Malo et al. 2004).

THRAP4: The thyroid hormone receptor-associated proteins (TRAPs) form a complex with the thyroid hormone receptor (TR). The human thyroid hormone receptor-associated protein (TRAP)-mediator is a coactivator for a broad range of nuclear hormone receptors as well as other classes of transcriptional activators.

Network 4 Direct Only

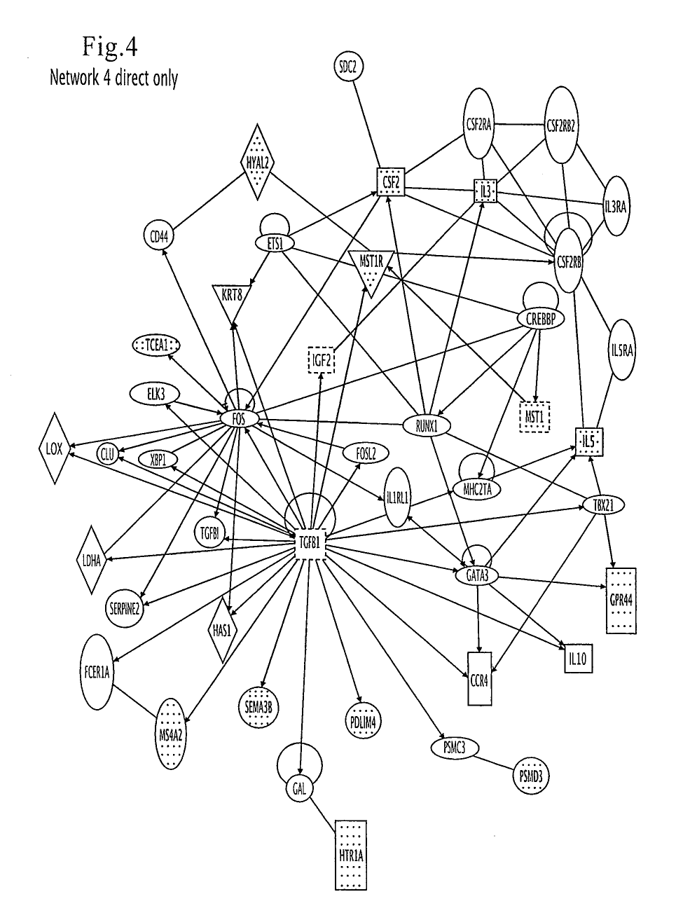

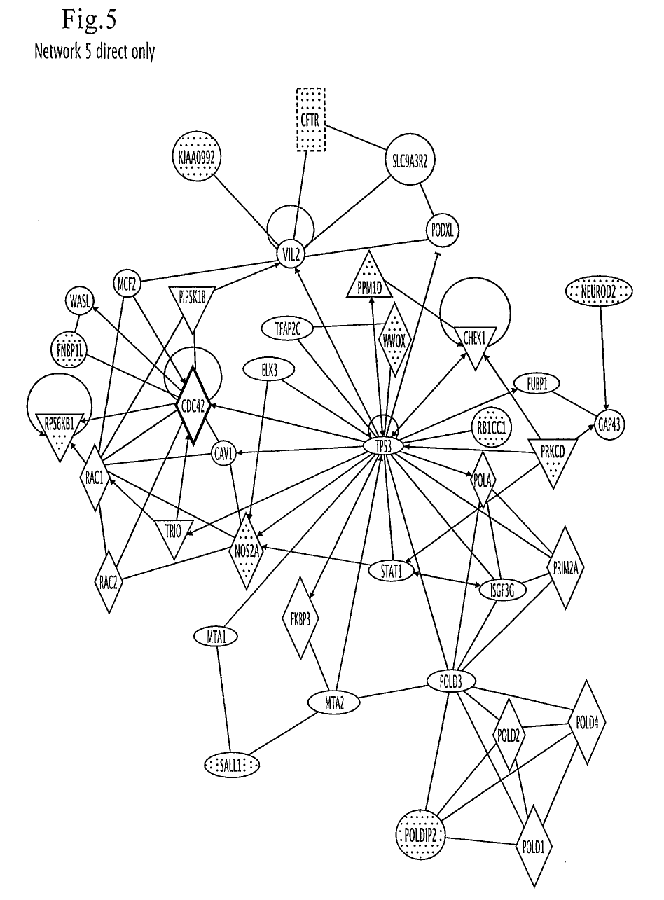

Network 4 contains 44 nodes (35 original and 9 manual additions) with 13 genes from the fine mapped regions (FIG. 4). A short description of these 13 genes follows. From their role in immune response and/or inflammation (CSF2, GPR44, IL3, IL5, MS4A2, MST1, MST1R), barrier integrity, protection, and function (HYAL2), gastrointestinal physiology (HTR1A), biochemical pathways involved in the disease pathogenesis (PDLIM4), some of these genes are very good candidates to play a role in CD (see below). The expression of 13 genes of this network has been shown to vary in some studies of gene expression profiling for CD. In sigmoid colon tissue from Crohn's patients compared to control tissue, XBP1 was upregulated (Costello et al 2005). In the study by Langmann et al 2004, CLU and MHC2TA have been shown to be downregulated in ilea of Crohn's patients compared to control specimens; KRT8, HYAL2, and MST1R were downregulated in colon; ELK3 was upregulated in both colon and ileum; MST1 and TGFBI were upregulated in ileum; FOS, ETS1 and CREBBP were upregulated in colon. Contradictory results have been found for PSMD3: it has been shown to be upregulated in colon in the study of Costello et al 2005, and downregulated in colon in the study of Langmann et al 2004.

CSF2: CSF2 belongs to the GM-CSF family and is a cytokine that stimulates the growth and differentiation of hematopoietic precursor cells from various lineages, including granulocytes, macrophages, eosinophils and erythrocytes. The active form of the protein is found extracellularly as a homodimer. Agnholt et al (2001) described an increase in GM-CSF production in CD, and highlighted this molecule as a possible target for infliximab treatment.

GPR44 (CRTH2): G Protein-coupled receptors (GPCRs), such as GPR44, are integral membrane proteins containing 7 putative transmembrane domains (TMs). These proteins mediate signals to the interior of the cell via activation of heterotrimeric G proteins that in turn activate various effector proteins, ultimately resulting in a physiologic response. CRTH2 is intriguing in that it is selectively expressed in Th2 cells, T cytotoxic type 2 cells, eosinophils, and basophils. Furthermore, CRTH2 can mediate intracellular Ca2+mobilization in response to a factor(s) released from activated mast cells, suggesting that CRTH2 may be closely involved in mast cell-mediated allergic inflammation (Hirai et al., 2002).

HTR1A: HTR1A is a serotonin receptor (5-hydroxytryptamine (serotonin) receptor 1A) which belongs to the family of GPCRs. Serotonin systems appear to play a key role in the pathogenesis of major depression and the therapeutic mechanisms of antidepressants. In the intestine, regulated release of serotonin from enterochromaffin cells activates neural reflexes that are involved in gut motility, secretion, vascular perfusion and sensation. A study reported that intestinal cellular structures responsible for the synthesis and storage of dopamine, norepinephrine, and serotonin may be affected by the inflammatory process in both CD and ulcerative colitis (Magro et al 2002). Also, it has been suggested that altered 5-HT signaling could be a contributing factor in altered gut function and sensitivity in inflammatory bowel disease (Linden et al 2005).

HYAL2: hyaluronoglucosaminidase 2. Hyaluronidase degrades hyaluronan, one of the major glycosaminoglycans of the extracellular matrix. Hyaluronan may play a role in cell proliferation, migration and differentiation. Elevated levels of hyaluronan are associated with numerous inflammatory diseases including inflammatory bowel disease. Recently, it as been shown that in human jejunum-derived mesenchymal cells, treatment with IL-1beta induced about a 30-fold increase in the levels of hyaluronan in the culture medium (Ducale et al 2005). As mentioned above, HYAL2 expression was downregulated in colon (Langmann et al 2004).

IL3: The IL3 gene encodes interleukin 3 which is a potent growth promoting cytokine. This cytokine is capable of supporting the proliferation of a broad range of hematopoietic cell types. It is involved in a variety of cell activities such as cell growth, differentiation and apoptosis. Ligumsky et al (1997) investigated the expression pattern of IL-3 in intestinal mast cells derived from steroid-treated IBD patients. Their results supported the idea that the down-regulation of IL-3 in mast cells derived from steroid-treated IBD patients occurs in vivo and could be an important mechanism for immunomodulation in IBD. Also, IL3 and GM-CSF have been shown to potentiate interferon-gamma-mediated endothelin production by human monocytes (Salh et al 1998).

IL5: IL5, interleukin 5, is also known as B cell differentiation factor I; T-cell replacing factor; and eosinophil differentiation factor. Intestinal parasitic diseases are commonly accompanied with diarrhoeal symptoms and allergic reactions. Eosinophilia occurs as a result of IL-5 synthesized from Th2 cells during allergic reactions. Eosinophilia is caused by the effect of IL-5 synthesized from Th2 cells. IL-5 is the most important cytokine in the transformation and development of eosinophils, and acts as an “eosinophil activator”. One of the significant causes of the increase in the amount of eosinophils in blood is parasitic diseases. Toxiallergic effects of certain parasites on the host's organism lead to an increase especially in eosinophil numbers. It is suggested therefore that the function of IL-5 and eosinophils is to protect against repeated exposure to gastrointestinal parasites (Ustun et al., 2004). On the other hand the eosinophilia observed may represent an immunopathological rather than a protective response and may merely be a consequence of the generalized inflammation induced by the Th2 response following infection with parasites. Approximately 25% of patients with IBD had a history of infectious enteritis. Microbial agents including parasites could increase the number of mast cells within the colonic muscle wall, release pro-inflammatory substances, and increase the number of inflammatory cells that might cause IBD.

MS4A2: MS4A2 gene encodes for the membrane-spanning 4-domains, subfamily A, member 2 (Fc fragment of IgE, high affinity I, receptor for; beta polypeptide. The high affinity IgE receptor is responsible for initiating the allergic response. Binding of allergen to receptor-bound IgE leads to cell activation and the release of mediators (such as histamine) responsible for the manifestations of allergy. The receptor is a tetrameric complex composed of an alpha chain, a beta chain, and 2 disulfide-linked gamma chains. It is found on the surface of mast cells and basophils. The alpha and beta subunits have not been detected in other hematopoietic cells, although the gamma chains are found in macrophages, natural killer (NK) cells, and T cells where they associate with the low affinity receptor for IgG or with the T-cell antigen receptor. Mast cells (MCs) express the high-affinity immunoglobin E (IgE) receptor (FcRI) on their surface, and they can be activated to secrete a variety of biologically active mediators by cross-linking of receptor-bound IgE. The release of mediators from MCs is responsible for the IgE-dependent allergic reactions clinically recognized as anaphylactic reactions, acute asthma, and allergic rhinitis. Sandford et al. (1993) demonstrated that the gene is located on 11q13. Furthermore, they demonstrated that the FCER1B gene is linked to clinical atopy. In their linkage study of atopy, Sandford et al. (1993) used only maternally derived alleles; paternally derived alleles failed to show linkage. The known roles of the high-affinity IgE receptor in antigen-induced mast-cell degranulation and in the release of cytokines that enhance IgE production, taken with the location in the same region, 11q13, made the FCER1B gene a candidate for the chromosome 11 atopy locus. Most MS4A genes encode proteins with at least 4 potential transmembrane domains and N- and C-terminal cytoplasmic domains encoded by distinct exons. The exception is MS4A6E, which encodes a protein with only 2 transmembrane domains and no C-terminal cytoplasmic domain. Northern blot analysis revealed weak expression of MS4A4 in mouse colon and intestine but detected no expression in human tissues (Ishibashi et al. (2001).

MST1: Macrophage stimulating 1 (hepatocyte growth factor-like, HGFL, MSP), is an inflammatory cytokine able to activate macrophages and to interact with other inflammatory cytokines. MST1 has been proposed to participate in a wide spectrum of biological processes such as inflammation, tissue remodeling/wound healing, hematopoiesis, and bone formation. Both HGFL1/1 and HGFL2/2 mice demonstrate similar clinical (e.g. diarrhea, weight loss) and histologic involvement of the gastrointestinal tract after an administration of dextran sulfate sodium, suggesting that MST1 may aid in the local response to injury. Based on mRNA expression of stk in the colon, and on the ability of MST1 to stimulate proliferation of colonic epithelial cells in culture, an important role of MST1 in the gastrointestinal mucosa response to infectious or other inflammatory processes (e.g. chronic toxic injury) is suggested. As mentioned above, MST1 expression was upregulated in ileum (Langmann et al 2004).

MST1R: Macrophage stimulating 1 receptor (c-met-related tyrosine kinase, RON) gene, encodes a protein tyrosine kinase receptor comprised of an extra-cellular domain that contains the ligand binding pocket and an intracellular region where the kinase domain is located. It controls cell survival and motility programs related to invasive growth (Angeloni D. et al., 2003). A recent report indicates that the epithelial-cell transforming activity of JSRV Env depends on activation of the cell-surface receptor tyrosine kinase Mst1r (called RON for the human and Stk for the rodent orthologs). MST1 is a ligand of the Met-related MST1-Receptor (MST1R, RON). Although MST1-deficient mice are viable, MST1R is essential in mice before gastrulation for implantation, and is a known oncogene in man. The human receptor tyrosine kinases MET and MST1R (and their respective orthologs in other species) form a unique, two-member gene family that encodes proteins with identical modular structure that may perform similar functions. The expression of MST1R in human tissues and cell lines was examined and MST1R was found to be expressed in colon, skin, lung and bone marrow, and in granulocytes and adherent monocytes (WO1997US0005216). As mentioned above, MST1R expression was downregulated in colon (Langmann et al 2004).

PDLIM4: This gene encodes the LIM domain protein RIL. This small adaptor protein consists of two segments, the C-terminal LIM and the N-terminal PDZ domain, which mediate multiple protein-protein interactions. The RIL LIM domain can interact with PDZ domains in the protein tyrosine phosphatase PTP-BL and with the PDZ domain of RIL itself. It has also been shown to interact with TRIP6 (Cuppen 2000). TRIP6 is also a RIPK2-associated common signaling component of multiple NF-kappaB activation pathways as shown by Li et al (2005). As highlighted in network 3, RIPK2 interacts with CARD15 (Abbott et al 2004). Thus this gene could be involved in the signaling pathways of CD pathogenesis.

PSMD3: proteasome (prosome, macropain) 26S subunit, non-ATPase, 3. Proteasomes are distributed throughout eukaryotic cells at a high concentration and cleave peptides in an ATP/ubiquitin-dependent process in a non-lysosomal pathway.

SEMA3B: sema domain, immunoglobulin domain (Ig), short basic domain, secreted, (semaphorin) 3B. This family member is important in axonal guidance and has been shown to act as a tumor suppressor by inducing apoptosis and inhibiting cellular proliferation. Semaphorins have important roles in a variety of tissues. A common theme in the mechanisms of semaphorin function is that they alter the cytoskeleton and the organization of actin filaments and the microtubule network.

TCEA1: transcription elongation factor A (SII), 1. Transcription elongation factors help RNA polymerase II to transcribe past blockages due to specific DNA sequences, DNA-binding proteins, and transcription-arresting drugs.

Network 5 Direct Only

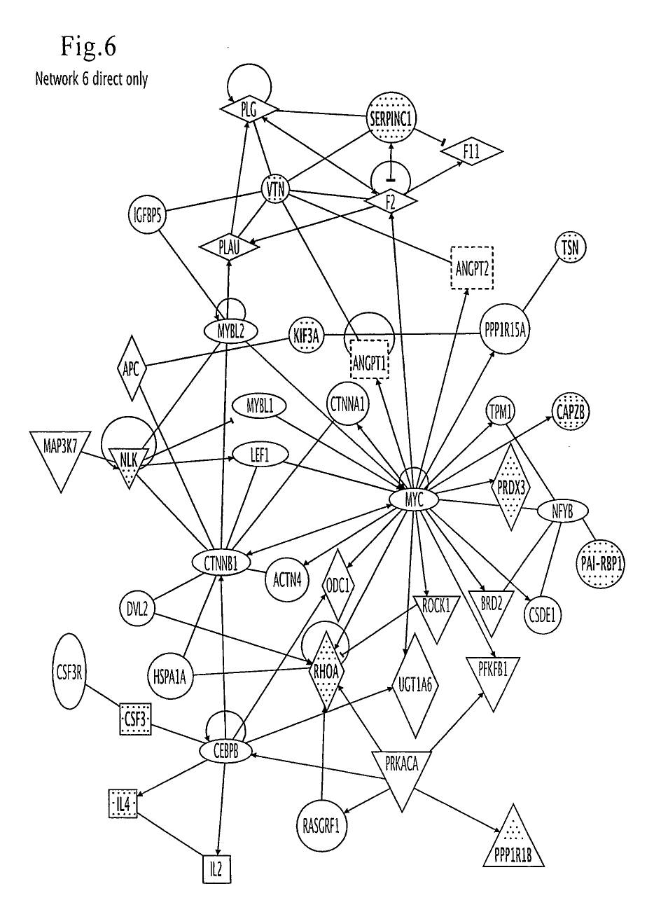

The network 5 contains 40 nodes (35 original and 5 manual additions) with 12 genes from the fine mapped regions (FIG. 5). A short description of these 12 genes follows. From their role in immune response and/or inflammation (NOS2A, PRKCD), barrier integrity, protection, and function (CFTR, KIAA0992, SALL1), some of these genes are very good candidates to play a role in CD (see below).

The expression of 17 genes of this network has been shown to vary in some studies of gene expression profiling for CD. In sigmoid colon tissue from Crohn's patients compared to control tissue, NOS2A was upregulated and FUBP1 downregulated (Costello et al 2005). In the study by Langmann et al 2004, RAC2 has been shown to be downregulated in ilea of Crohn's patients compared to control specimens; PRKCD and POLD4 were downregulated in colon; WASL, ELKS, WWOX, and FNBP1L, were upregulated in both colon and ileum; CAV1, PRIM2A and POLD2 were up regulated in ileum; CFTR, PODXL, STAT1, TRIO, and CDC42 were upregulated in colon.

CFTR: cystic fibrosis transmembrane conductance regulator, ATP-binding cassette (sub-family C (MRP), member 7). This gene encodes a member of the ATP-binding cassette (ABC) transporter family, which transports various molecules across cellular membranes. As a member of the MRP subfamily, the CFTR protein functions as a chloride channel localized primarily at the apical or luminal surfaces of epithelial cells that line the airway, gut and exocrine glands, controls the regulation of other transport pathways and is involved in multi-drug resistance. Mutations in this gene are associated with the autosomal recessive disorders cystic fibrosis and congenital bilateral aplasia of the vas deferens and result in both impaired Cl(−) secretion and enhanced Na(+) absorption in the colon of cystic fibrosis (CF) patients. Intestinal inflammation modulates the expression of CFTR and may contribute to diarrhea in ulcerative colitis both by increasing transepithelial Cl− secretion and by inhibiting the epithelial NaCl absorption (Lohi et al. 2002). As previously mentioned, CFTR expression was upregulated in colon (Langmann et al 2004).

FNBP1L: formin binding protein 1-like. FNBP1L protein, through its binding affinities to both CDC42 and N-WASP, which induce actin polymerization, is involved in a pathway that links cell surface signals to the actin cytoskeleton. As mentioned above, FNBP1L expression was upregulated in both colon and ileum (Langmann et al 2004).

KIAA0992: now called PALLD for Palladin. It is a component of actin-containing microfilaments that control cell shape, adhesion, and contraction. Palladin interacts with ezrin, which is essential for epithelial organization and villus morphogenesis in the developing intestine, suggesting an involvement in CD. Palladin is a novel component of stress fiber dense regions and colocalizes and coimmunoprecipitates with alpha-actinin, a dense region component. Palladin expression is up-regulated in differentiating dendritic cells (DCs), and coincides with major cytoskeletal and morphological alterations. In immature DCs, palladin is localized in actin-containing podosomes and in mature DCs along actin filaments. The regulated expression and localization suggest a role for palladin in the assembly of the DC cytoskeleton (Mykkanen, et al., 2001).

NEUROD2: neurogenic differentiation 2. NEUROD2 is a calcium-regulated transcription factor (basic helix-loop-helix (bHLH)) that plays a critical role in regulating synaptic maturation, the patterning of thalamocortical connections, and is essential for amygdala development (Ince-Dunn et al 2006; Lin et al 2005). NEUROD2 also interacts with PKN1, a protein kinase C that mediates the Rho-dependent signaling pathway.

NOS2A: This gene encodes a nitric oxide synthase which is expressed in liver and is inducible by a combination of lipopolysaccharides and certain cytokines. Nitric oxide (NO) is a biological signaling and effector molecule and is especially important during inflammation. In macrophages, NO mediates tumoricidal and bactericidal actions, as indicated by the fact that inhibitors of NO synthase (NOS) block these effects. Neuronal NOS and macrophage NOS are distinct isoforms. Both the neuronal and the macrophage forms are unusual among oxidative enzymes in requiring several electron donors: FAD, FMN, NADPH, and tetrahydrobiopterin. NOS2A expression and nitric oxide (NO) synthesis are increased in epithelial cells and in tissue macrophages of the inflamed mucosa from patients with inflammatory bowel disease (IBD), and it has also been shown that NOS2A is increased in circulating monocytes from patients with active IBD and this increased expression correlates with disease activity (Dijkstra et al 2002). As already mentioned, NOS2A expression was upregulated in sigmoid colon tissue from Crohn's patients compared to control tissue (Costello et al 2005).

POLDIP2: This gene encodes the polymerase (DNA-directed), delta interacting protein 2. This protein interacts with the DNA polymerase delta p50 subunit. The encoded protein also interacts with proliferating cell nuclear antigen (PCNA).

PPM1D: The protein encoded by this gene is a member of the PP2C family of Ser/Thr protein phosphatases (WIP1). PP2C family members are known to be negative regulators of cell stress response pathways. Expression of PPM1D is induced in response to IR in a p53-dependent manner. Wip1 has potential as a drug target, since the work of Bulavin et al. (2004) indicates that inhibition of Wip1 phosphatase can suppress the proliferation of certain types of cancer. Phosphatases are, in principal, susceptible to targeting by drugs, as potent inhibitors of other phosphatases have been developed. The side effects of inhibition of Wip1 might be acceptable, as Wip1-null mice developed normally, even though defects in immune function have been noted (Choi et al., 2002).

PRKCD: The gene PRKCD encodes for the protein kinase C delta protein. Protein kinase C (PKC) is a family of serine- and threonine-specific protein kinases that can be activated by calcium and the second messenger diacylglycerol. Once activated, PKC phosphorylates a range of cellular proteins. PKC family members phosphorylate a wide variety of protein targets and are known to be involved in diverse cellular signaling pathways. PKC family members also serve as major receptors for phorbol esters, a class of tumor promoters. Each member of the PKC family has a specific expression profile and is believed to play distinct roles in cells. The delta polypeptide appears to be the major isoform expressed in mouse hematopoietic cells. Studies both in human and mice demonstrate that PRKCD is involved in B cell signaling and in the regulation of growth, apoptosis, and differentiation of a variety of cell types. Mischak et al. (1991) isolated and characterized the gene encoding the delta polypeptide. PRKCD is involved in B cell signaling and in the regulation of growth, apoptosis, and differentiation of a variety of cell types. PRKCD is most abundant in B and T lymphocytes of lymphoid organs, cerebrum, and the intestine of normal mice. By generating mice with a disruption in the PRKCD gene, Miyamoto et al. (2002) observed that the mice are viable up to 1 year but prone to autoimmune disease, with enlarged lymph nodes and spleens containing numerous germinal centers. PRKCD-deficient B cells also mounted a stronger proliferative response than those from wildtype mice. The importance of PKC-delta in B-cell tolerance is further underscored by the appearance of autoreactive anti-DNA and anti-nuclear antibodies in the serum of PKC-delta-deficient mice. As deficiency of PKC-delta does not affect BCR-mediated B-cell activation in vitro and in vivo, PKC-delta may have a selective and essential role of PKC-delta in tolerogenic, but not immunogenic, B-cell responses. As previously mentioned, PRKCD expression was downregulated in colon (Langmann et al 2004).

RB1CC1: RB1-inducible coiled-coil 1, or focal adhesion kinase (FAK) family interacting protein. RB1CC1 protein binds to the kinase domain of FAK and inhibits its kinase activity and associated cellular functions. It also acts as a putative transcription factor that regulates retinoblastoma 1 (RB1) expression, which is linked to the terminal differentiation of many tissues and cells (Kontani et al 2003). RB1CC1 inhibits G1-S phase progression, proliferation, and clonogenic survival in human breast cancer cells (Melkoumian et al 2005). By interacting with the TSC1-TSC2 complex, RB1CC1 also has a cellular function in the regulation of cell size (Gan et al 2005). RB1CC1 is abundantly expressed in human musculoskeletal cells and is expression is a prerequisite for myogenic differentiation.

RPS6KB1: This gene encodes a member of the RSK (ribosomal S6 kinase) family of serine/threonine kinases. This kinase contains 2 non-identical kinase catalytic domains and phosphorylates several residues of the S6 ribosomal protein. The kinase activity of this protein leads to an increase in protein synthesis and cell proliferation. Amplification of the region of DNA encoding this gene and overexpression of this kinase are seen in some breast cancer cell lines.

SALL1: sal-like 1 (Drosophila). The Spalt (sal) gene family plays an important role in regulating developmental processes of many organisms. SALL1 is a zinc-finger nuclear factor that acts as a strong transcriptional repressor in mammalian cells. Mutations in the SALL1 gene cause an autosomal dominantly inherited disorder (Townes-Brocks syndrome) characterized by typical malformations of the thumbs, the ears, and the anus, and also commonly affect the kidneys development and other organ systems. SALL1 seems to be involved in the regulation of higher order chromatin structures which indicates that the protein might be a component of a distinct heterochromatin-dependent silencing process (Netzer et at 2001). SALL1 also seems to activate canonical Wnt signaling (Sato et al 2004). Interestingly, Wnt signaling seems to have an essential role in the maintenance of adult small intestine and colon proliferation. Hence, potential clinical applications in mucosal repair for inflammatory bowel diseases have been suggested (Hoffman et al 2004).

WWOX: This WW domain containing oxidoreductase (WWOX), contains 2 WW domains coupled to a region with high homology to the short-chain dehydrogenase/reductase (SRD) family of enzymes. WW domain-containing proteins are found in all eukaryotes and play an important role in the regulation of a wide variety of cellular functions such as protein degradation, transcription, and RNA splicing. The encoded protein is more than 90% identical to the mouse Wox1 protein, which is an essential mediator of tumor necrosis factor-alpha-induced apoptosis, suggesting a similar, important role in apoptosis for the human protein (Chang et at 2001). In addition, there is evidence that this gene behaves as a suppressor of tumor growth.

Network 6 Direct Only

Network 6 contains 44 nodes (35 original and 9 manual additions) with 12 genes from the fine mapped regions (FIG. 6). A short description of these 12 genes follows. From their role in immune response and/or inflammation (CAPZD, CSF3, IL4, NLK), biochemical pathways involved in the disease pathogenesis (RHOA), some of these genes are very good candidates to play a role in CD (see below).

The expression of 10 genes of this network has been shown to vary in some studies of gene expression profiling for CD. In sigmoid colon tissue from Crohn's patients compared to control tissue, NLK was upregulated and DVL2, ROCK1, CSDE1 were downregulated (Costello et at 2005). In the study by Langmann et at 2004, PPP1R1B has been shown to be downregulated in ileum of Crohn's patient compared to control specimens; ACTN4 was downregulated in both colon and ileum; TPM1, NLK, CTNNB1, HSPA1A, and CEBPB were upregulated in colon.

CAPZB: capping protein (actin filament) muscle Z-line, beta, or F-actin capping protein beta subunit. As a member of the F-actin capping protein family, CAPZB regulates actin filament assembly and organization by capping the barbed end of growing actin filaments. CAPZB is important in T cell signaling (Hutchings et al 2003).

CSF3: CSF3 colony stimulating factor 3 (granulocyte) is a hematopoietic cytokine that is important for allergic inflammation. Delineating the biology of these cytokines is enabling the development of new strategies for diagnosing and treating diseases and modulating immune responses. The protein encoded by this gene is a cytokine that controls the production, differentiation, and function of granulocytes. The active protein is found extracellularly. Three transcript variants encoding three different isoforms have been found for this gene. Granulocyte colony-stimulating factor (or colony stimulating factor-3) specifically stimulates the proliferation and differentiation of the progenitor cells for granulocytes (Metcalf, 1985). Harada et al. (2005) suggested that CSF3 promotes survival of cardiac myocytes and prevents left ventricular remodeling after myocardial infarction through functional communication between cardiomyocytes and noncardiomyoctes. Disseminated colon cancer exhibits severe peripheral blood eosinophilia and elevated serum levels of interleukin-2, interleukin-3, interleukin-5, and GM-CSF.

IL4: IL4, interleukin 4, is also known as B cell stimulatory factor 1 (BSF1) and lymphocyte stimulatory factor 1. The IL-4 receptor is expressed ubiquitously on monocytes and macrophages, and as with other class I cytokine receptors (hematopoietin receptor family), the IL-4 receptor lacks intrinsic kinase activity and requires receptor-associated kinases for initiation of intracellular signaling. Binding of IL-4 to its receptor leads to JAK1 and JAK3 activation. IL-4 is a potent anti-inflammatory cytokine and leads to an “alternative activation phenotype” in macrophages. This IL-4-dependent macrophage activation results in an absence of nitric oxide production, generation of IL-10 and IL-1 receptor antagonist, and suppressive activity directed toward T cells (Hartman et al., 2004). This gene has been associated with CD but not ulcerative colitis in 2 studies out of 2 (Aithal 2001; Klein 2001).

KIF3A: kinesin family member 3A. The KIF3A/KIF3B heterodimer functions as a new microtubule-based anterograde translocator for membranous organelles who plays an important role not only in interphase but also in mitosis. The MAP kinase kinase kinases MLK2 and MLK3 interact with members of the KIF3 family of kinesin superfamily motor proteins and with KAP3A, the putative targeting component of KIF3 motor complexes, suggesting a potential link between stress activation and motor protein function (Nagata et al. 1998). This protein interacts with PPP1R15A, one of a subset of proteins induced after DNA damage or cell growth arrest (Hasegawa et al., 2000).

NLK: This gene encodes the Nemo like kinase. Recent searches revealed that nemo-like kinase (NLK) was a negative regulator of the wingless type (wnt) signal cascade in Xenopus and Caenorhabditis elegans. NLK phosphorylates T-cell factor (TCF)/lymphoid enhancer-binding factor (LEF) and interferes with binding of the beta-catenin-TCF/LEF complex to its TCF target site. Interestingly, NLK has recently been shown to interact with STAT3 (Kojima et al 2005), and STAT3 has been involved in the pathogenesis of CD. In colon tissue from Crohn's patients compared to control tissue, NLK expression was upregulated (Langmann et al 2004; Costello et at 2005).

PAI-RBP1: now called SERBP1. This gene encodes a SERPINE1 mRNA binding protein 1. Interestingly, the 4G/4G genotype of the 4G/5G polymorphism of the type-1 plasminogen activator inhibitor (PAI-1 or SERPIN1) gene is a determinant of penetrating behaviour in patients with Crohn's disease (Sans et al 2003).

PPP1R1B: protein phosphatase 1, regulatory (inhibitor) subunit 1B (dopamine and cAMP regulated phosphoprotein, DARPP-32). Glutamatergic receptor stimulation elevates intracellular calcium, which leads to activation of calcineurin and dephosphorylation of phospho-DARPP32, thereby reducing the phosphatase-1 inhibitory activity of DARPP32. The expression of DARPP32 is associated with a potent antiapoptotic advantage for gastric cancer cells through a p53-independent mechanism that involves preservation of mitochondrial potential and increased Bcl2 levels (Belkhiri, 2005). In the study by Langmann et al 2004, PPP1R1B expression was downregulated in ilea of Crohn's patients compared to control specimens.

PRDX3: peroxiredoxin 3. This gene encodes a protein with antioxidant function and is localized in the mitochondrion. Wonsey et al (2002) have shown that PRDX3 is required for Myc-mediated proliferation, transformation, and apoptosis after glucose withdrawal, and that this protein is required to maintain normal mitochondrial function.

RHOA: RhoA is a member of the Rho subfamily of small GTPases and has been shown to be involved in a diverse set of signaling pathways including the ultimate regulation of the dynamic organization of the cytoskeleton. Increased activation of RhoA was found in inflamed intestinal mucosa of patients with Crohn's disease and in rats with 2,4,6-trinitrobenzene sulfonic acid-induced colitis. In vitro, activation of RhoA alone was sufficient to induce tumor necrosis factor production. Rho kinase inhibition prevents nuclear factor kappa B activation and I-kappa B phosphorylation and degradation. Rho kinase activates I-kappa B kinase and, thus, nuclear factor kappa B, suggesting a key role of Rho kinase in inflammatory responses and intestinal inflammation. Specific inhibition of Rho kinase may be a promising approach for the treatment of patients with Crohn's disease (Segain et al., 2003).

SERPINC1: serine (or cysteine) proteinase inhibitor, clade C (antithrombin), member 1, is the most important serine protease inhibitor in plasma that regulates the blood coagulation cascade. SERPINC1 inhibits thrombin as well as factors IXa, Xa and XIa. Mutations in SERPINC1 were previously shown to be associated with thrombosis and such mutations are exacerbated by infection. Thrombin-antithrombin III complexes are increased in inflammatory bowel diseases and suggest that thrombin generation might be an early event in their pathogenesis (Chamouard et al 1995).

TSN: The translin gene encodes a DNA-binding protein which specifically recognizes conserved target sequences at the breakpoint junction of chromosomal translocations. In their study, Aoki et al (1995) showed that nuclear localization of TSN is limited to lymphoid cell lines, and they hypothesized that nuclear transport of this protein is regulated in a physiologically significant way such that active nuclear transport is associated with the lymphoid specific process known as Ig/TCR gene rearrangement.

VTN: This gene encodes vitronectin which is a cell adhesion and spreading factor found in serum and tissues. Vitronectin interacts with glycosaminoglycans and proteoglycans. It is recognized by certain members of the integrin family and serves as a cell-to-substrate adhesion molecule. It is a secreted protein and exists in either a single chain form or a clipped, two chain form held together by a disulfide bond. It has been implicated as a participant in diverse biological processes, including cell attachment and spreading, complement activation, and regulation of hemostasis.

Network 7 Direct Only

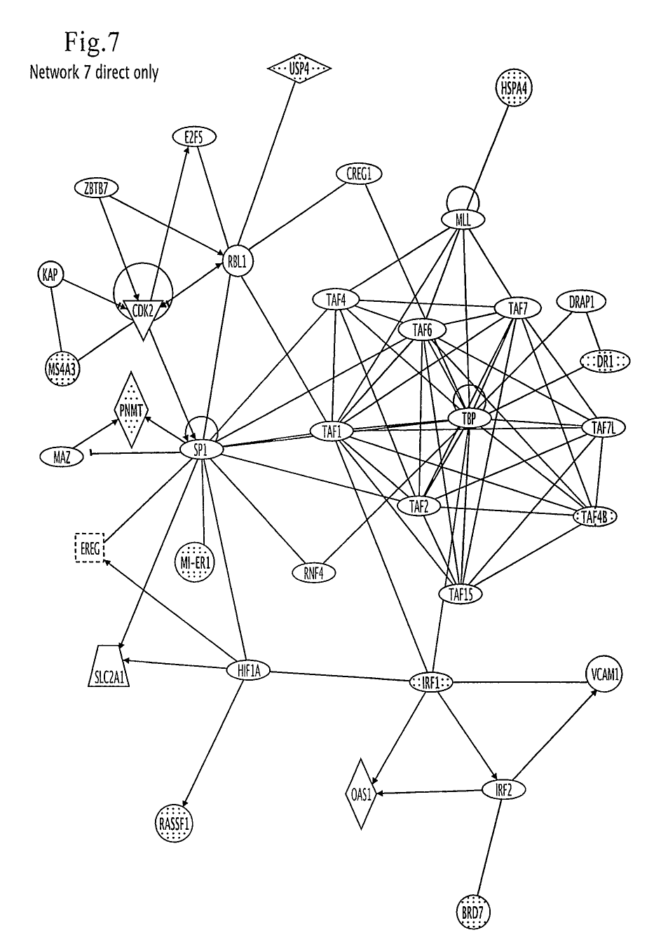

Network 7 contains 35 original nodes with 10 genes from the fine mapped regions (FIG. 7). A short description of these 10 genes follows. From their role in immune response and/or inflammation (IRF1, MS4A3, TAF4B, USP4), barrier integrity, protection, and function (BRD7, HSPA4), gastrointestinal physiology (PNMT), some of these genes are very good candidates to play a role in CD (see below). The expression of 9 genes of this network has been shown to vary in some studies of gene expression profiling for CD. In sigmoid colon tissue from Crohn's patients compared to control tissue, TAF4 was upregulated (Costello et al 2005). In the study by Langmann et al 2004, OAS1 was shown to be downregulated in ileum of Crohn's patient compared to control specimens; DRAP1 was downregulated in colon; EREG and MLL were upregulated in both colon and ileum; VCAM1, CREG1, TAF7, and DR1 were upregulated in colon.

BRD7: bromodomain containing 7. The bromodomain is a 110 amino acid evolutionally conserved domain and is found in proteins strongly implicated in signal-dependent transcriptional regulation. BRD7 has been shown to inhibit cell growth and cell cycle progression, and may present a new associated tumor suppressor gene (Zhou et at 2004). BRD7 has also been shown to play a regulatory role in Wnt signaling (Kim et al 2003). As mentioned in the description of the SALL1 gene, the Wnt signaling pathway has an essential role in the maintenance of adult small intestine and colon proliferation (Hoffman et at 2004).

DR1: down-regulator of transcription 1, TBP-binding (negative cofactor 2). This gene encodes a TBP- (TATA box-binding protein) associated phosphoprotein that represses both basal and activated levels of transcription. In vivo phosphorylation of the protein affects its interaction with TBP (Inostroza et at 1992). As mentioned above, expression of DR1 was upregulated in colon (Langmann et al 2004).

HSPA4: heat shock 70 kDa protein 4. Heat shock proteins (HSP) are evolutionarily conserved stress proteins which protect cell against stress and injury. The Hsp70 family plays important roles in intracellular trafficking and conformation of proteins by acting as a molecular chaperon. Some reports have suggested the protective role of inducible Hsp70 in intestinal cells: these proteins could help in maintenance of intestinal epithelial cell structure and function, and in reducing or alleviating mucosal injury, thereby promoting tissue healing and repair during intestinal inflammation. Indeed, a study reported that heat shock treatment of mice prevents high production of interleukin-6 and nitric oxide and reduces severe damage and apoptosis of the enterocytes in the bowel. However, mice deficient in the inducible hsp70-1 gene were no longer protected by the heat shock treatment (Van Molle et al 2002). Also, two studies looked for genetic associations between inducible Hsp70-2 gene variant, another member of the family, and CD. They showed that allele A of the Hsp70-2 gene was associated with a less severe form of CD (Esaki et al 1999; Klausz et al 2005). Recently, a downregulation of heat shock 70-kDa protein 4 was reported after MDP (the bacterial proteoglycan fragment muramyl dipeptide, a ligand for NOD2) stimulation of NOD2-overexpressing human cultured cells (Weichart et al 2006).

IRF1: IRF1 encodes interferon regulatory factor 1, a member of the interferon regulatory transcription factor (IRF) family. IRF1 serves as an activator of interferons alpha and beta transcription. Further, IRF1 has been shown to play a role in regulating apoptosis and tumor-suppression. An increased expression of IRF1 in lamina propria mononuclear cells from patients with Crohn's disease has been described and may be relevant to the pathogenesis of CD (Clavell et al 2000). Interestingly, an IRF1 binding motif is present in the promoter of the CARD4/NOD1 gene, and this binding site has been shown to be essential for the increase in gene and protein expression induced by IFN gamma (Hisamatsu et al 2003).

MI-ER1: now called MIER1. This gene is a mesoderm induction early response 1 homolog and has a transcription regulatory activity. This gene encodes a protein that functions as transcriptional repressor by recruitment of histone deacetylase 1 (Ding et al 2003).

MS4A3: membrane-spanning 4-domains, subfamily A, member 3 (hematopoietic cell-specific). This gene encodes a member of a gene family which is characterized by common structural features and similar intron/exon splice boundaries and displays unique expression patterns among hematopoietic cells and nonlymphoid tissues. MS4A3 likely plays a role in signal transduction and may function as a subunit associated with receptor complexes. MS4A3 expression is tightly regulated during the differentiation of hematopoietic stem cells, and this protein functions as a hematopoietic cell cycle regulator (Donato et al 2002).

PNMT: phenylethanolamine N-methyltransferase. This enzyme catalyzes the synthesis of epinephrine from norepinephrine, the last step of catecholamine biosynthesis. The role of sympathetic regulation of gastrointestinal physiology is known. Recently, a role for the sympathetic microenvironment in regulation of colonic macrophage TNF and IL-6 secretion has been described (Straub et al 2005).

RASSF1: Ras association (RaIGDS/AF-6) domain family 1. This gene encodes a protein similar to the RAS effector proteins. Loss or altered expression of this gene has been associated with the pathogenesis of a variety of cancers, which suggests the tumor suppressor function of this gene (Shivakumar et al 2002).

TAF4B: This gene encodes a TAF4b RNA polymerase II, TATA box binding protein (TBP)-associated factor, 105 kDa. TATA-binding protein associated factors (TAFs) participate, with TATA binding protein, in the formation of the TFIID protein complex, which is involved in the initiation of gene transcription by RNA polymerase II. Interestingly, Yamit-Hezi et al (1998) have shown that TAFII105 mediates activation of anti-apoptotic genes by NF-kappaB. In addition, it has been shown that TAF(II)105 has a pro-survival role in B and T lymphocytes, where the native protein is expressed. In addition, TAF(II)105 is important for T cell maturation and for production of certain antibody isotypes (Silkov et al 2002). For these reasons, the TAF4B gene is a good candidate gene to play a role in CD.

USP4: This gene encodes an ubiquitin specific peptidase 4 which has been shown to specifically interact with the retinoblastoma protein (DeSalle et al 2001). Recently, USP4 has been found to be an interaction partner for the carboxyl-terminal tail of the GPCR adenosine A2A receptor (ADORA2A). The binding of USP4 to ADORA2A allows for its accumulation as a deubiquinated protein. This relaxes ER quality control and enhances cell surface expression of functionally active receptors, which are otherwise predominantly intracellular (Milojevic et al 2006). This is particularly interesting because activation of ADORA2A by the selective A2A agonist ATL-146e has been reported to reduce intestinal inflammation in animal models of inflammatory bowel disease (Odashima et at 2005).

Networks 8 to 23 Direct Only

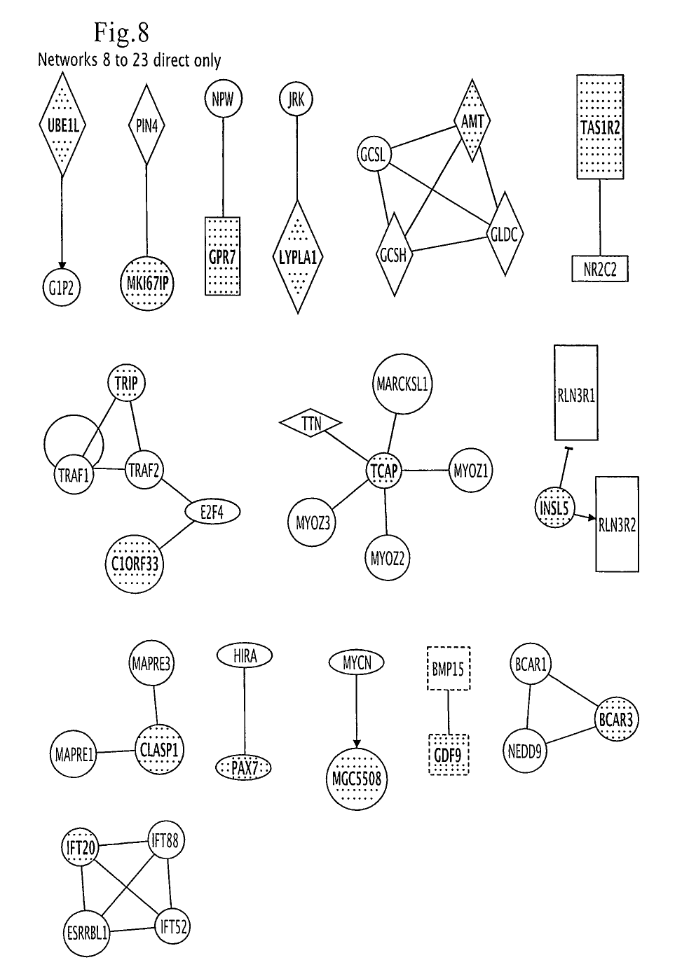

Networks 8 to 23 contain 44 original nodes with 16 genes from the fine mapped regions (FIG. 8). From their role in immune response and/or inflammation (TRIP), biochemical pathways involved in the disease pathogenesis (C1orf33, GDF9), some of these genes are very good candidates to play a role in CD (see below). The expression of 6 genes from these networks has been shown to vary in some studies of gene expression profiling for CD. In sigmoid colon tissue from Crohn's patients compared to control tissue, GCSH and NEDD9 were downregulated (Costello et al 2005). In the study by Langmann et at 2004, LYPLA1, MYCN, and ESRRBL1 have been shown to be upregulated in ilea of Crohn's patients compared to control specimens; GLDC was upregulated in colon.

AMT: This gene encodes an aminomethyltransferase (also called glycine cleavage system protein T), which is an important enzyme in glycine metabolism. Missense mutations in the T-protein gene have been associated with nonketotic hyperglycinemia (Nanao et al 1994; Kure et al 1998).

BCAR3: breast cancer anti-estrogen resistance 3. BCAR3 was identified in the search for genes involved in the development of estrogen resistance. The gene encodes a component of intracellular signal transduction that causes estrogen-independent proliferation in human breast cancer cells. AND-34, a novel GDP exchange factor, is a murine homolog of human BCAR3. AND-34 is expressed constitutively at significant levels in murine splenic B cells, but not in murine splenic T cells or thymocytes, and AND-34 has also been shown to be regulated by inflammatory cytokines IL1 and TNF (Cai et al 1999; Cai et at 2003).

C1orf33: This gene encodes a protein sharing a low level of sequence similarity with ribosomal protein P0. The exact function of the encoded protein is currently unknown. However, E2F4 has been shown to bind the endogenous promoter of C1orf33 (Ren et at 2002). E2F-4 is a transcription factor involved in the transition of the cell from the resting state (G0/G1) to the proliferative stage (S). A study reported that the nucleocytoplasmic shuttling of E2F4 was involved in the regulation of human intestinal epithelial cell proliferation and differentiation (Deschenes et at 2004). In addition, E2F4 was expressed in colorectal adenocarcinoma (Mady et at 2002). Also, activation of human PPARG protein decreases expression of human C1orf33 mRNA (Sarraf et at 1998). Since PPARG has been identified as a susceptibility gene in both the SAMP/Yit mouse and in human Crohn's disease (Sugawara et at 2005), C1orf33 is a good candidate to play a role in CD pathogenesis, even if its exact role is not yet known.

CLASP1: CLASP1 is a nonmotor microtubule-associated protein and is involved in the regulation of microtubule dynamics at the kinetochore and throughout the spindle. CLASP1 interacts with MAPRE1 microtubule-associated protein, RP/EB family, member 1, and with MAPRE3. MAPRE1 and 3 are members of the RP/EB family of genes. MAPRE1 can bind the APC protein which is often mutated in familial and sporadic forms of colorectal cancer. MAPRE1 protein localizes to microtubules, especially the growing ends, in interphase cells. During mitosis, the protein is associated with centrosomes and spindle microtubules. The protein also associates with components of the dynactin complex and the intermediate chain of cytoplasmic dynein. Because of these associations, it is thought that this protein is involved in the regulation of microtubule structures and chromosome stability. MAPRE3 localizes to the cytoplasmic microtubule network.

GDF9: growth differentiation factor 9. GDF9 is a member of the transforming growth factor-beta superfamily, is expressed in oocytes and is thought to be required for ovarian folliculogenesis (Aaltonen et al 1999). Bone morphogenetic protein receptor type II is a receptor for GDF9 (Vitt et al 2002). Interestingly, BMPR II is expressed in colonic epithelial cell lines. In vitro, BMP2 inhibits colonic epithelial cell growth by promoting apoptosis and differentiation and inhibiting proliferation. Among others, BMP2 and BMPRII are expressed predominantly in mature colonocytes at the epithelial surface in normal adult human and mouse colon. BMP2 expression is lost in the microadenomas of familial adenomatous polyposis patients. These results suggest that BMP2 acts as a tumor suppressor promoting apoptosis in mature colonic epithelial cells (Hardwick et al 2004).

GPR7: now called NPBWR1. GPR7 is an orphan G protein-coupled receptor, presenting high similarities with opioid and somatostatin receptors. The human 7-transmembrane receptor GPR7 can be activated by the recently discovered neuropeptides NPB and NPW. GPCRs are in general excellent drug targets. Data indicate that NPB and NPW signaling via the GPR7 receptor plays a biologically important role in regulating food intake, energy expenditure, and body weight (Ishii et al, 2003). G Protein Receptors 7 and 8 are expressed in human adrenocortical cells, and their endogenous ligands, neuropeptides B and W, enhance cortisol secretion by activating adenylate cyclase- and phospholipase C-dependent signaling cascades (Mazzocchi G. et al, 2005). This receptor is highly expressed in the nervous system, with suggested roles in neuroendocrine events and pain signaling. There is a relationship between the pathogenesis of inflammatory/immune-mediated neuropathies, GPR7 receptor expression, and pain transmission (Zaratin PF et al, 2005).

IFT20: This gene encodes an intraflagellar transport 20 homolog. Intraflagellar transport is very important in assembling and maintaining many cilia/flagella, and is involved in many processes such as the motile cilia that drive the swimming of cells, the generation of left-right asymmetry in vertebrate embryos, and the detection of some sensory stimuli (Yin et al 2003).

INSL5: insulin-like 5. INSL5 is a peptide that belongs to the relaxin/insulin family. INSL5 is a high affinity specific agonist for GPCR142 (GPR100, Liu et al 2005).

LYPLA1: lysophospholipase I. Lysophospholipases are enzymes that act on biological membranes to regulate the multifunctional lysophospholipids. The protein encoded by this gene hydrolyzes lysophosphatidylcholine in both monomeric and micellar forms. As already mentioned, LYPLA1 expression has been shown to be upregulated in ileum of Crohn's patient compared to control specimens (Langmann et al 2004).

MGC5508: now called TMEM109. The function of this protein is unknown.

PAX7: paired box gene 7. This gene is a member of the paired box (PAX) family of transcription factors. These genes play critical roles during fetal development and cancer growth. The specific function of PAX7 is unknown.

TAS1R2: taste receptor, type 1, member 2. Mouse T1 r2 and T1 r3 combine to function as a receptor recognizing sweet-tasting molecules as diverse as sucrose, saccharin, dulcin, and acesulfame-K.

TCAP: titin-cap (telethonin). Sarcomere assembly is regulated by the muscle protein titin. This gene encodes a protein found in striated and cardiac muscle that binds to the titin Z1-Z2 domains and is a substrate of titin kinase, interactions thought to be critical to sarcomere assembly. Mutations in this gene are associated with limb-girdle muscular dystrophy type 2G (Moreira et al 2000).

TRIP: The TRIP gene, now called TRAIP, and also known as TRAF interacting protein, encodes a protein that contains an N-terminal RING finger motif and a putative coiled-coil domain. Tumor necrosis factor (TNF) receptor associated factors (TRAFs) were first identified as two intracellular proteins, TRAF1 and TRAF2. Many of the biological effect of TRAF signaling are mediated by the activation of kinases such as the IkB kinase (IKK) and mitogen-activated protein (MAP) kinases, which in turn modulate the transcriptional activities of the NF-kB and AP-1 families respectively. Thus this gene could be involved in the inflammatory processes seen in CD.

UBE1L: this gene encodes an ubiquitin-activating enzyme E1-like. This gene encodes a member of the E1 ubiquitin-activating enzyme family. The encoded enzyme is a retinoid target that triggers promyelocytic leukemia (PML)/retinoic acid receptor alpha (RARalpha) degradation and apoptosis in acute promyelocytic leukemia.The modification of proteins with ubiquitin is an important cellular mechanism for targeting abnormal or short-lived proteins for degradation.

MKI67IP: MKI67 (FHA domain) interacting nucleolar phosphoprotein. MKI67IP is a nucleolar protein interacting with the FHA domain of pKi-67, a large nuclear protein associated with the cell-cycle.

### Results from Analyses Incorporating both Direct and Indirect Interactions for 295 Annotated Genes

Network 1b Direct and Indirect

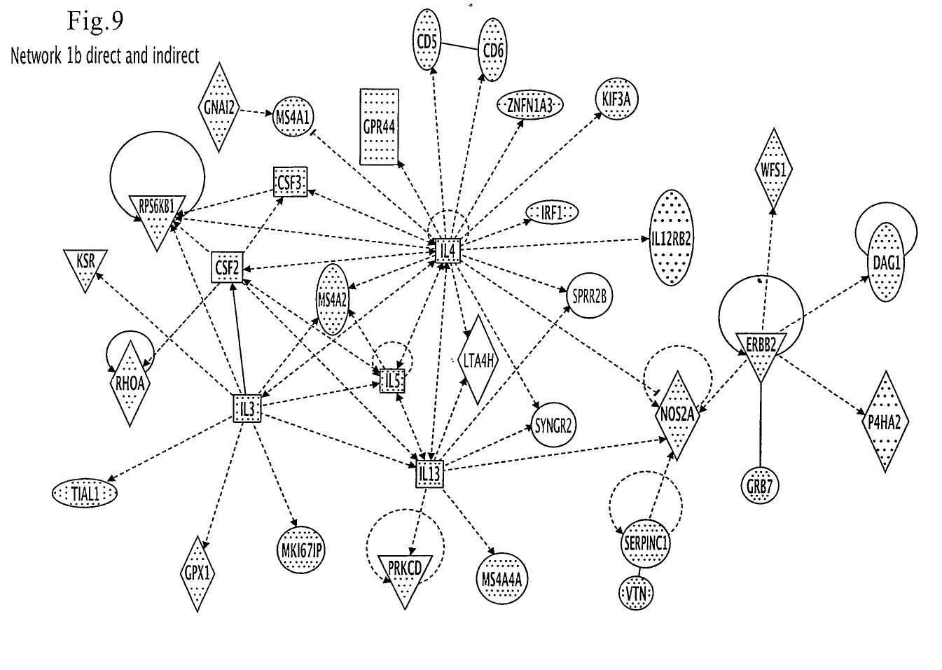

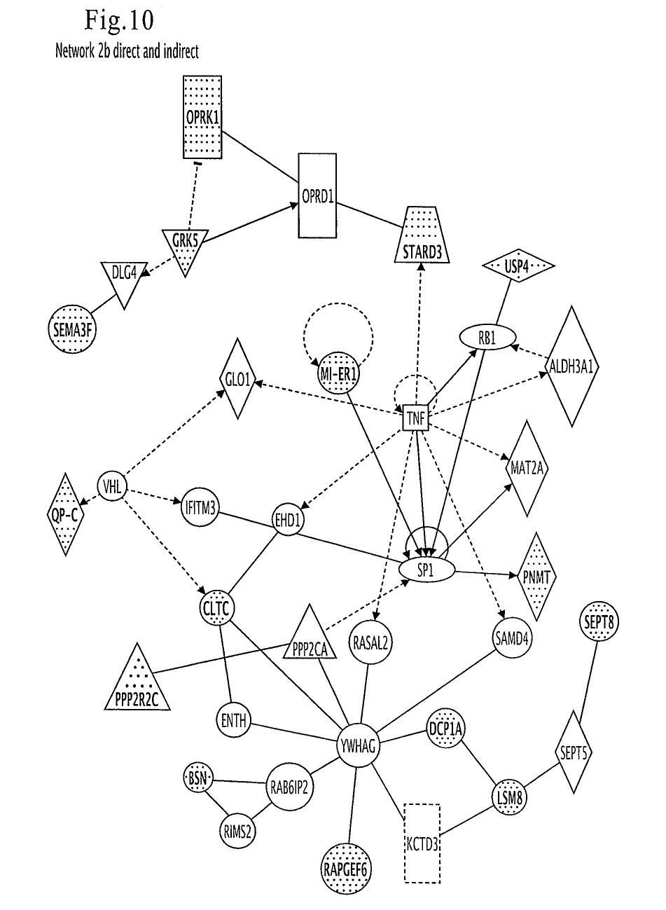

The network 1b contains 35 original nodes with 32 genes from the fine mapped regions (FIG. 9). The expression of 4 genes of this network has been shown to vary in some studies of gene expression profiling for CD. In sigmoid colon tissue from Crohn's patients compared to control tissue, NOS2A and SYNGR2 were upregulated (Costello et at 2005). In the study by Langmann et al 2004, PRKCD has been shown to be downregulated in colons of Crohn's patients compared to control specimens; MS4A1 was downregulated in both colon and ileum.

For genes CD5, CD6, CSF2, CSF3, DAG1, ERBB2, GNAI2, GPR44, GPX1, GRB7, IL3, IL4, IL5, IL12RB2, IL13, IRF1, KIF3A, KSR1, MKI67IP, MS4A1, MS4A2, NOS2A, PRKCD, RHOA, RPS6KB1, SERPINC1, VTN, ZNFN1A3: please refer to descriptions included in the networks from direct analysis only.

MS4A4A: membrane-spanning 4-domains, subfamily A, member 4. This gene encodes a member of the membrane-spanning 4A gene family. Members of this nascent protein family are characterized by common structural features and similar intron/exon splice boundaries and display unique expression patterns among hematopoietic cells and nonlymphoid tissues.

TIAL1: TIA1 cytotoxic granule-associated RNA binding protein-like 1. The protein encoded by this gene is a member of a family of RNA-binding proteins, has three RNA recognition motifs (RRMs), and binds adenine and uridine-rich elements in mRNA and pre-mRNAs of a wide range of genes. It regulates various activities including translational control, splicing and apoptosis. TIAR displays a high affinity binding to the human NOS2A 3′-UTR sequence, and seems to be involved in the post-transcriptional regulation of human iNOS expression (Fechir et al 2005). As described in network 5 direct only, NOS2A expression and nitric oxide (NO) synthesis are increased in epithelial cells and in tissue macrophages of the inflamed mucosa from patients with inflammatory bowel disease (IBD), and NOS2A is increased in circulating monocytes from patients with active IBD and this increased expression correlates with disease activity (Dijkstra et al 2002).

In addition, TIAR isoform plays a major role in the post-transcriptional silencing for human matrix metalloproteinases-13 (MMP13). MMP13 shows a wide substrate specificity, and its expression is limited to pathological situations such as chronic inflammation and cancer (Yu et al 2003).

WFS1: Wolfram syndrome 1 (woiframin). WFS1 is a transmembrane protein. Mutations in this gene are associated with Wolfram syndrome (Strom et at 1998). Wolfram syndrome is an autosomal recessive syndrome characterized by insulin-dependent diabetes mellitus and bilateral progressive optic atrophy, usually presenting in childhood or early adult life. Diverse neurologic symptoms, including a predisposition to psychiatric illness, may also be associated with this disorder. Mutations in this gene can also cause autosomal dominant deafness 6 (DFNA6) (Bespalova et al 2001).

Network 2b Direct and Indirect

The network 2b contains 35 original nodes with 15 genes from the fine mapped regions (FIG. 10). The expression of 6 genes of this network has been shown to vary in some studies of gene expression profiling for CD. In sigmoid colon tissue from Crohn's patients compared to control tissue, CLTC was upregulated (Costello et al 2005). In the study by Langmann et al 2004, ENTH has been shown to be downregulated in ilea of Crohn's patients compared to control specimens; RB1 was upregulated in both colon and ileum; KCTD3 was upregulated in ileum; GRK5 was upregulated in colon. Contradictory results have been found for PPP2CA: it has been shown to be downregulated in colon in the study of Costello et al 2005, and upregulated in colon and ileum in the study of Langmann et al 2004.

For genes BSN, CLTC, DCP1A, GRK5, LSM8, MI-ER1, OPRK1, PNMT, PPP2R2C, RAPGEF6, SEMA3F, SEPT8, STARD3, USP4: please refer to descriptions included in the networks from direct analysis only.

QP-C: now called UQCRQ. ubiquinol-cytochrome c reductase, complex III subunit VII, 9.5 kDa. This gene encodes a ubiquinone-binding protein. This protein is a small core-associated protein and a subunit of ubiquinol-cytochrome c reductase complex III, which is part of the mitochondrial respiratory chain.

Network 3b Direct and Indirect

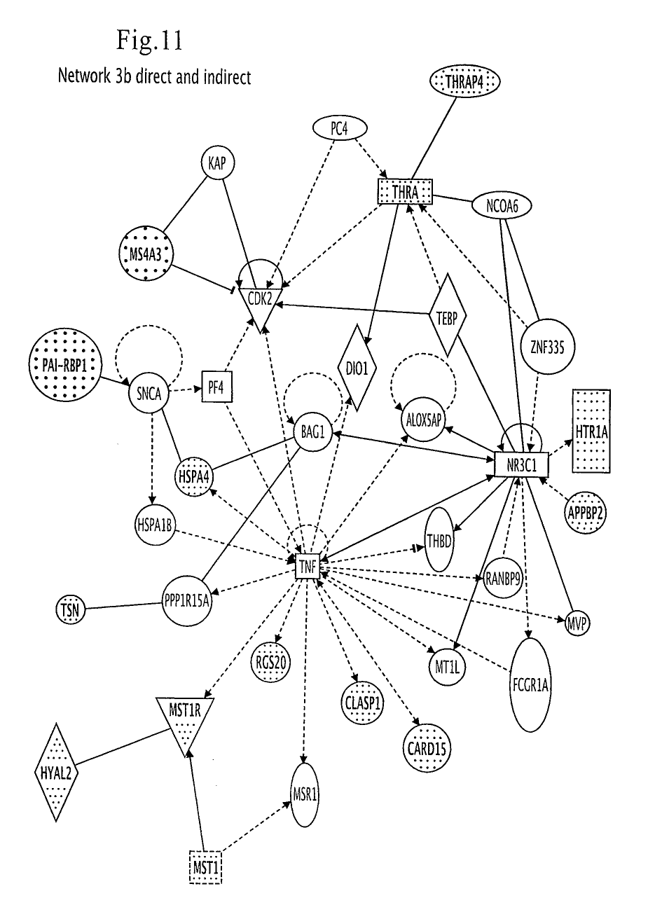

Network 3b contains 35 original nodes with 14 genes from the fine mapped regions (FIG. 11). The expression of 12 genes of this network has been shown to vary in some studies of gene expression profiling for CD. In sigmoid colon tissue from Crohn's patients compared to control tissue, ALOX5AP was upregulated (Costello et al 2005). In the study by Langmann et al 2004, PC4 and NR3C1 have been shown to be downregulated in ilea of Crohn's patients compared to control specimens; MST1 was upregulated in ileum; APPBP2 was upregulated in colon; THRAP4, BAG1, RANBP9, MST1R, and HYAL2 were downregulated in colon; HSPA1B was upregulated in both colon and ileum. Contradictory results have been found for SNCA: it has been shown to be downregulated in colon in the study of Costello et al 2005, and upregulated in colon and ileum in the study of Langmann et al 2004.

For the 14 genes from fine mapped regions in this network: please refer to descriptions included in the networks from direct analysis only.

Network 4b Direct and Indirect

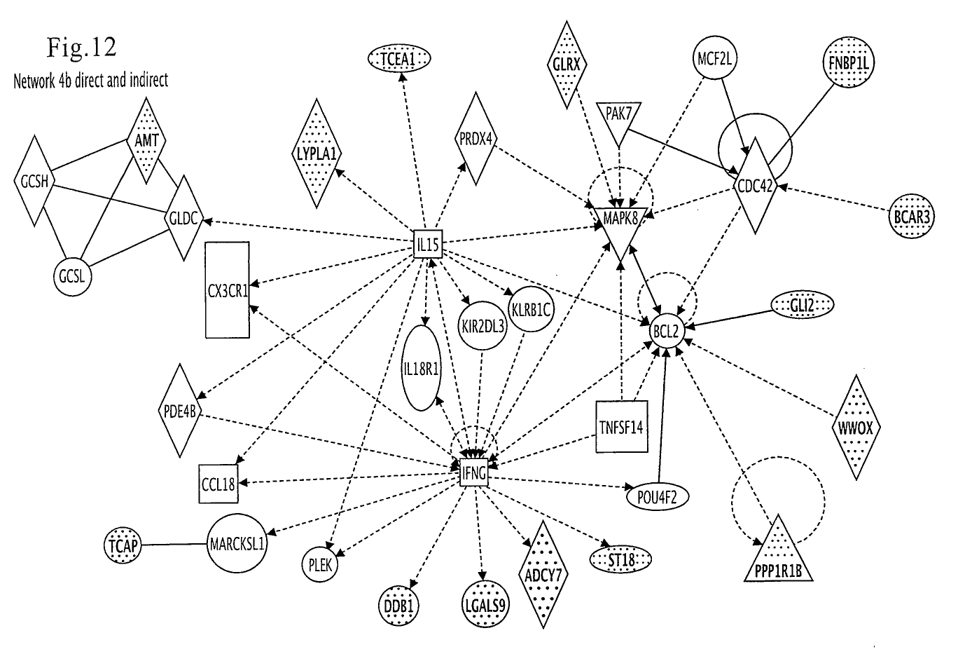

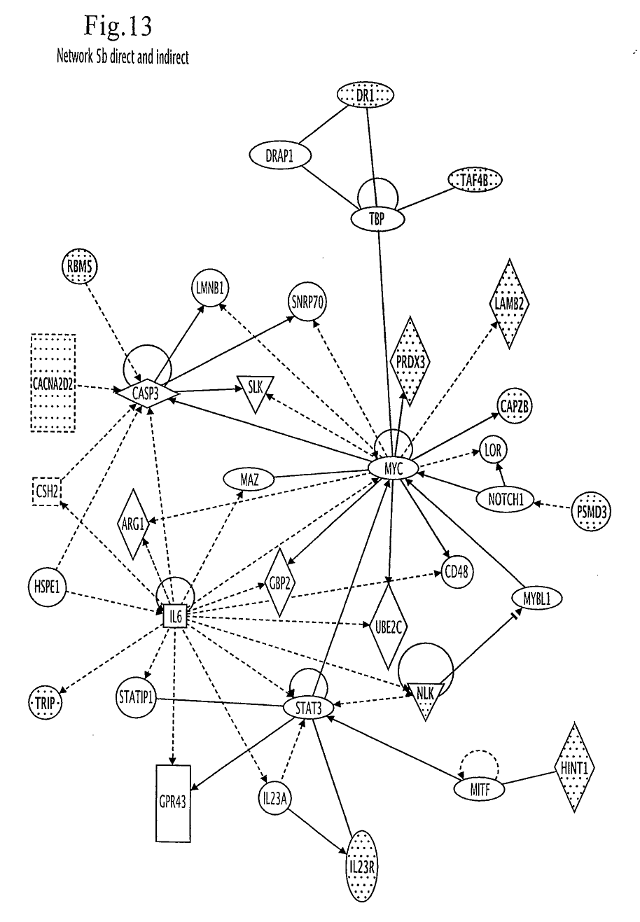

Network 4b contains 35 original nodes with 14 genes from the fine mapped regions (FIG. 12). The expression of 11 genes of this network has been shown to vary in some studies of gene expression profiling for CD. In sigmoid colon tissue from Crohn's patients compared to control tissue, GCSH was upregulated (Costello et al 2005). In the study by Langmann et al 2004, CCL18, PLEK, PPP1 R1 B have been shown to be downregulated in ileum of Crohn's patient compared to control specimens; LYPLA1 was upregulated in ileum; GLI2, GLDC, and PDE4B were upregulated in colon; CDC42 was downregulated in colon; WWOX and FNBP1L were upregulated in both colon and ileum; LGALS9 was downregulated in both colon and ileum.

For the 14 genes from fine mapped regions in this network: please refer to descriptions included in the networks from direct analysis only.

Network 5b Direct and Indirect

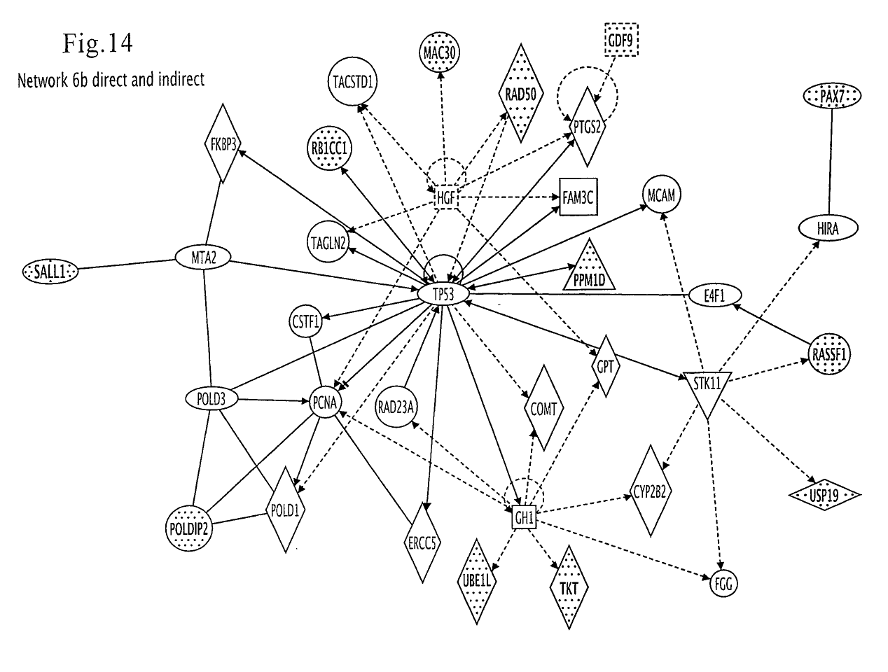

Network 5b contains 35 original nodes with 12 genes from the fine mapped regions (FIG. 13). The expression of 12 genes of this network has been shown to vary in some studies of gene expression profiling for CD. In sigmoid colon tissue from Crohn's patients compared to control tissue, NLK and PSMD3 were upregulated (Costello et al 2005). In the study by Langmann et al 2004, DRAP1, SNRP70, LAMB2, and PSMD3 were downregulated in colon; GBP2 was upregulated in both colon and ileum; DR1, LMNB1, ARG1, NLK and MITF were upregulated in colon.

For genes CAPZB, DR1, HINT1, IL23R, LAMB2, NLK, PRDX3, PSMD3, TAF4B, TRIP: please refer to descriptions included in the networks from direct analysis only.

CACNA2D2: calcium channel, voltage-dependent, alpha 2/delta subunit 2. This gene encodes a member of the alpha-2/delta subunit family, a protein in the voltage-dependent calcium channel complex. Calcium channels mediate the influx of calcium ions into the cell upon membrane polarization and consist of a complex of alpha-1, alpha-2/delta, beta, and gamma subunits in a 1:1:1:1 ratio. A mutation in Cacna2d2, the gene encoding the alpha 2 delta-2 voltage-dependent calcium channel accessory subunit, has been found to underlie the ducky phenotype in mouse (a model for absence epilepsy), which is characterized by spike-wave seizures and cerebellar ataxia (Brodbeck et al 2002).

RBM5: RNA binding motif protein 5. RBM5 is a known modulator of apoptosis (in T cells and other types of cells), an RNA binding protein, and a putative tumor suppressor.

Network 6b Direct and Indirect

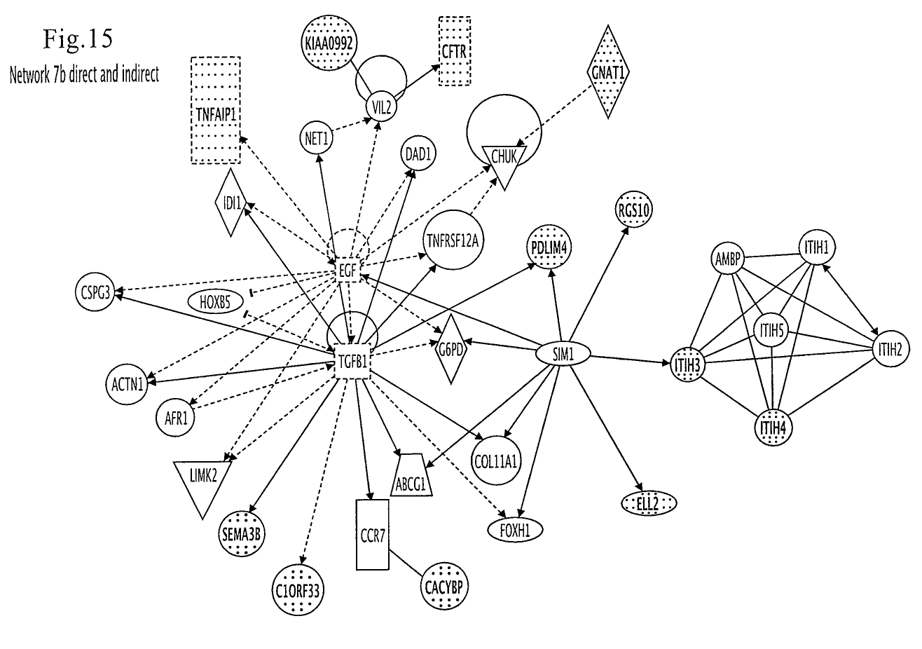

Network 6b contains 35 original nodes with 12 genes from the fine mapped regions (FIG. 14). The expression of 8 genes of this network has been shown to vary in some studies of gene expression profiling for CD. In the study by Langmann et al 2004, HGF and PAM3C have been shown to be upregulated in ilea of Crohn's patients compared to control specimens; TAGLN2 was downregulated in ileum; PCNA and ERCC5 were upregulated in colon; E4F1 and TKT were downregulated in colon; PTGS2 was upregulated in both colon and ileum.

For genes GDF9, MAC30, PAX7, POLDIP2, PPM1D, RAD50, RASSF1, RB1CC1, SALL1, TKT, UBE1L: please refer to descriptions included in the networks from direct analysis only.

USP19: ubiquitin specific peptidase 19. This gene encodes a deubiquitinating enzyme widely expressed in various tissues including skeletal muscle. Expression of this enzyme was increased in rat skeletal muscle during catabolic states (Combaret et al 2005).

Network 7b Direct and Indirect

Network 7 contains 35 original nodes with 12 genes from the fine mapped regions (FIG. 15). The expression of 5 genes of this network has been shown to vary in some studies of gene expression profiling for CD. In sigmoid colon tissue from Crohn's patients compared to control tissue, CHUK was upregulated (Costello et al 2005). In the study by Langmann et al 2004, CCR7 and IDI1 have been shown to be downregulated in ileum of Crohn's patient compared to control specimens; GLI2, CCNG1, ACTN1 and CFTR were upregulated in colon.

For genes C1orf33, CACYBP, CFTR, ELL2, GNAT1, ITIH3, ITIH4, KIAA0992, PDLIM4, RGS10, SEMA3B: please refer to descriptions included in the networks from direct analysis only.

TNFAIP1: TNFAIP1 is a novel tumor necrosis factor-alpha-induced endothelial primary response gene (Wolf et al., 1992). The response of endothelial cells to the cytokine tumor necrosis factor-alpha (TNF) is complex, involving the induction and suppression of multiple genes and gene products. TNF-alpha is an important cytokine mediator of immune regulation and inflammation, and can cause either beneficial or detrimental properties, through its proinflammatory and proapoptotic effects in various cell types.

Networks 8b to 17b Direct and Indirect

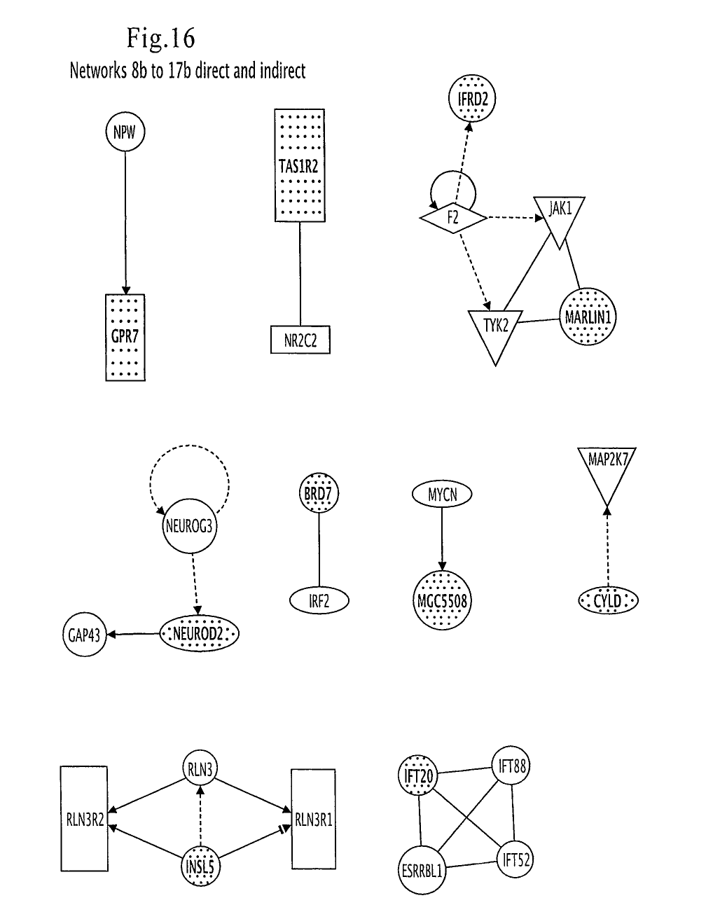

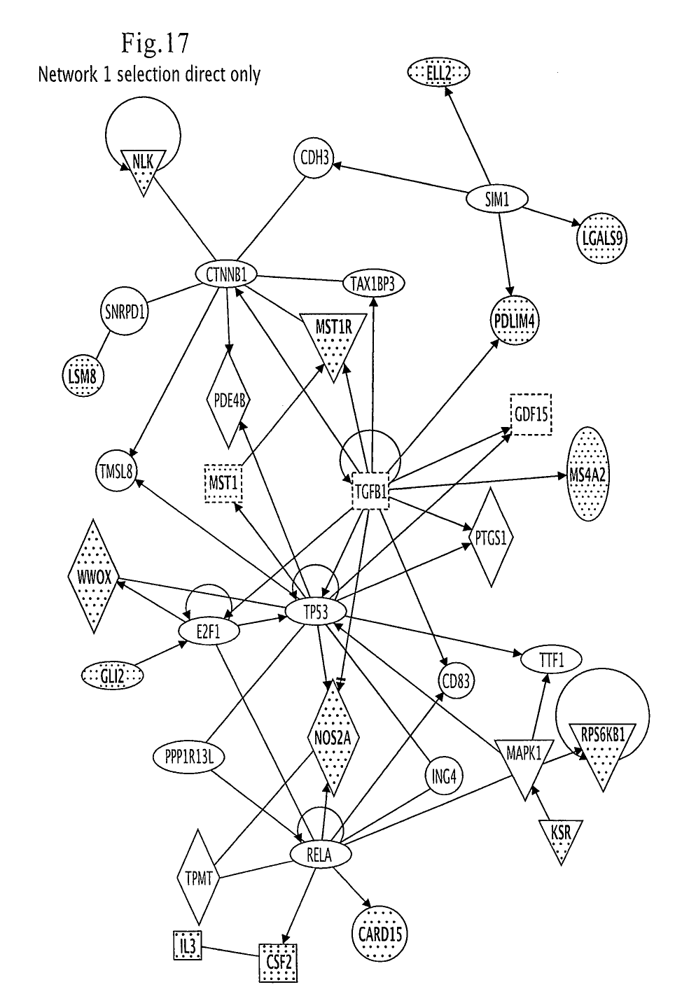

Networks 8b to 17b contain 26 original nodes with 10 genes from the fine mapped regions (FIG. 16). The expression of 5 genes of these networks has been shown to vary in some studies of gene expression profiling for CD. In sigmoid colon tissue from Crohn's patients compared to control tissue, CYLD was downregulated (Costello et al 2005). In the study by Langmann et al 2004, MYCN and ESRRBL1 have been shown to be upregulated in ileum of Crohn's patient compared to control specimens; IFRD2 was downregulated in ileum; MAP2K7 was downregulated in colon.

For genes BRD7, GPR7, IFT20, INSL5, MARLIN1, MGC5508, NEUROD2, TAS1 R2: please refer to descriptions included in the networks from direct analysis only.

CYLD: this gene encodes an antioncogene that may be involved in ubiquitin-dependent protein catabolism. Kolvalenko et al. (2003) have shown that CYLD negatively regulates NF-kappaB signaling by deubiquitination. They showed that CYLD interacts with NEMO, the regulatory subunit of IKK, and that it also interacts directly with tumour-necrosis factor receptor (TNFR)-associated factor 2 (TRAF2), an adaptor molecule involved in signaling by members of the family of TNF/nerve growth factor receptors. CYLD has also been shown to negatively regulate the c-Jun NH(2)-terminal kinase (JNK). The JNK-inhibitory function of CYLD appears to be specific for immune receptors because the CYLD knockdown has no significant effect on stress-induced JNK activation. Also, CYLD negatively regulates the activation of MAP2K7 (or MKK7), an upstream kinase known to mediate JNK activation by immune stimuli (Reiley et al 2004).

IFRD2: interferon-related developmental regulator 2. The function of this protein is unknown.

### Results from Analysis Incorporating both Direct and Indirect Interactions for 61 Annotated Genes

Sixty-one of the 295 genes were selected for analysis using the Ingenuity System based on their location with respect to the statistical evidence as well as potential involvement in the pathophysiology of Crohn's disease. FIGS. 17-23 contain network diagrams similar to those describe above, but are based on this subset of genes that are most likely to represent the susceptibility disease genes. Please refer to the gene descriptions above for information on these networks. FIG. 24 contains a description of the symbols used in the network diagrams.

Nucleic Acid Sequences

The nucleic acid sequences of the present invention may be derived from a variety of sources including DNA, cDNA, synthetic DNA, synthetic RNA, derivatives, mimetics or combinations thereof. Such sequences may comprise genomic DNA, which may or may not include naturally occurring introns, genic regions, nongenic regions, and regulatory regions. Moreover, such genomic DNA may be obtained in association with promoter regions or poly (A) sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means. The nucleic acids described herein are used in certain embodiments of the methods of the present invention for production of RNA, proteins or polypeptides, through incorporation into cells, tissues, or organisms. In one embodiment, DNA containing all or part of the coding sequence for the genes described in Tables 8, 9, 19, 20, 21, 22, 23 or 24, or the SNP markers described in Tables 2, 3, 4, 5, 6, 7 or 10, is incorporated into a vector for expression of the encoded polypeptide in suitable host cells. The invention also comprises the use of the nucleotide sequence of the nucleic acids of this invention to identify DNA probes for the genes described in Tables 8, 9, 19, 20, 21, 22, 23 or 24 or the SNP markers described in Tables 2, 3, 4, 5, 6, 7 or 10, PCR primers to amplify the genes described in Tables 8, 9, 19, 20, 21, 22, 23 or 24 or the SNP markers described in Tables 2, 3, 4, 5, 6, 7 or 10, nucleotide polymorphisms in the genes described in Tables 8, 9, 19, 20, 21, 22, 23 or 24, and regulatory elements of the genes described in Tables 8, 9, 19, 20, 21, 22, 23 or 24. The nucleic acids of the present invention find use as primers and templates for the recombinant production of Crohn's disease-associated peptides or polypeptides, for chromosome and gene mapping, to provide antisense sequences, for tissue distribution studies, to locate and obtain full length genes, to identify and obtain homologous sequences (wild-type and mutants), and in diagnostic applications.

Antisense Oligonucleotides

In a particular embodiment of the invention, an antisense nucleic acid or oligonucleotide is wholly or partially complementary to, and can hybridize with, a target nucleic acid (either DNA or RNA) having the sequence of SEQ ID NO:1, NO:3 or any SEQ ID from any Tables of the invention. For example, an antisense nucleic acid or oligonucleotide comprising 16 nucleotides can be sufficient to inhibit expression of at least one gene from Tables 8, 9, 19, 20, 21, 22, 23 or 24. Alternatively, an antisense nucleic acid or oligonucleotide can be complementary to 5′ or 3′ untranslated regions, or can overlap the translation initiation codon (5′ untranslated and translated regions) of at least one gene from Tables 8, 9, 19, 20, 21, 22, 23 or 24, or its functional equivalent. In another embodiment, the antisense nucleic acid is wholly or partially complementary to, and can hybridize with, a target nucleic acid that encodes a polypeptide from a gene described in Tables 8, 9, 19, 20, 21, 22, 23 or 24.

In addition, oligonucleotides can be constructed which will bind to duplex nucleic acid (i.e., DNA:DNA or DNA:RNA), to form a stable triple helix containing or triplex nucleic acid. Such triplex oligonucleotides can inhibit transcription and/or expression of a gene from Tables 8, 9, 19, 20, 21, 22, 23 or 24, or its functional equivalent (M. D. Frank-Kamenetskii et al., 1995). Triplex oligonucleotides are constructed using the basepairing rules of triple helix formation and the nucleotide sequence of the genes described in Tables 8, 9, 19, 20, 21, 22, 23 or 24.

The present invention encompasses methods of using oligonucleotides in antisense inhibition of the function of the genes from Tables 8, 9, 19, 20, 21, 22, 23 or 24. In the context of this invention, the term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention. Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some non-limiting examples of modifications at the 2′ position of sugar moieties which are useful in the present invention include OH, SH, SCH3, F, OCH3, OCN, O(CH2), NH2 and O(CH2)n CH3, where n is from 1 to about 10. Such oligonucleotides are functionally interchangeable with natural oligonucleotides or synthesized oligonucleotides, which have one or more differences from the natural structure. All such analogs are comprehended by this invention so long as they function effectively to hybridize with at least one gene from Tables 8, 9, 19, 20, 21, 22, 23 or 24 DNA or RNA to inhibit the function thereof.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As defined herein, a “subunit” is a base and sugar combination suitably bound to adjacent subunits through phosphodiester or other bonds. Antisense nucleic acids or oligonuicleotides can be produced by standard techniques (see, e.g., Shewmaker et al., U.S. Pat. No. 6,107,065). The oligonucleotides used in accordance with this invention may be conveniently and routinely made through the well-known technique of solid phase synthesis. Any other means for such synthesis may also be employed; however, the actual synthesis of the oligonucleotides is well within the abilities of the practitioner. It is also well known to prepare other oligonucleotides such as phosphorothioates and alkylated derivatives.

The oligonucleotides of this invention are designed to be hybridizable with RNA (e.g., mRNA) or DNA from genes described in Tables 8, 9, 19, 20, 21, 22, 23 or 24. For example, an oligonucleotide (e.g., DNA oligonucleotide) that hybridizes to mRNA from a gene described in Tables 8, 9, 19, 20, 21, 22, 23 or 24 can be used to target the mRNA for RnaseH digestion. Alternatively an oligonucleotide that can hybridize to the translation initiation site of the mRNA of a gene described in Tables 8, 9, 19, 20, 21, 22, 23 or 24 can be used to prevent translation of the mRNA. In another approach, oligonucleotides that bind to the double-stranded DNA of a gene from Tables 8, 9, 19, 20, 21, 22, 23 or 24 can be administered. Such oligonucleotides can form a triplex construct and inhibit the transcription of the DNA encoding polypeptides of the genes described in Tables 8, 9, 19, 20, 21, 22, 23 or 24. Triple helix pairing prevents the double helix from opening sufficiently to allow the binding of polymerases, transcription factors, or regulatory molecules. Recent therapeutic advances using triplex DNA have been described (see, e.g., J. E. Gee et al., 1994, Molecular and Immunologic Approaches, Futura Publishing Co., Mt. Kisco, N.Y.).

As non-limiting examples, antisense oligonucleotides may be targeted to hybridize to the following regions: mRNA cap region; translation initiation site; translational termination site; transcription initiation site; transcription termination site; polyadenylation signal; 3′ untranslated region; 5′ untranslated region; 5′ coding region; mid coding region; and 3′ coding region. Preferably, the complementary oligonucleotide is designed to hybridize to the most unique 5′ sequence of a gene described in Tables 8, 9, 19, 20, 21, 22, 23 or 24, including any of about 15-35 nucleotides spanning the 5′ coding sequence. In accordance with the present invention, the antisense oligonucleotide can be synthesized, formulated as a pharmaceutical composition, and administered to a subject. The synthesis and utilization of antisense and triplex oligonucleotides have been previously described (e.g., Simon et al., 1999; Barre et al., 2000; Elez et al., 2000; Sauter et al., 2000).

Alternatively, expression vectors derived from retroviruses, adenovirus, herpes or vaccinia viruses or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding a polypeptide from the genes described in Tables 8, 9, 19, 20, 21, 22, 23 or 24. These techniques are described both in Sambrook et al., 1989 and in Ausubel et al., 1992. For example, expression of at least one gene from Tables 8, 9, 19, 20, 21, 22, 23 or 24 can be inhibited by transforming a cell or tissue with an expression vector that expresses high levels of untranslatable sense or antisense sequences. Even in the absence of integration into the DNA, such vectors may continue to transcribe RNA molecules until they are disabled by endogenous nucleases. Transient expression may last for a month or more with a nonreplicating vector, and even longer if appropriate replication elements are included in the vector system. Various assays may be used to test the ability of gene-specific antisense oligonucleotides to inhibit the expression of at least one gene from Tables 8, 9, 19, 20, 21, 22, 23 or 24. For example, mRNA levels of the genes described in Tables 8, 9, 19, 20, 21, 22, 23 or 24 can be assessed by Northern blot analysis (Sambrook et al., 1989; Ausubel et al., 1992; J. C. Alwine et al. 1977; I. M. Bird, 1998), quantitative or semi-quantitative RT-PCR analysis (see, e.g., W. M. Freeman et al., 1999; Ren et al., 1998; J. M. Cale et al., 1998), or in situ hybridization (reviewed by A. K. Raap, 1998). Alternatively, antisense oligonucleotides may be assessed by measuring levels of the polypeptide from the genes described in Tables 8, 9, 19, 20, 21, 22, 23 or 24, e.g., by western blot analysis, indirect immunofluorescence and immunoprecipitation techniques (see, e.g., J. M. Walker, 1998, Protein Protocols on CD-ROM, Humana Press, Totowa, N.J.). Any other means for such detection may also be employed, and is well within the abilities of the practitioner.

Mapping Technologies

The present invention includes various methods which employ mapping technologies to map SNPs and polymorphisms. For purpose of clarity, this section comprises, but is not limited to, the description of mapping technologies that can be utilized to achieve the embodiments described herein. Mapping technologies may be based on amplification methods, restriction enzyme cleavage methods, hybridization methods, sequencing methods, and cleavage methods using agents.

Amplification methods include: self sustained sequence replication (Guatelli et al., 1990), transcriptional amplification system (Kwoh et al., 1989), Q-Beta Replicase (Lizardi et al., 1988), isothermal amplification (e.g. Dean et al., 2002; and Hefner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

Restriction enzyme cleavage methods include: isolating sample and control DNA, amplification (optional), digestion with one or more restriction endonucleases, determination of fragment length sizes by gel electrophoresis and comparing samples and controls. Differences in fragment length sizes between sample and control DNA indicates mutations in the sample DNA. Moreover, sequence specific ribozymes (see, e.g., U.S. Pat. No. 5,498,531) or DNAzyme (e.g. U.S. Pat. No. 5,807,718) can be used to score for the presence of specific mutations by development or loss of a ribozyme or DNAzyme cleavage site.

Hybridization methods include any measurement of the hybridization or gene expression levels, of sample nucleic acids to probes corresponding to about 2, 3, 4, 5, 6, 7, 8, 9, 10, 15, 20, 25, 30, 50, 75, 100, 200, 500, 1000 or more genes, or ranges of these numbers, such as about 2-10, about 10-20, about 20-50, about 50-100, about 100-200, about 200-500 or about 500-1000 genes of Table 2, 3, 4, 5, 6, 7, 8, 9, 10, 11, 12, 13, 14, 15, 16, 17 or 18.

SNPs and SNP maps of the invention can be identified or generated by hybridizing sample nucleic acids, e.g., DNA or RNA, to high density arrays or bead arrays containing oligonucleotide probes corresponding to the polymorphisms of Tables 2, 3, 4, 5, 6, 7 or 10 (see the Affymetrix arrays and Illumina bead sets at www.affymetrix.com and www.illumina.com and see Cronin et al., 1996; or Kozel et al., 1996).

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency, in this case in 6× SSPET at 37° C. (0.005% Triton X-100), to ensure hybridization and then subsequent washes are performed at higher stringency (e.g., 1× SSPET at 37° C.) to eliminate mismatched hybrid duplexes. Successive washes may be performed at increasingly higher stringency (e.g., down to as low as 0.25× SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes or gene fragments described in Table 2 refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes (see GeneChip® Expression Analysis Manual, Affymetrix, Rev. 3, which is herein incorporated by reference in its entirety).

The phrase “hybridizing specifically to” or “specifically hybridizes” refers to the binding, duplexing, or hybridizing of a molecule substantially to or only to a particular nucleotide sequence or sequences under stringent conditions when that sequence is present in a complex mixture (e.g., total cellular) DNA or RNA.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

A variety of sequencing reactions known in the art can be used to directly sequence nucleic acids for the presence or the absence of one or more polymorphisms of Tables 2, 3, 4, 5, 6, 7 or 10. Examples of sequencing reactions include those based on techniques developed by Maxam and Gilbert (1977) or Sanger (1977). It is also contemplated that any of a variety of automated sequencing procedures can be utilized, including sequencing by mass spectrometry (see, e.g. PCT International Publication No. WO 94/16101; Cohen et al., 1996; and Griffin et al., 1993), real-time pyrophosphate sequencing method (Ronaghi et al.,1998; and Permutt et al., 2001) and sequencing by hybridization (see e.g. Drmanac et al., 2002).

Other methods of detecting polymorphisms include methods in which protection from cleavage agents is used to detect mismatched bases in RNA/RNA, DNA/DNA or RNA/DNA heteroduplexes (Myers et al., 1985). In general, the technique of “mismatch cleavage” starts by providing heteroduplexes formed by hybridizing (labeled) RNA or DNA containing a wild-type sequence with potentially mutant RNA or DNA obtained from a sample. The double-stranded duplexes are treated with an agent who cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of a mutation or SNP (see, for example, Cotton et al., 1988; and Saleeba et al., 1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping polymorphisms. For example, the mutY enzyme of E. coli cleaves A at G/A mismatches (Hsu et al., 1994). Other examples include, but are not limited to, the MutHLS enzyme complex of E. coli (Smith and Modrich Proc. 1996) and Cel 1 from the celery (Kulinski et al., 2000) both cleave the DNA at various mismatches. According to an exemplary embodiment, a probe based on a polymorphic site corresponding to a polymorphism of Tables 2, 3, 4, 5, 6, 7 or 10 is hybridized to a cDNA or other DNA product from a test cell or cells. The duplex is treated with a DNA mismatch repair enzyme, and the cleavage products, if any, can be detected from electrophoresis protocols or the like. See, for example, U.S. Pat. No. 5,459,039. Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad et al., 2004).

In other embodiments, alterations in electrophoretic mobility can be used to identify polymorphisms in a sample. For example, single strand conformation polymorphism (SSCP) analysis can be used to detect differences in electrophoretic mobility between mutant and wild type nucleic acids (Orita et al., 1989; Cotton et al., 1993; and Hayashi 1992). Single-stranded DNA fragments of case and control nucleic acids will be denatured and allowed to renature. The secondary structure of single-stranded nucleic acids varies according to sequence. The resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et al., 1985). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoorn et al., 2000).

Examples of other techniques for detecting polymorphisms include, but are not limited to, selective oligonucleotide hybridization, selective amplification, selective primer extension, selective ligation, single-base extension, selective termination of extension or invasive cleavage assay. For example, oligonucleotide primers may be prepared in which the polymorphism is placed centrally and then hybridized to target DNA under conditions which permit hybridization only if a perfect match is found (Saiki et al., 1986; Saiki et al., 1989). Such oligonucleotides are hybridized to PCR amplified target DNA or a number of different mutations when the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

Hybridization assays may also be carried out with a temperature gradient following the principle of dynamic allele-specific hybridization or like e.g. Jobs et al., (2003); and Bourgeois and Labuda, (2004). For example, the hybridization is done using one of the two allele-specific oligonucleotides labeled with a fluorescent dye, and an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of an associated allele, a particular allele of a polymorphic locus, or the like (e.g. Jobs et al., 2003). Alternatively, the hybridization is done under a gradually decreasing temperature. In this case, both allele-specific oligonucleotides are hybridized to the template competitively. At high temperature none of the two probes are hybridized. Once the optimal temperature of the full-matched probe is reached, it hybridizes and leaves no target for the mismatched probe (e.g. Bourgeois and Labuda, 2004). In the latter case, if the allele-specific probes are differently labeled, then they are hybridized to a single PCR-amplified target. If the probes are labeled with the same dye, then the probe cocktail is hybridized twice to identical templates with only one labeled probe, different in the two cocktails, in the presence of the unlabeled competitive probe.

Alternatively, allele specific amplification technology that depends on selective PCR amplification may be used in conjunction with the present invention. Oligonucleotides used as primers for specific amplification may carry the associated allele, a particular allele of a polymorphic locus, or the like, also referred to as “mutation” of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the mutation to create cleavage-based detection (Gasparini et al., 1992). It is anticipated that in certain embodiments, amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., 1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNPs and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78 2003).

Another alternative includes the single-base extension/ligation assay using a molecular inversion probe, consisting of a single, long oligonucleotide (see e.g. Hardenbol et al., 2003). In such an embodiment, the oligonucleotide hybridizes on both side of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, and bead array or like. This reaction can be performed at a high multiplexed level.

In another alternative, the associated allele, a particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, and bead array or like. In the latter case, the extension oligonucleotide will contain a SNP-specific zip code tag.

In yet another alternative, a SNP is scored by selective termination of extension. The template is first amplified by PCR and the extension oligonucleotide hybridizes in the vicinity of the SNP locus, close to but not necessarily adjacent to it. The extension reaction is carried out using a thermostable polymerase such as ThermoSequenase (GE Healthcare) in the presence of a mix of dNTPs and at least one ddNTP. The latter has to terminate the extension at one of the allele of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, SNPs are detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Methods to Identify Agents that Modulate the Expression of a Nucleic Acid Encoding a Gene Involved in Crohn's Disease.

The present invention provides methods for identifying agents that modulate the expression of a nucleic acid encoding a gene from Tables 8, 9, 19, 20, 21, 22, 23 or 24. Such methods may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell. Such cells can be obtained from any parts of the body such as the GI track, colon, esophagus, stomach, rectum, jujenum, ileum, mucosa, submucosa, cecum, rectum, scalp, blood, dermis, epidermis, skin cells, cutaneous surfaces, intertrigious areas, genitalia, vessels and endothelium. Some non-limiting examples of cells that can be used are: muscle cells, nervous cells, blood and vessels cells, dermis, epidermis and other skin cells, T cell, mast cell, CD4+ lymphocyte, monocyte, macrophage, synovial cell, glial cell, villous intestinal cell, neutrophilic granulocyte, eosinophilic granulocyte, keratinocyte, lamina propria lymphocyte, intraepithelial lymphocyte, epithelial cells and lymphocytes.

In one assay format, the expression of a nucleic acid encoding a gene of the invention (see Tables 8, 9, 19, 20, 21, 22, 23 or 24) in a cell or tissue sample is monitored directly by hybridization to the nucleic acids of the invention. Cell lines or tissues are exposed to the agent to be tested under appropriate conditions and time and total RNA or mRNA is isolated by standard procedures such as those disclosed in Sambrook et al., (1989) Molecular Cloning: A Laboratory Manual, Cold Spring Harbor Laboratory Press).

Probes to detect differences in RNA expression levels between cells exposed to the agent and control cells may be prepared as described above. Hybridization conditions are modified using known methods, such as those described by Sambrook et al., and Ausubel et al., as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The chip or wafer can then be exposed to total cellular RNA or polyA RNA from a sample under conditions in which the affixed sequences will specifically hybridize to the RNA. By examining for the ability of a given probe to specifically hybridize to an RNA sample from an untreated cell population and from a cell population exposed to the agent, agents which up or down regulate expression are identified.

Methods to Identify Agents that Modulate the Activity of a Protein Encoded by a Gene Involved in Crohn's Disease.

The present invention provides methods for identifying agents that modulate at least one activity of the proteins described in Tables 8, 9, 19, 20, 21, 22, 23 or 24. Such methods may utilize any means of monitoring or detecting the desired activity. As used herein, an agent is said to modulate the expression of a protein of the invention if it is capable of up- or down-regulating expression of the protein in a cell. Such cells can be obtained from any parts of the body such as the GI track, colon, esophagus, stomach, rectum, jujenum, ileum, mucosa, submucosa, cecum, rectum, scalp, blood, dermis, epidermis, skin cells, cutaneous surfaces, intertrigious areas, genitalia, vessels and endothelium. Some non-limiting examples of cells that can be used are: muscle cells, nervous cells, blood and vessels cells, dermis, epidermis and other skin cells, T cell, mast cell, CD4+ lymphocyte, monocyte, macrophage, synovial cell, glial cell, villous intestinal cell, neutrophilic granulocyte, eosinophilic granulocyte, keratinocyte, lamina propria lymphocyte, intraepithelial lymphocyte, epithelial cells and lymphocytes.

In one format, the specific activity of a protein of the invention, normalized to a standard unit, may be assayed in a cell population that has been exposed to the agent to be tested and compared to an unexposed control cell population may be assayed. Cell lines or populations are exposed to the agent to be tested under appropriate conditions and times. Cellular lysates may be prepared from the exposed cell line or population and a control, unexposed cell line or population. The cellular lysates are then analyzed with the probe.

Antibody probes can be prepared by immunizing suitable mammalian hosts utilizing appropriate immunization protocols using the proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. (Rockford, Ill.) may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation. While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) or modifications which affect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid. The desired monoclonal antibodies may be recovered from the culture supernatant or from the ascites supernatant. Fragments of the monoclonal antibodies or the polyclonal antisera which contain the immunologically significant portion(s) can be used as antagonists, as well as the intact antibodies. Use of immunologically reactive fragments, such as Fab or Fab' fragments, is often preferable, especially in a therapeutic context, as these fragments are generally less immunogenic than the whole immunoglobulin. The antibodies or fragments may also be produced, using current technology, by recombinant means. Antibody regions that bind specifically to the desired regions of the protein can also be produced in the context of chimeras derived from multiple species. Antibody regions that bind specifically to the desired regions of the protein can also be produced in the context of chimeras from multiple species, for instance, humanized antibodies. The antibody can therefore be a humanized antibody or a human antibody, as described in U.S. Pat. No. 5,585,089 or Riechmann et al. (1988).

Agents that are assayed in the above method can be randomly selected or rationally selected or designed. As used herein, an agent is said to be randomly selected when the agent is chosen randomly without considering the specific sequences involved in the association of the protein of the invention alone or with its associated substrates, binding partners, etc. An example of randomly selected agents is the use of a chemical library or a peptide combinatorial library, or a growth broth of an organism. As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. The agents of the present invention can be, as examples, oligonucleotides, antisense polynucleotides, interfering RNA, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art.

In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention includes antibodies or fragments thereof that bind to a protein encoded by a gene in Tables 8, 9, 19, 20, 21, 22, 23 or 24. Antibody agents can be obtained by immunization of suitable mammalian subjects with peptides, containing as antigenic regions, those portions of the protein intended to be targeted by the antibodies (see section above of antibodies as probes for standard antibody preparation methodologies).

In yet another class of agents, the present invention includes peptide mimetics that mimic the three-dimensional structure of the protein encoded by a gene from Tables 8, 9, 19, 20, 21, 22, 23 or 24. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others. In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In another form, peptide analogs are commonly used in the pharmaceutical industry as non-peptide drugs with properties analogous to those of the template peptide. These types of non-peptide compounds are also referred to as peptide mimetics or peptidomimetics (Fauchere, 1986; Veber & Freidinger, 1985; Evans et al., 1987) which are usually developed with the aid of computerized molecular modeling. Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage using methods known in the art. Labeling of peptide mimetics usually involves covalent attachment of one or more labels, directly or through a spacer (e.g., an amide group), to non-interfering position(s) on the peptide mimetic that are predicted by quantitative structure-activity data and molecular modeling. Such non-interfering positions generally are positions that do not form direct contacts with the macromolecule(s) to which the peptide mimetic binds to produce the therapeutic effect. Derivitization (e.g., labeling) of peptide mimetics should not substantially interfere with the desired biological or pharmacological activity of the peptide mimetic. The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al., 1991) and the phage display method. The two hybrid method detects protein-protein interactions in yeast (Fields et al., 1989). The phage display method detects the interaction between an immobilized protein and a protein that is expressed on the surface of phages such as lambda and M13 (Amberg et al., 1993; Hogrefe et al., 1993). These methods allow positive and negative selection for protein-protein interactions and the identification of the sequences that determine these interactions.

Method to Diagnose Crohn's Disease

The present invention also relates to methods for diagnosing inflammatory bowel disease or a related disease, preferably Crohn's disease (CD), a disposition to such disease, predisposition to such a disease and/or disease progression. In some methods, the steps comprise contacting a target sample with (a) nucleic acid molecule(s) or fragments thereof and comparing the concentration of individual mRNA(s) with the concentration of the corresponding mRNA(s) from at least one healthy donor. An aberrant (increased or decreased) mRNA level of at least one gene from Tables 8, 9, 19, 20, 21, 22, 23 or 24, at least 5 or 10 genes from Tables 8, 9, 19, 20, 21, 22, 23 or 24, at least 50 genes from Tables 8, 9, 19, 20, 21, 22, 23 or 24, at least 100 genes from Tables 8, 9, 19, 20, 21, 22, 23 or 24 or at least 200 genes from Tables 8, 9, 19, 20, 21, 22, 23 or 24 determined in the sample in comparison to the control sample is an indication of Crohn's disease or a related disease or a disposition to such kinds of diseases. For diagnosis, samples are, preferably, obtained from inflamed colon tissue. Samples can also be obtained from any parts of the body such as the GI track, colon, esophagus, stomach, rectum, jujenum, ileum, mucosa, submucosa, cecum, rectum, scalp, blood, dermis, epidermis, skin cells, cutaneous surfaces, intertrigious areas, genitalia, vessels and endothelium. Some non-limiting examples of cells that can be used are: muscle cells, nervous cells, blood and vessels cells, dermis, epidermis and other skin cells, T cell, mast cell, CD4+ lymphocyte, monocyte, macrophage, synovial cell, glial cell, villous intestinal cell, neutrophilic granulocyte, eosinophilic granulocyte, keratinocyte, lamina propria lymphocyte, intraepithelial lymphocyte, epithelial cells and lymphocytes.

For analysis of gene expression, total RNA is obtained from cells according to standard procedures and, preferably, reverse-transcribed. Preferably, a DNAse treatment (in order to get rid of contaminating genomic DNA) is performed. Some non-limiting examples of cells that can be used are: muscle cells, nervous cells, blood and vessels cells, dermis, epidermis and other skin cells, T cell, mast cell, CD4+ lymphocyte, monocyte, macrophage, synovial cell, glial cell, villous intestinal cell, neutrophilic granulocyte, eosinophilic granulocyte, keratinocyte, lamina propria lymphocyte, intraepithelial lymphocyte, epithelial cells and lymphocytes.

The nucleic acid molecule or fragment is typically a nucleic acid probe for hybridization or a primer for PCR. The person skilled in the art is in a position to design suitable nucleic acids probes based on the information provided in the Tables of the present invention. The target cellular component, i.e. mRNA, e.g., in colon tissue, may be detected directly in situ, e.g. by in situ hybridization or it may be isolated from other cell components by common methods known to those skilled in the art before contacting with a probe. Detection methods include Northern blot analysis, RNase protection, in situ methods, e.g. in situ hybridization, in vitro amplification methods (PCR, LCR, QRNA replicase or RNA-transcription/amplification (TAS, 3SR), reverse dot blot disclosed in EP-B10237362) and other detection assays that are known to those skilled in the art. Products obtained by in vitro amplification can be detected according to established methods, e.g. by separating the products on agarose or polyacrylamide gels and by subsequent staining with ethidium bromide. Alternatively, the amplified products can be detected by using labeled primers for amplification or labeled dNTPs. Preferably, detection is based on a microarray.

The probes (or primers) (or, alternatively, the reverse-transcribed sample mRNAs) can be detectably labeled, for example, with a radioisotope, a bioluminescent compound, a chemiluminescent compound, a fluorescent compound, a metal chelate, or an enzyme.

The present invention also relates to the use of the nucleic acid molecules or fragments described above for the preparation of a diagnostic composition for the diagnosis of Crohn's disease or a disposition to such a disease.

The present invention also relates to the use of the nucleic acid molecules of the present invention for the isolation or development of a compound which is useful for therapy of Crohn's disease. For example, the nucleic acid molecules of the invention and the data obtained using said nucleic acid molecules for diagnosis of Crohn's disease might allow for the identification of further genes which are specifically dysregulated, and thus may be considered as potential targets for therapeutic interventions.

The invention further provides prognostic assays that can be used to identify subjects having or at risk of developing Crohn's disease. In such method, a test sample is obtained from a subject and the amount and/or concentration of the nucleic acid described in Tables 8, 9, 19, 20, 21, 22, 23 or 24 is determined; wherein the presence of an associated allele, a particular allele of a polymorphic locus, or the likes in the nucleic acids sequences of this invention (see SEQ ID from Tables 2,-10 and 12-18) can be diagnostic for a subject having or at risk of developing Crohn's. As used herein, a “test sample” refers to a biological sample obtained from a subject of interest. For example, a test sample can be a biological fluid, a cell sample, or tissue. A biological fluid can be, but is not limited to saliva, serum, mucus, urine, stools, spermatozoids, vaginal secretions, lymph, amiotic liquid, pleural liquid and tears. Cells can be, but are not limited to: muscle cells, nervous cells, blood and vessels cells, dermis, epidermis and other skin cells, T cell, mast cell, CD4+ lymphocyte, monocyte, macrophage, synovial cell, glial cell, villous intestinal cell, neutrophilic granulocyte, eosinophilic granulocyte, keranocyte, lamina propria lymphocyte, intraepithelial lymphocyte, epithelial cells and lymphocytes.

Furthermore, the prognostic assays described herein can be used to determine whether a subject can be administered an agent (e.g., an agonist, antagonist, peptidomimetic, polypeptide, nucleic acid such as antisense DNA or interfering RNA (RNAi), small molecule or other drug candidate) to treat Crohn's disease. Specifically, these assays can be used to predict whether an individual will have an efficacious response or will experience adverse events in response to such an agent. For example, such methods can be used to determine whether a subject can be effectively treated with an agent that modulates the expression and/or activity of a gene from Tables 8, 9, 19, 20, 21, 22, 23 or 24 or the nucleic acids described herein. In another example, an association study may be performed to identify polymorphisms from Tables 2, 3, 4, 5, 6, 7 or 10 that are associated with a given response to the agent, e.g., an efficacious response or the likelihood of one or more adverse events. Thus, one embodiment of the present invention provides methods for determining whether a subject can be effectively treated with an agent for a disease associated with aberrant expression or activity of a gene from Tables 8, 9, 19, 20, 21, 22, 23 or 24 in which a test sample is obtained and nucleic acids or polypeptides from Tables 8, 9, 19, 20, 21, 22, 23 or 24 are detected (e.g., wherein the presence of a particular level of expression of a gene from Tables 8, 9, 19, 20, 21, 22, 23 or 24 or a particular allelic variant of such gene, such as polymorphisms from Tables 2, 3, 4, 5, 6, 7 or 10 is diagnostic for a subject that can be administered an agent to treat a disorder such as Crohn's disease). In one embodiment, the method includes obtaining a sample from a subject suspected of having Crohn's disease or an affected individual and exposing such sample to an agent. The expression and/or activity of the nucleic acids and/or genes of the invention are monitored before and after treatment with such agent to assess the effect of such agent. After analysis of the expression values, one skilled in the art can determine whether such agent can effectively treat such subject. In another embodiment, the method includes obtaining a sample from a subject having or susceptible to developing Crohn's disease and determining the allelic constitution of polymorphisms from Tables 2, 3, 4, 5, 6, 7 or 10 that are associated with a particular response to an agent. After analysis of the allelic constitution of the individual at the associated polymorphisms, one skilled in the art can determine whether such agent can effectively treat such subject.

The methods of the invention can also be used to detect genetic alterations in a gene from Tables 8, 9, 19, 20, 21, 22, 23 or 24, thereby determining if a subject with the lesioned gene is at risk for a disease associated with Crohn's disease. In preferred embodiments, the methods include detecting, in a sample of cells from the subject, the presence or absence of a genetic alteration characterized by at least one alteration linked to or affecting the integrity of a gene from Tables 8, 9, 19, 20, 21, 22, 23 or 24 encoding a polypeptide or the misexpression of such gene. For example, such genetic alterations can be detected by ascertaining the existence of at least one of: (1) a deletion of one or more nucleotides from a gene from Tables 8, 9, 19, 20, 21, 22, 23 or 24; (2) an addition of one or more nucleotides to a gene from Tables 8, 9, 19, 20, 21, 22, 23 or 24; (3) a substitution of one or more nucleotides of a gene from Tables 8, 9, 19, 20, 21, 22, 23 or 24; (4) a chromosomal rearrangement of a gene from Tables 8, 9, 19, 20, 21, 22, 23 or 24; (5) an alteration in the level of a messenger RNA transcript of a gene from Tables 8, 9, 19, 20, 21, 22, 23 or 24; (6) aberrant modification of a gene from Tables 8, 9, 19, 20, 21, 22, 23 or 24, such as of the methylation pattern of the genomic DNA, (7) the presence of a non-wild type splicing pattern of a messenger RNA transcript of a gene from Tables 8, 9, 19, 20, 21, 22, 23 or 24; (8) inappropriate post-translational modification of a polypeptide encoded by a gene from Tables 8, 9, 19, 20, 21, 22, 23 or 24; and (9) alternative promoter use.

As described herein, there are a large number of assay techniques known in the art which can be used for detecting alterations in a gene from Tables 8, 9, 19, 20, 21, 22, 23 or 24. A preferred biological sample is a peripheral blood sample obtained by conventional means from a subject. Another preferred biological sample is a buccal swab. Other biological samples can be, but are not limited to, urine, stools, spermatozoids, vaginal secretions, lymph, amiotic liquid, pleural liquid and tears.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or alternatively, in a ligation chain reaction (LCR) (see, e.g., Landegran et al., 1988; and Nakazawa et al., 1994), the latter of which can be particularly useful for detecting point mutations in a gene from Tables 8, 9, 19, 20, 21, 22, 23 or 24 (see Abavaya et al., 1995). This method can include the steps of collecting a sample of cells from a patient, isolating nucleic acid (e.g., genomic DNA, mRNA, or both) from the cells of the sample, contacting the nucleic acid sample with one or more primers which specifically hybridize to a gene from Tables 8, 9, 19, 20, 21, 22, 23 or 24 under conditions such that hybridization and amplification of the nucleic acid from Tables 8, 9, 19, 20, 21, 22, 23 or 24 (if present) occurs, and detecting the presence or absence of an amplification product, or detecting the size of the amplification product and comparing the length to a control sample. PCR and/or LCR may be desirable to use as a preliminary amplification step in conjunction with some of the techniques used for detecting a mutation, an associated allele, a particular allele of a polymorphic locus, or the like described herein.

Alternative amplification methods include: self sustained sequence replication (Guatelli et al., 1990), transcriptional amplification system (Kwoh et al., 1989), Q-Beta Replicase (Lizardi et al., 1988), isothermal amplification (e.g. Dean et al., 2002); and Hafner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

In an alternative embodiment, alterations in a gene from Tables 8, 9, 19, 20, 21, 22, 23 or 24, from a sample cell can be identified by identifying changes in a restriction enzyme cleavage pattern. For example, sample and control DNA is isolated, amplified (optionally), digested with one or more restriction endonucleases, and fragment length sizes are determined by gel electrophoresis and compared. Differences in fragment length sizes between sample and control DNA indicate a mutation(s), an associated allele, a particular allele of a polymorphic locus, or the like in the sample DNA. Moreover, sequence specific ribozymes (see, e.g., U.S. Pat. No. 5,498,531 or DNAzyme e.g. U.S. Pat. No. 5,807,718) can be used to score for the presence of specific associated allele, a particular allele of a polymorphic locus, or the likes by development or loss of a ribozyme or DNAzyme cleavage site.

The present invention also relates to further methods for diagnosing Crohn's disease or a related disorder, preferably IBD, a disposition to such disorder, predisposition to such a disorder and/or disorder progression. In some methods, the steps comprise contacting a target sample with (a) nucleic molecule(s) or fragments thereof and determining the presence or absence of a particular allele of a polymorphism that confers a disorder-related phenotype (e.g., predisposition to such a disorder and/or disorder progression). The presence of at least one allele from Tables 2, 3, 4, 5, 6, 7 or 10 that is associated with Crohn's disease (“associated allele”), at least 5 or 10 associated alleles from Table Tables 2, 3, 4, 5, 6, 7 or 10, at least 50 associated alleles from Table Tables 2, 3, 4, 5, 6, 7 or 10 at least 100 associated alleles from Table Tables 2, 3, 4, 5, 6, 7 or 10, or at least 200 associated alleles from Table Tables 2, 3, 4, 5, 6, 7 or 10 determined in the sample is an indication of Crohn's disease or a related disorder, a disposition or predisposition to such kinds of disorders, or a prognosis for such disorder progression. Samples may be obtained from any parts of the body such as the GI track, colon, esophagus, stomach, rectum, jujenum, ileum, mucosa, submucosa, cecum, rectum, scalp, blood, dermis, epidermis, skin cells, cutaneous surfaces, intertrigious areas, genitalia, vessels and endothelium. Some non-limiting examples of cells that can be used are: muscle cells, nervous cells, blood and vessels cells, dermis, epidermis and other skin cells, T cell, mast cell, CD4+ lymphocyte, monocyte, macrophage, synovial cell, glial cell, villous intestinal cell, neutrophilic granulocyte, eosinophilic granulocyte, keratinocyte, lamina propria lymphocyte, intraepithelial lymphocyte, epithelial cells and lymphocytes.

In other embodiments, alterations in a gene from Tables 8, 9, 19, 20, 21, 22, 23 or 24 can be identified by hybridizing sample and control nucleic acids, e.g., DNA or RNA, to high density arrays or bead arrays containing tens to thousands of oligonucleotide probes (Cronin et al., 1996; Kozal et al., 1996). For example, alterations in a gene from Tables 8, 9, 19, 20, 21, 22, 23 or 24 can be identified in two dimensional arrays containing light-generated DNA probes as described in Cronin et al., (1996). Briefly, a first hybridization array of probes can be used to scan through long stretches of DNA in a sample and control to identify base changes between the sequences by making linear arrays of sequential overlapping probes. This step allows the identification of point mutations, associated alleles, particular alleles of a polymorphic locus, or the like. This step is followed by a second hybridization array that allows the characterization of specific mutations by using smaller, specialized probe arrays complementary to all variants, mutations, alleles detected. Each mutation array is composed of parallel probe sets, one complementary to the wild-type gene and the other complementary to the mutant gene.

In yet another embodiment, any of a variety of sequencing reactions known in the art can be used to directly sequence a gene from Tables 8, 9, 19, 20, 21, 22, 23 or 24 and detect an associated allele, a particular allele of a polymorphic locus, or the like by comparing the sequence of the sample gene from Tables 8, 9, 19, 20, 21, 22, 23 or 24 with the corresponding wild-type (control) sequence. Examples of sequencing reactions include those based on techniques developed by Maxam and Gilbert (1977) or Sanger (1977). It is also contemplated that any of a variety of automated sequencing procedures can be utilized when performing the diagnostic assays (Bio/Techniques 19:448, 1995) including sequencing by mass spectrometry (see, e.g. PCT International Publication No. WO 94/16101; Cohen et al., 1996; and Griffin et al. 1993), real-time pyrophosphate sequencing method (Ronaghi et al., 1998; and Permutt et al., 2001) and sequencing by hybridization (see e.g. Drmanac et al., 2002).

Other methods of detecting an associated allele, a particular allele of a polymorphic locus, or the likes in a gene from Tables 8, 9, 19, 20, 21, 22, 23 or 24 include methods in which protection from cleavage agents is used to detect mismatched bases in RNA/RNA, DNA/DNA or RNA/DNA heteroduplexes (Myers et al., 1985). In general, the technique of “mismatch cleavage” starts by providing heteroduplexes formed by hybridizing (labeled) RNA or DNA containing the wild-type gene sequence from Tables 8, 9, 19, 20, 21, 22, 23 or 24 with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of an associated allele, a particular allele of a polymorphic locus, or the like (see, for example, Cotton et al., 1988; Saleeba et al., 1992). In a preferred embodiment, the control DNA or RNA can be labeled for detection, as described herein.

In still another embodiment, the mismatch cleavage reaction employs one or more proteins that recognize mismatched base pairs in double-stranded DNA (so called “DNA mismatch repair” enzymes) in defined systems for detecting and mapping point an associated allele, a particular allele of a polymorphic locus, or the likes in a gene from Tables 8, 9, 19, 20, 21, 22, 23 or 24 cDNAs obtained from samples of cells. For example, the mutY enzyme of E. coli cleaves A at G/A mismatches (Hsu et al., 1994). Other examples include, but are not limited to, the MutHLS enzyme complex of E. coli (Smith and Modrich., 1996) and Cel 1 from the celery (Kulinski et al., 2000) both cleave the DNA at various mismatches. According to an exemplary embodiment, a probe based on a gene sequence from Tables 8, 9, 19, 20, 21, 22, 23 or 24 is hybridized to a cDNA or other DNA product from a test cell or cells. The duplex is treated with a DNA mismatch repair enzyme, and the cleavage products, if any, can be detected using electrophoresis protocols or the like. See, for example, U.S. Pat. No. 5,459,039. Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad et al., 2004).

In other embodiments, alterations in electrophoretic mobility can be used to identify an associated allele, a particular allele of a polymorphic locus, or the likes in genes from Tables 8, 9, 19, 20, 21, 22, 23 or 24. For example, single strand conformation polymorphism (SSCP) analysis can be used to detect differences in electrophoretic mobility between mutant and wild type nucleic acids (Orita et al., 1993; see also Cotton, 1993; and Hayashi et al., 1992). Single-stranded DNA fragments of sample and control nucleic acids from Tables 8, 9, 19, 20, 21, 22, 23 or 24 will be denatured and allowed to renature. The secondary structure of single-stranded nucleic acids varies according to sequence; the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et al., 1985). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoorn et al., 2000).

Examples of other techniques for detecting point mutations, an associated allele, a particular allele of a polymorphic locus, or the like include, but are not limited to, selective oligonucleotide hybridization, selective amplification, selective primer extension, selective ligation, single-base extension, selective termination of extension or invasive cleavage assay. For example, oligonucleotide primers may be prepared in which the known associated allele, particular allele of a polymorphic locus, or the like is placed centrally and then hybridized to target DNA under conditions which permit hybridization only if a perfect match is found (Saiki et al., 1986; Saiki et al., 1989). Such allele specific oligonucleotides are hybridized to PCR amplified target DNA of a number of different associated alleles, a particular allele of a polymorphic locus, or the likes where the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like locus is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

The hybridization may also be carried out with a temperature gradient following the principle of dynamic allele-specific hybridization or like (e.g. Jobs et al., 2003; and Bourgeois and Labuda, 2004). For example, the hybridization is done using one of the two allele-specific oligonucleotides labeled with a fluorescent dye, an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of the associated allele, particular allele of a polymorphic locus, or the like (e.g. Jobs et al. supra). Alternatively, the hybridization is done under a gradually decreasing temperature. In this case, both allele-specific oligonucleotides are hybridized to the template competitively. At high temperature none of the two probes is hybridized. Once the optimal temperature of the full-matched probe is reached, it hybridizes and leaves no target for the mismatched probe. In the latter case, if the allele-specific probes are differently labeled, then they are hybridized to a single PCR-amplified target. If the probes are labeled with the same dye, then the probe cocktail is hybridized twice to identical templates with only one labeled probe, different in the two cocktails, in the presence of the unlabeled competitive probe.

Alternatively, allele specific amplification technology that depends on selective PCR amplification may be used in conjunction with the present invention. Oligonucleotides used as primers for specific amplification may carry the associated allele, particular allele of a polymorphic locus, or the like of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the associated allele, particular allele of a polymorphic locus, or the like to create cleavage-based detection (Gasparini et al., 1992). It is anticipated that in certain embodiments amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., 1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNP and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78, 2003).

Another alternative includes the single-base extension/ligation assay using a molecular inversion probe, consisting of a single, long oligonucleotide (see e.g. Hardenbol et al., 2003). In such an embodiment, the oligonucleotide hybridizes on both sides of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, bead array or the like. This reaction can be performed at a highly multiplexed level.

In another alternative, the associated allele, particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, bead array or the like. In the latter case, the extension oligonucleotide will contain a SNP-specific zip code tag.

In yet another alternative, the variant is scored by selective termination of extension. The template is first amplified by PCR and the extension oligonucleotide hybridizes in vicinity to the SNP locus, close to but not necessarily adjacent to it. The extension reaction is carried out using a thermostable polymerase such as ThermoSequenase (GE Healthcare) in the presence of a mix of dNTPs and at least one ddNTP. The latter has to terminate the extension at one of the alleles of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, the associated allele, particular allele of a polymorphic locus, or the like is detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Other types of markers can also be used for diagnostic purposes. For example, microsatellites can also be useful to detect the genetic predisposition of an individual to a given disorder. Microsatellites consist of short sequence motifs of one or a few nucleotides repeated in tandem. The most common motifs are polynucleotide runs, dinucleotide repeats (particularly the CA repeats) and trinucleotide repeats. However, other types of repeats can also be used. The microsatellites are very useful for genetic mapping because they are highly polymorphic in their length. Microsatellite markers can be typed by various means, including but not limited to DNA fragment sizing, oligonucleotide ligation assay and mass spectrometry. For example, the locus of the microsatellite is amplified by PCR and the size of the PCR fragment will be directly correlated to the length of the microsatellite repeat. The size of the PCR fragment can be detected by regular means of gel electrophoresis. The fragment can be labeled internally during PCR or by using end-labeled oligonucleotides in the PCR reaction (e.g. Mansfield et al., 1996). Alternatively, the size of the PCR fragment is determined by mass spectrometry. In such a case, however, the flanking sequences need to be eliminated. This can be achieved by ribozyme cleavage of an RNA transcript of the microsatellite repeat (Krebs et al., 2001). For example, the microsatellite locus is amplified using oligonucleotides that include a T7 promoter on one end and a ribozyme motif on the other end. Transcription of the amplified fragments will yield an RNA substrate for the ribozyme, releasing small RNA fragments that contain the repeated region. The size of the latter is determined by mass spectrometry. Alternatively, the flanking sequences are specifically degraded. This is achieved by replacing the dTTP in the PCR reaction by dUTP. The dUTP nucleosides are then removed by uracyl DNA glycosylases and the resulting abasic sites are cleaved by either abasic endonucleases such as human AP endonuclease or chemical agents such as piperidine. Bases can also be modified post-PCR by chemical agents such as dimethyl sulfate and then cleaved by other chemical agents such as piperidine (see e.g. Maxam and Gilbert, 1977; U.S. Pat. No. 5,869,242; and U.S. Patent pending Ser. No. 60/335,068).

In another alternative, an oligonucleotide ligation assay can be performed. The microsatellite locus is first amplified by PCR. Then, different oligonucleotides can be submitted to ligation at the center of the repeat with a set of oligonucleotides covering all the possible lengths of the marker at a given locus (Zirvi et al., 1999). Another example of design of an oligonucleotide assay comprises the ligation of three oligonucleotides; a 5′ oligonucleotide hybridizing to the 5′ flanking sequence, a repeat oligonucleotide of the length of the shortest allele of the marker hybridizing to the repeated region and a set of 3′ oligonucleotides covering all the existing alleles hybridizing to the 3′ flanking sequence and a portion of the repeated region for all the alleles longer than the shortest one. For the shortest allele, the 3′ oligonucleotide exclusively hybridizes to the 3′ flanking sequence (U.S. Pat. No. 6,479,244).

The methods described herein may be performed, for example, by utilizing pre-packaged diagnostic kits comprising at least one probe nucleic acid selected from the SEQ ID of Tables 2, 3, 4, 5, 6, 7 or 10, or antibody reagent described herein, which may be conveniently used, for example, in a clinical setting to diagnose patient exhibiting symptoms or a family history of a disorder or disorder involving abnormal activity of genes from Tables 8, 9, 19, 20, 21, 22, 23 or 24.

Method to Treat an Animal Suspected of Having Crohn's Disease

The present invention provides methods of treating a disease associated with Crohn's disease by expressing in vivo the nucleic acids of at least one gene from Tables 8, 9, 19, 20, 21, 22, 23 or 24. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids encoding a gene from Tables 8, 9, 19, 20, 21, 22, 23 or 24, under the control of a promoter, then express the encoded protein, thereby mitigating the effects of absent, partial inactivation, or abnormal expression of a gene from Tables 8, 9, 19, 20, 21, 22, 23 or 24.

Such gene therapy procedures have been used to correct acquired and inherited genetic defects, cancer, and viral infection in a number of contexts. The ability to express artificial genes in humans facilitates the prevention and/or cure of many important human disorders, including many disorders which are not amenable to treatment by other therapies (for a review of gene therapy procedures, see Anderson, 1992; Nabel & Feigner, 1993; Mitani & Caskey, 1993; Mulligan, 1993; Dillon, 1993; Miller, 1992; Van Brunt, 1998; Vigne, 1995; Kremer & Perricaudet 1995; Doerfler & Bohm 1995; and Yu et al., 1994).

Delivery of the gene or genetic material into the cell is the first critical step in gene therapy treatment of a disorder. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see the references included in the above section.

The use of RNA or DNA based viral systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., 1992; Johann et al., 1992; Sommerfelt et al., 1990; Wilson et al., 1989; Miller et al., 1999; and PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., 1987; U.S. Pat. No. 4,797,368; WO 93/24641; Kotin, 1994; Muzyczka, 1994). Construction of recombinant AAV vectors is described in a number of publications, including U.S. Pat. No. 5,173,414; Tratschin et al., 1985; Tratschin, et al., 1984; Hermonat & Muzyczka, 1984; and Samulski et al., 1989.

In particular, numerous viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors (Ellem at al., 1997; and Dranoff et al., 1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 by inverted terminal repeats flanking the transgene expression cassette. Efficient gene transfer and stable transgene delivery due to integration into the genomes of the transduced cell are key features for this vector system (Wagner et al., 1998, Kearns et al., 1996).

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply the deleted gene function in trans. Ad vectors can transduce multiple types of tissues in vivo, including nondividing, differentiated cells such as those found in the liver, kidney and muscle tissues. Conventional Ad vectors have a large carrying capacity. An example of the use of an Ad vector in a clinical trial involved polynucleotide therapy for antitumor immunization with intramuscular injection (Sterman et al., 1998). Additional examples of the use of adenovirus vectors for gene transfer in clinical trials include Rosenecker et al., 1996; Sterman et al., 1998; Welsh et al., 1995; Alvarez et al., 1997; Topf et al., 1998.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, reported that Moloney murine leukemia virus can be modified to express human heregulin fused to gp70, and the recombinant virus infects certain human breast cancer cells expressing human epidermal growth factor receptor. This principle can be extended to other pairs of viruses expressing a ligand fusion protein and target cells expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, and tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., 1994; and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells).

Vectors (e.g., retroviruses, adenoviruses, liposomes, etc.) containing therapeutic nucleic acids can be also administered directly to the organism for transduction of cells in vivo. Alternatively, naked DNA can be administered.

Administration is by any of the routes normally used for introducing a molecule into ultimate contact with blood or tissue cells, as described above. The nucleic acids from Tables 8, 9, 19, 20, 21, 22, 23 or 24 are administered in any suitable manner, preferably with the pharmaceutically acceptable carriers described above. Suitable methods of administering such nucleic acids are available and well known to those of skill in the art, and, although more than one route can be used to administer a particular composition, a particular route can often provide a more immediate and more effective reaction than another route (see Samulski et al., 1989). The present invention is not limited to any method of administering such nucleic acids, but preferentially uses the methods described herein.

The present invention further provides other methods of treating Crohn's disease such as administering to an individual having Crohn's disease an effective amount of an agent that regulates the expression, activity or physical state of at least one gene from Tables 8, 9, 19, 20, 21, 22, 23 or 24. An “effective amount” of an agent is an amount that modulates a level of expression or activity of a gene from Tables 8, 9, 19, 20, 21, 22, 23 or 24, in a cell in the individual at least about 10%, at least about 20%, at least about 30%, at least about 40%, at least about 50%, at least about 60%, at least about 70%, at least about 80% or more, compared to a level of the respective gene from Tables 8, 9, 19, 20, 21, 22, 23 or 24 in a cell in the individual in the absence of the compound. The preventive or therapeutic agents of the present invention may be administered, either orally or parenterally, systemically or locally. For example, intravenous injection such as drip infusion, intramuscular injection, intraperitoneal injection, subcutaneous injection, suppositories, intestinal lavage, oral enteric coated tablets, and the like can be selected, and the method of administration may be chosen, as appropriate, depending on the age and the conditions of the patient. The effective dosage is chosen from the range of 0.01 mg to 100 mg per kg of body weight per administration. Alternatively, the dosage in the range of 1 to 1000 mg, preferably 5 to 50 mg per patient may be chosen. The therapeutic efficacy of the treatment may be monitored by observing various parts of the GI tract, by endoscopy, barium, colonoscopy, or any other monitoring methods known in the art. Other ways of monitoring efficacy can be, but are not limited to monitoring inflammatory conditions involving the upper gastrointestinal tract such as monitoring the amelioration on the esophageal discomfort, decrease in pain, improved swallowing, reduced chest pain, decreased heartburn, decreased regurgitation of solids or liquids after swallowing or eating, decrease in vomiting, or improvement in weight gain or improvement in vitality.

The present invention further provides a method of treating an individual clinically diagnosed with Crohns' disease. The methods generally comprises analyzing a biological sample that includes a cell, in some cases, a GI track cell, from an individual clinically diagnosed with Crohn's disease for the presence of modified levels of expression of at least 1 gene, at least 10 genes, at least 50 genes, at least 100 genes, or at least 200 genes from Tables 8, 9, 19, 20, 21, 22, 23 or 24. A treatment plan that is most effective for individuals clinically diagnosed as having a condition associated with Crohn's disease is then selected on the basis of the detected expression of such genes in a cell. Treatment may include administering a composition that includes an agent that modulates the expression or activity of a protein from Tables 8, 9, 19, 20, 21, 22, 23 or 24 in the cell. Information obtained as described in the methods above can also be used to predict the response of the individual to a particular agent. Thus, the invention further provides a method for predicting a patient's likelihood to respond to a drug treatment for a condition associated with Crohn's disease, comprising determining whether modified levels of a gene from Tables 8, 9, 19, 20, 21, 22, 23 or 24 is present in a cell, wherein the presence of protein is predictive of the patient's likelihood to respond to a drug treatment for the condition. Examples of the prevention or improvement of symptoms accompanied by Crohn's disease that can monitored for effectiveness include prevention or improvement of diarrhea, prevention or improvement of weight loss, inhibition of bowel tissue edema, inhibition of cell infiltration, inhibition of surviving period shortening, and the like, and as a result, a preventing or improving agent for diarrhea, a preventing or improving agent for weight loss, an inhibitor for bowel tissues edema, an inhibitor for cell infiltration, an inhibitor for surviving period shortening, and the like can be identified.

The invention also provides a method of predicting a response to therapy in a subject having Crohn's disease by determining the presence or absence in the subject of one or more markers associated with Crohn's disease described in Tables 2, 3, 4, 5, 6, 7 or 10, diagnosing the subject in which the one or more markers are present as having Crohn's disease, and predicting a response to a therapy based on the diagnosis e.g., response to therapy may include an efficacious response and/or one or more adverse events. The invention also provides a method of optimizing therapy in a subject having Crohn's disease by determining the presence or absence in the subject of one or more markers associated with a clinical subtype of Crohn's disease, diagnosing the subject in which the one or more markers are present as having a particular clinical subtype of Crohn's disease, and treating the subject having a particular clinical subtype of Crohn's disease based on the diagnosis. As an example, treatment for the fibrostenotic subtype of Crohn's disease currently includes surgical removal of the affected, strictured part of the bowel.

Thus, while there are a number of treatments for Crohn's disease currently available, they all are accompanied by various side effects, high costs, and long complicated treatment protocols, which are often not available and effective in a large number of individuals. Accordingly, there remains a need in the art for more effective and otherwise improved methods for treating and preventing Crohn's disease. Thus, there is a continuing need in the medical arts for genetic markers of Crohn's disease and guidance for the use of such markers. The present invention fulfills this need and provides further related advantages.

### EXAMPLES

### Example 1

### Identification of Cases and Controls

All individuals were sampled from the Quebec founder population (QFP). Membership in the founder population was defined as having four grandparents with French Canadian family names who were born in the Province of Quebec, Canada or in adjacent areas of the Provinces of New Brunswick and Ontario or in New England or New York State. The Quebec founder population has two distinct advantages over general populations for LD mapping. Because it is relatively young (about 12 to 15 generations from the mid 17th century to the present) and because it has a limited but sufficient number of founders (approximately 2600 effective founders, Charbonneau et al. 1987), the Quebec population is characterized both by extended LD and by decreased genetic heterogeneity. The increased extent of LD allows the detection of disease associated genes using a reasonable marker density, while still allowing the increased meiotic resolution of population-based mapping. The number of founders is small enough to result in increased LD and reduced allelic heterogeneity, yet large enough to insure that all of the major disease genes involved in general populations are present in Quebec. Reduced allelic heterogeneity will act to increase relative risk imparted by the remaining alleles and so increase the power of case/control studies to detect genes and gene alleles involved in complex disorders within the Quebec population. The specific combination of age in generations, optimal number of founders and large present population size makes the QFP optimal for LD-based gene mapping.

Patient inclusion criteria for the study include diagnosis for Crohn's disease by any one of the following: a colonoscopy, a radiological examination with barium, an abdominal surgical operation or a biopsy or a surgical specimen. The colonoscopy diagnosis consists of observing linear, deep or serpentigenous ulcers, pseudopolyps, or skip areas. The barium radiological examination consists of the detection of strictures, ulcerations and string signs by observing the barium enema and the small bowel followed through an NMRI series.

Patients that were diagnosed with ulcerative colitis, infectious colitis or other intestinal diseases were excluded from the study. All human sampling was subject to ethical review procedures.

All enrolled QFP subjects (patients and controls) provided a 30 ml blood sample (3 barcoded tubes of 10 ml). Samples were processed immediately upon arrival at Genizon's laboratory. All samples were scanned and logged into a LabVantage Laboratory Information Management System (LIMS), which served as a hub between the clinical data management system and the genetic analysis system. Following centrifugation, the buffy coat containing the white blood cells was isolated from each tube. Genomic DNA was extracted from the buffy coat from one of the tubes, and stored at 4° C. until required for genotyping. DNA extraction was performed with a commercial kit using a guanidine hydrochloride based method (FlexiGene, Qiagen) according to the manufacturer's instructions. The extraction method yielded high molecular weight DNA, and the quality of every DNA sample was verified by agarose gel electrophoresis. Genomic DNA appeared on the gel as a large band of very high molecular weight. The remaining two buffy coats were stored at −80° C. as backups.

The QFP samples were collected as family trios consisting of Crohn's disease subjects and two first degree relatives. Of the 500 trios, 477 were Parent, Parent, Child (PPC) trios; the remainders were Parent, Child, Child (PCC) trios. Only the PPC trios were used for the analysis reported here because they produced equal numbers of more accurately estimated case and control haplotypes than the PCC trios. 382 trios were used in the genome wide scan and 477 trios were used in the fine mapping component of the study. One member of each trio was affected with Crohn's disease. For the 382 trios used in the genome wide scan, these included 189 daughters, 90 sons, 54 mothers and 49 fathers. When a child was the affected member of the trio, the two non-transmitted parental chromosomes (one from each parent) were used as controls, when one of the parents was affected, that person's spouse provided the control chromosomes. The recruitment of trios allowed a more precise determination of long extended haplotypes.

In addition, samples from a European general population (German samples) were also recruited with similar exclusion and inclusion criteria described above for the QFP samples. Two sets of samples were collected: trios (PPC) and unrelated single samples (cases and controls). All individuals recruited were required to have parents of Caucasian origin born in Germany. Five hundred trios (2 parents and one child) and 750 cases and 750 controls were genotyped as described in the Fine Mapping example below (Example 4).

### Example 2

### Genome Wide Association

Genotyping was performed using Perlegen's ultra-high-throughput platform. Marker loci were amplified by PCR and hybridized to wafers containing arrays of oligonucleotides. Allele discrimination was performed through allele-specific hybridization. In total, 248,535 SNPs, distributed as evenly as possible throughout the genome, were genotyped on the 382 QFP trios for a total of 372,802,500 genotypes. These markers were mostly selected from various databases including the ˜1.6 million SNP database of Perlegen Life Sciences (Patil, 2001); several thousand were obtained from the HapMap consortium database and/or dbSNP at NCBI. The SNPs were chosen to maximize uniformity of genetic coverage and to cover a distribution of allele frequencies. All SNPs that did not pass the quality controls for the assay, that is, that had a minor allele frequency of less than 1%, a Mendelian error rate within trios greater than 1%, that deviated significantly from the Hardy-Weinberg equilibrium, or that had excessive missing data (cut-off at 5% missing values or higher) were removed from the analysis. Genetic analysis was performed on a total of 165,785 SNPs (158,775 autosomal, 6869 X chromosome and 141 Y chromosome). The average gap size was approximately 17 kb. Of the 165,785 markers, ˜140,000 had a minor allele frequency (MAF) greater than 10% for the QFP.

The genotyping information was entered into a Unified Genotype Database (a proprietary database under development) from which it was accessed using custom-built programs for export to the genetic analysis pipeline. Analyses of these genotypes were performed with the statistical tools described in Example 3. The GWS permitted the identification of 31 candidate chromosomal regions linked to Crohn's disease (Table 1). These regions were further analyzed by the Fine Mapping approach described in Example 4.

### Example 3

### Genetic Analysis

1. Dataset Quality Assessment

Prior to performing any analysis, the dataset from the GWS was verified for completeness of the trios. The program GGFileMod removed any trios with abnormal family structure or missing individuals (e.g. trios without a proband, duos, singletons, etc.), and calculated the total number of complete trios in the dataset. The trios were also tested to make sure that no subjects within the cohort were related more closely than second cousins (6 meiotic steps).

Subsequently, the program DataCheck2.1 was used to calculate the following statistics per marker and per family:
Minor allele frequency (MAF) for each marker; Missing values for each marker and family; Hardy Weinberg Equilibrium for each marker; and Mendelian segregation error rate.

The following acceptance criteria were applied for internal analysis purposes:
MAF>1%;
Missing values <1%;
Observed non-Mendelian segregation <0.33%;
Non significant deviation in allele frequencies from Hardy Weinberg equilibrium.

Markers or families not meeting these criteria were removed from the dataset in the following step. Analyses of variance were performed using the algorithm GenAnova, to assess whether families or markers have a greater effect on missing values and/or non-Mendelian segregation. This was used to determine the smallest number of data points to remove from the dataset in order to meet the requirements for missing values and non-Mendelian segregation. The families and/or markers were removed from the dataset using the program DataPull, which generates an output file that is used for subsequent analysis of the genotype data.

2. Phase Determination

The program PhaseFinderSNP2.0 was used to determine phase from trio data on a marker-by-marker, trio-by-trio basis. The output file contains haplotype data for all trio members, with ambiguities present when all trio members are heterozygous or where data is missing. The program FileWriterTemp was then used to determine case and control haplotypes and to prepare the data in the proper input format for the next stage of analysis, using the expectation maximization algorithm, PL-EM, to call phase on the remaining ambiguities. This stage consists of several modules for resolution of the remaining phase ambiguities. PLEMInOut1 was first used to recode the haplotypes for input into the PL-EM algorithm in 15-marker blocks for the genome wide scan data and for 11 marker blocks for fine and ultra-fine mapping data sets. The haplotype information was encoded as genotypes, allowing for the entry of known phase into the algorithm; this method limits the possible number of estimated haplotypes conditioned on already known phase assignments. The PL-EM algorithm was used to estimate haplotypes from the “pseudo-genotype” data in 11 or 15-marker windows, advancing in increments of one marker across the chromosome. The results were then converted into multiple haplotype files using the program PLEMInOut2. Subsequently PLEMBlockGroup was used to convert the individual 11 or 15-marker block files into one continuous block of haplotypes for the entire chromosome, and to generate files for further analysis by LDSTATS and SINGLETYPE. PLEMBlockGroup takes the consensus estimation of the allele call at each marker over all separate estimations (most markers are estimated 11-15 different times as the 11 or 15 marker blocks pass over their position).

3. Haplotype Association Analysis

Haplotype association analysis was performed using the program LDSTATS. LDSTATS tests for association of haplotypes with the disease phenotype. The algorithms LDSTATS (v2.0) and LDSTATS (v4.0) define haplotypes using multi-marker windows that advance across the marker map in one-marker increments. Windows can contain any odd number of markers specified as a parameter of the algorithm. Other marker windows can also be used. At each position the frequency of haplotypes in cases and controls was calculated and a chi-square statistic was calculated from case control frequency tables. For LDSTATS v2.0, the significance of the chi-square for single marker and 3-marker windows was calculated as Pearson's chi-square with degrees of freedom. Larger windows of multi-allelic haplotype association were tested using Smith's normalization of the square root of Pearson's Chi-square. In addition, LDSTATS v2.0 calculates Chi-square values for the transmission disequilibrium test (TDT) for single markers in situations where the trios consisted of parents and an affected child.

LDSTATS v4.0 calculates significance of chi-square values using a permutation test in which case-control status is randomly permuted until 350 permuted chi-square values are observed that are greater than or equal to chi-square value of the actual data. The P value is then calculated as 350/the number of permutations required.

Table 2 lists the results for association analysis using LDSTATs (v2.0 and v4.0) for the 31 regions described above based on the genome wide scan genotype data for 382 QFP trios. For each region that was associated with Crohn disease in the genome wide scan, we report in Table 13 the allele frequencies and the relative risk (RR) for the haplotypes contributing to the best signal at each SNP in the region. The best signal at a given location was determined by comparing the significance (p-value) of the association with Crohn disease for window sizes of 1, 3, 5, 7, and 9 SNPs, and selecting the most significant window. For a given window size at a given location, the association with Crohn disease was evaluated by comparing the overall distribution of haplotypes in the cases with the overall distribution of haplotypes in the controls. Haplotypes with a relative risk greater than one increase the risk of developing Crohn disease while haplotypes with a relative risk less than one are protective and decrease the risk.

4. Conditional Haplotype Analyses

Conditional haplotype analyses were performed on subsets of the original set of 382 cases and 382 controls using the program LDSTATS (v2.0). The selection of a subset of cases and their matched controls was based on the carrier status of cases at a gene or locus of interest. We selected the gene NOD2 (alias CARD15) on chromosome 16 and the gene IL23R on chromosome 1 based on our association findings using LDSTAT (v2.0) in our fine mapping of these two loci with 477 trios (see below). The most significant association signal in NOD2 was obtained with a haplotype window of size 7 containing SNPs corresponding to SEQ IDs 13789, 13796, 13799, 13800, 13802, 13804 and 13805 (see Table below for conversion to the specific DNA alleles used). A reduced haplotype diversity was observed and we selected two sets of risk haplotypes for conditional analyses. The first set consisted of haplotypes 2121222 and 1121211 and the second set contained the above two haplotypes and haplotype 2121211. Using the first set, we partitioned the cases into two groups; the first group consisting of those cases that were carrier of a risk haplotype and the second group consisting of the remaining cases, the non carriers. The resulting sample sizes were respectively 125 and 227. LDSTAT (v2.0) was run in each group and regions showing association with Crohn disease are reported in Table 11. Four regions (C02, M02, N01 and S03) were associated with Crohn disease in the group of non carriers (not_NOD2_caserisk_{—}2hap), indicating the existence of risk factors acting independently of NOD2. We repeated the process of partitioning the cases into two groups using the second set containing three risk haplotypes. The resulting sample sizes were 200 and 152 for the two groups of cases. One region (S03) was associated with Crohn disease in the group of non carriers (not_NOD2_caserisk_{—}3hap) indicating the presence of risk factors acting independently of NOD2 (Table 11).

A second conditional analysis was performed using the gene IL23R on chromosome 1. The most significant association in IL23R was obtained with a haplotype window of size 9 containing SNPs corresponding to SEQ IDs 5382, 5387, 5390, 5393, 5396, 5397, 5401, 5404 and 5406 (see Table below for conversion to the specific DNA alleles used). A reduced haplotype diversity was observed and we selected one set of protective haplotypes and a risk haplotype for conditional analyses. The protective set consisted of haplotypes 212111122 and 212111121 and the risk haplotype was 221211121. In addition, due to dominance effects involving the risk haplotype, we also considered the risk haplotype 221211121 while excluding heterozygotes involving haplotypes 122122212, 222122211 and 212111122 (the “exclusion” set) and the risk haplotype. Using the first set of protective haplotypes, we partitioned the cases into two groups; the first group consisting of those cases that were carrier of a protective haplotype and the second group consisting of the remaining cases, the non carriers. The resulting sample sizes were respectively 204 and 162. LDSTATS (v2.0) was run in each group and regions showing association with Crohn disease are reported in Table 11. One region (S02) was associated with Crohn disease in the group of carriers (has_IL23R_caseprotective_{—}2hap), indicating the existence of risk factors acting independently of IL23R. Four regions (C02, C03, M02, T04) were associated with Crohn disease in the group of non carriers (not_IL23R_caseprotective_{—}2hap), indicating the presence of an epistatic interaction between risk factors in those regions and risk factors in IL23R (Table 11). We repeated the process of partitioning the cases into two groups using the risk haplotype in IL23R. The resulting sample sizes were 205 and 161 for the group of carriers and non carriers respectively. Four regions (C02, N01, S02, V04) were associated with Crohn disease in the group of non carriers (not_IL23R_caserisk) indicating the presence of risk factors acting independently of IL23R (Table 11). In a similar fashion, we partitioned the cases into two groups using the risk haplotype and the exclusion set. The carriers were cases with the risk haplotype but without one of the haplotypes in the exclusion set. The non carriers were the remaining set of cases. The sample sizes for the two groups were 184 and 182 respectively. One region (S02) was associated with Crohn disease in the group of carriers (has_IL23R_caserisk_not3) indicating the presence of an epistatic interaction between risk factors in that region and risk factors in IL23R (Table 11). Four regions (T04, V03, X01, X03) were associated with Crohn disease in the group of non carriers (not_IL23R_caserisk_not3) indicating the presence of risk factors acting independently of IL23R (Table 11).

For each region that was associated with Crohn disease in the conditional analyses, we report in Table 12 the allele frequencies and the relative risk (RR) for the haplotypes contributing to the best signal at each SNP in the region. The best signal at a given location was determined by comparing the significance (p-value) of the association with Crohn disease for window sizes of 1, 3, 5, 7, and 9 SNPs, and selecting the most significant window. For a given window size at a given location, the association with Crohn disease was evaluated by comparing the overall distribution of haplotypes in the cases with the overall distribution of haplotypes in the controls. Haplotypes with a relative risk greater than one increase the risk of developing Crohn disease while haplotypes with a relative risk less than one are protective and decrease the risk.

DNA alleles used in haplotypes (NOD2/CARD15)

| | SeqID | | | | | | |
|---|---|---|---|---|---|---|---|
| | 13789 | 13796 | 13799 | 13800 | 13802 | 13804 | 13805 |
| | Position | | | | | | |
| | 49303427 | 49305205 | 49308676 | 49308899 | 49310925 | 49314041 | 49314275 |
| | Allele | | | | | | |
| RISK | T/C | T/C | T/G | A/G | A/G | G/C | T/C |
| 2121222 | C | T | G | A | G | C | C |
| 1121211 | T | T | G | A | G | G | T |
| 2121211 | C | T | G | A | G | G | T |

DNA alleles used in haplotypes (IL23R)

| | SeqID | | | | | | | | |
|---|---|---|---|---|---|---|---|---|---|
| | 5382 | 5387 | 5390 | 5393 | 5396 | 5397 | 5401 | 5404 | 5406 |
| | Position | | | | | | | | |
| | 67381537 | 67382937 | 67384786 | 67387749 | 67388943 | 67390943 | 67392949 | 67395507 | 67397137 |
| | Alleles | | | | | | | | |
| | T\|G | A\|G | A\|G | T\|G | T\|C | A\|C | T\|C | T\|C | T\|C |
| RISK | | | | | | | | | |
| 221211121 | G | G | A | G | T | A | T | C | T |
| Heterozygotes | | | | | | | | | |
| (risk carrier) | | | | | | | | | |
| 122122212 | T | G | G | T | C | C | C | T | C |
| 222122211 | G | G | G | T | C | C | C | T | T |
| 212111122 | G | A | G | T | T | A | T | C | C |
| PROTECTIVE | | | | | | | | | |
| 212111122 | G | A | G | T | T | A | T | C | C |
| 212111121 | G | A | G | T | T | A | T | C | T |

5. Singletype Analysis

The SINGLETYPE algorithm assesses the significance of case-control association for single markers using the genotype data from the laboratory as input in contrast to LDSTATS single marker window analyses, in which case-control alleles for single markers from estimated haplotypes in file, hapatctr.txt, as input. SINGLETYPE calculates P values for association for both alleles, 1 and 2, as well as for genotypes, 11, 12, and 22, and plots these as −log₁₀ P values for significance of association against marker position.

### Example 4

### Fine Mapping

Thirty one of the top regions identified as being associated with Crohn's disease by the GWS were further analyzed by fine mapping using a denser set of markers, in order to validate and/or refine the signal. The fine mapping was carried out using the Illumina BeadStation 500GX SNP genotyping platform. Alleles were genotyped using an allele-specific elongation assay that was ligated to a locus-specific oligonucleotide. The assay was performed directly on genomic DNA at a highly multiplex level and the products were amplified using universal oligonucleotides. For each candidate region, a set of SNP markers was selected with an average inter-marker distance of 1-4 Kb distributed over about 400 Kb to 1 Mb and were roughly centered at the highest point of the GWS curves. The cohort used for the fine mapping consisted of 477 Crohn's disease trios (including the 382 used for the GWS). The algorithms used for genetic analyses were the same as used in the GWS and are described in Example 3. Table 3 lists the fine mapping SNPs for the 31 confirmed regions and their respective p values using 477 trios and two analysis methods: LDSTATS (v2.0) and LDSTATS (v4.0). For each region that was associated with Crohn disease in the fine mapping analyses, we report in Table 14a and 14b the allele frequencies and the relative risk (RR) for the haplotypes contributing to the best signal at each SNP in the region. The best signal at a given location was determined by comparing the significance (p-value) of the association with Crohn disease for multiple window sizes, and selecting the most significant window. For a given window size at a given location, the association with Crohn disease was evaluated by comparing the overall distribution of haplotypes in the cases with the overall distribution of haplotypes in the controls. Haplotypes with a relative risk greater than one increase the risk of developing Crohn disease while haplotypes with a relative risk less than one are protective and decrease the risk.

Eighteen of the 31 regions fine mapped in the QFP samples were also fine mapped using samples from a European general population (both PPC trios and case control German samples collected as described in Example 1). Many of those were confirmed in both the QFP and General population (see Tables 4 and 5 for the genetic analysis results, using both LDSTATs V2 and V4 methods of analysis).

### Example 5

### Gene Identification and Characterization

A series of gene characterization was performed for each candidate region described in Table 1. Any gene or EST mapping to the interval based on public map data or proprietary map data was considered as a candidate Crohn's disease gene. The approach used to identify all genes located in the critical regions is described below.

Public Gene Mining

Once regions were identified using the analyses described above, a series of public data mining efforts were undertaken, with the aim of identifying all genes located within the critical intervals as well as their respective structural elements (i.e., promoters and other regulatory elements, UTRs, exons and splice sites). The initial analysis relied on annotation information stored in public databases (e.g. NCBI, UCSC Genome Bioinformatics, Entrez Human Genome Browser, OMIM—see below for database URL information). Table 8 lists the genes that have been mapped to the 31 candidate regions.

Database URLs

| Name | URL |
|---|---|
| Biocarta | http://www.biocarta.com/ |
| BioCyc | http://www.biocyc.org/ |
| Biomolecular Interaction | http://bind.ca/ |
| Network Database (BIND) | |
| Database of Interacting Proteins | http://dip.doe-mbi.ucla.edu/ |
| Gene Expression Omnibus | http://www.ncbi.nlm.nih.gov/geo/ |
| Human Genome Browser | http://www.ensembl.org/Homo_sapiens/ |
| Interdom | http://interdom.lit.org.sg/help/term.php |
| Kyoto Encyclopedia of Genes | http://www.genome.jp/kegg/ |
| and Genomes (KEGG) | |
| Molecular Interactions Database | http://mint.bio.uniroma2.it/mint/ |
| (MINT) | |
| National Center for | http://www.ncbi.nlm.nih.gov/ |
| Biotechnology Information | |
| (NCBI) | |
| Online Mendelian Inheritance in | http://www.ncbi.nlm.nih.gov/entrez/query.fcgi?db=OMIM |
| Man (OMIM) | |
| OmniViz | http://www.omniviz.com/applications/omni_viz.htm |
| Pathway Enterprise | http://www.omniviz.com/applications/pathways.htm |
| Reactome | http://www.reactome.org/ |
| Transpath | http://www.biobase.de/pages/products/transpath.html |
| UCSC Genome Bioinformatics | http://genome.ucsc.edu/index.html?org=Human |
| UniGene | http://www.ncbi.nlm.nih.gov/entrez/query.fcgi?db=unigene |

For some genes the available public annotation was extensive, whereas for others very little was known about a gene's function. Customized analysis was therefore performed to characterize genes that corresponded to this latter class.

Importantly, the presence of rare splice variants and artifactual ESTs was carefully evaluated. Subsequent cluster analysis of novel ESTs provided an indication of additional gene content in some cases. The resulting clusters were graphically displayed against the genomic sequence, providing indications of separate clusters that may contribute to the same gene, thereby facilitating development of confirmatory experiments in the laboratory. While much of this information was available in the public domain, the customized analysis performed revealed additional information not immediately apparent from the public genome browsers.

A unique consensus sequence was constructed for each splice variant and a trained reviewer assessed each alignment. This assessment included examination of all putative splice junctions for consensus splice donor/acceptor sequences, putative start codons, consensus Kozak sequences and upstream in-frame stops, and the location of polyadenylation signals. In addition, conserved noncoding sequences (CNSs) that could potentially be involved in regulatory functions were included as important information for each gene. The genomic reference and exon sequences were then archived for future reference. A master assembly that included all splice variants, exons and the genomic structure was used in subsequent analyses (i.e., analysis of polymorphisms). Table 9 lists gene clusters based on the publicly available EST and cDNA clustering algorithm, ECGene.

An important component of these efforts was the ability to visualize and store the results of the data mining efforts. A customized version of the highly versatile genome browser GBrowse (http://www.gmod.org/) was implemented in order to permit the visualization of several types of information against the corresponding genomic sequence. In addition, the results of the statistical analyses were plotted against the genomic interval, thereby greatly facilitating focused analysis of gene content.

Computational Analysis of Genes and GeneMaps

In order to assist in the prioritization of candidate genes for which minimal annotation existed, a series of computational analyses were performed that included basic BLAST searches and alignments to identify related genes. In some cases this provided an indication of potential function. In addition, protein domains and motifs were identified that further assisted in the understanding of potential function, as well as predicted cellular localization.

A comprehensive review of the public literature was also performed in order to facilitate identification of information regarding the potential role of candidate genes in the pathophysiology of Crohn's disease. In addition to the standard review of the literature, public resources (Medline and other online databases) were also mined for information regarding the involvement of candidate genes in specific signaling pathways. The Ingenuity Pathway Analysis System was also used to generate protein interaction networks (see above). A variety of pathway and yeast two hybrid databases were mined for information regarding protein-protein interactions. These included BIND, MINT, DIP, Interdom, and Reactome, among others. By identifying homologues of genes in the Crohn's candidate regions and exploring whether interacting proteins had been identified already, knowledge regarding the GeneMaps for Crohn's disease was advanced. The pathway information gained from the use of these resources was also integrated with the literature review efforts, as described above.

Genes identified in the WGAS and subsequent fine-mapping studies for Crohn's disease (CD) were evaluated using the Ingenuity Pathway Analysis application (IPA, Ingenuity systems) in order to identify direct biological interactions between these genes, and also to identify molecular regulators acting on those genes (indirect interactions) that could be also involved in CD. The purpose of this effort was to decipher the molecules involved in contributing to CD. These gene interaction networks are very valuable tools in the sense that they facilitate extension of the map of gene products that could represent potential drug targets for CD.

From the genetic analyses, 31 candidate regions were considered for the development of potential protein interaction networks involved in CD. These regions and their coordinates are presented in Table 1. Out of 31 regions, 4 regions were not included in this analysis because they did not contain any annotated genes. Tables 8 and 19 list the annotated genes present in the remaining 27 regions, and that were used for IPA analysis.

A total of 295 annotated genes were identified in the 27 fine-mapped regions (Tables 8 and 19), and were imported in the IPA software. In a first step, the analysis was performed by looking for direct interactions only. From this analysis 285 genes were mapped to the Ingenuity database and assigned to 23 networks as defined by IPA. These networks are based on functional relationships between gene products using known interactions in the literature. For each network, some nodes were manually extended to include good candidate genes that could play a role in the biochemical pathways of CD. Table 20 contains information about the gene content of each network, as well as the top functions assigned to those biochemical pathways.

In a second step, the analysis was performed by looking for direct and indirect interactions. From this analysis 270 genes were mapped to the Ingenuity database and assigned to 17 genetic networks as defined by IPA. Table 21 contains information about the gene content of each network, as well as the top functions assigned to those biochemical pathways.

In a third step, a subset of the genes (61) mapping to the candidate regions was used as input to the Ingenuity Pathway Analysis System (Table 22). These genes were selected according to criteria that included their relevance to the pathophysiology of the disease and location with respect to the statistical evidence. Tables 23 and 24 list the networks derived from direct interaction only as well as direct and indirect interaction conditions.

Expression Studies

In order to determine the expression patterns for genes, relevant information was first extracted from public databases. The UniGene database, for example, contains information regarding the tissue source for ESTs and cDNAs contributing to individual clusters. This information was extracted and summarized to provide an indication in which tissues the gene was expressed. Particular emphasis was placed on annotating the tissue source for bona fide ESTs, since many ESTs mapped to Unigene clusters are artifactual. In addition, SAGE and microarray data, also curated at NCBI (Gene Expression Omnibus), provided information on expression profiles for individual genes. Particular emphasis was placed on identifying genes that were expressed in tissues known to be involved in the pathophysiology of Crohn's disease, e.g. intestinal and immune system tissues.

Polymorphism Analysis

Polymorphisms identified in candidate genes, including those from the public domain as well as those identified by sequencing candidate genes and regions, are evaluated for potential function. Initially, polymorphisms are examined for potential impact upon encoded proteins. If the protein is a member of a gene family with reported 3-dimensional structural information, this information is used to predict the location of the polymorphism with respect to protein structure. This information provided insight into the potential role of polymorphisms in altering protein or ligand interactions, as well as suitability as a drug target. In a second phase of analysis we evaluated the potential role of polymorphisms in other biological phenomena, including regulation of transcription, splicing and mRNA stability, etc. There are many examples of the functional involvement of naturally occurring polymorphisms in these processes. As part of this analysis, polymorphisms located in promoter or other regulatory elements, canonical splice sites, exonic and intronic splice enhancers and repressors, conserved noncoding sequences and UTRs are localized.

### Example 6

### SNP and Polymorphism Discovery (SNPD)

Three candidate regions were selected for sequencing in order to identify all polymorphisms. In cases where the critical interval, identified by fine mapping, was relatively small (˜50 kb), the entire region, including all introns, was sequenced to identify polymorphisms. In situations where the region is large (>50 kb), candidate genes are prioritized for sequencing, and/or only functional gene elements (promoters, exons and splice sites) are sequenced.

The samples sequenced are selected according to which haplotypes contribute to the association signal observed in the region. The purpose is to select a set of samples that covered all the major haplotypes in the given region. Each major haplotype must be present in a few copies. The first step therefore consisted of determining the major haplotypes in the region to be sequenced.

Once a region was defined with the two boundary markers, all the markers used in fine mapping that are located within the region are used to determine the major haplotypes. Long haplotypes covering the whole region are thus inferred using the middle marker as an anchor. The results included two series of haplotype themes that define the major haplotypes, comparing the cases and the controls. This exercise was repeated using an anchor in the peripheral regions to ensure that major haplotype subsets that are not anchored at the original middle marker are not missed.

Once the major haplotypes were determined as described above, appropriate genomic DNA samples were selected such that each major haplotype and haplotype subset were represented in at least two to four copies.

The sequencing protocol included the following steps, once a region was delimited:

1. Primer Design

The design of the primers was performed using a proprietary primer design tool. A primer quality control step was included in the primer design process. Primers that successfully passed the control quality process were synthesized by Integrated DNA Technologies (IDT). The sense and anti-sense oligos were separated such that the sense oligos were placed on one plate in the same position as their anti-sense counterparts on another plate. Two additional plates were created from each storage plate, one for use in PCR and the other for sequencing. For PCR, the sense and anti-sense oligos of the same pair were combined in the same well to achieve a final concentration of 1.5 μM for each oligonucleotide.

2. PCR Optimization

PCR conditions were optimized by testing a variety of conditions that included varying salt concentrations and temperatures, as well as including various additives. PCR products were checked for robust amplification and minimal background by agarose gel electrophoresis.

3. PCR on Selected Samples

PCR products used for sequencing were amplified using the conditions chosen during optimization. The PCR products were purified free of salts, dNTPs and unincorporated primers by use of a MultiScreen PCR384 filter plate manufactured by Millipore. Following PCR, the amplicons were quantified by use of a lambda/Hind III standard curve. This was done to ensure that the quantity of PCR product required for sequencing had been generated. The raw data was measured against the standard curve data in Excel by use of a macro.

4. Sequencing

Sequencing of PCR products was performed by DNA Landmarks using ABI 3730 capillary sequencing instruments.

5. Sequence Analysis

The ABI Prism SeqScape software (Applied Biosystems) was used for SNP identification. The chromatogram trace files were imported into a SeqScape sequencing project and the base calling was automatically performed. Sequences are then aligned and compared to each other using the SeqScape program. The base calling was checked manually, base by base; editing was performed if needed. The SNPs and polymorphisms discovered in this example are listed in Table 10.

### Example 7

### Ultra Fine Mapping (UFM)

Once polymorphisms were identified by sequencing efforts as described in Example 6, additional genotyping of all newly found polymorphisms was performed on the samples used in the fine mapping studies. Various types of genotyping assays may need to be utilized based on the type of polymorphism identified (i.e., SNP, indel, microsatellite). The assay type can be, but is not restricted to, Sentrix Assay Matrix on Illumina BeadStations, microsatellite on MegaBACE, SNP on ABI or Orchid. The frequencies of genotypes and haplotypes in cases and controls were analyzed in a similar manner as the GWS and fine mapping data. By examining all SNPs in a region, polymorphisms are identified that increase an individual's susceptibility to Crohn's disease. The goal of ultra-fine mapping was to identify the polymorphism that is most associated with disease phenotype as part of the search for the actual DNA polymorphism that confers susceptibility to disease. This statistical identification may need to be corroborated by functional studies. Table 6 lists the results for ultrafine mapping of 3 regions in the QFP. For each region that was associated with Crohn disease in the ultrafine mapping analyses in the QFP, we report in Table 17a and 17b the allele frequencies and the relative risk (RR) for the haplotypes contributing to the best signal at each SNP in the region. The best signal at a given location was determined by comparing the significance (p-value) of the association with Crohn disease for multiple window sizes, and selecting the most significant window. For a given window size at a given location, the association with Crohn disease was evaluated by comparing the overall distribution of haplotypes in the cases with the overall distribution of haplotypes in the controls. Haplotypes with a relative risk greater than one increase the risk of developing Crohn disease while haplotypes with a relative risk less than one are protective and decrease the risk.

### Example 8

### Confirmation of Candidate Regions and Genes in a General Population

The confirmation of any putative associations described in Examples 4 and 7 was performed in an independent North-European patient sample (see Examples 1 and 3 for description of analysis and samples used). These DNA samples consist of two cohorts. The first cohort consists of 500 PPC trios (500 patients with Crohn's disease). The second cohort consists of 750 patients having Crohn's disease and 750 controls. Haplotype information surrounding association signals are derived by exploration of the Falk-Rubinstein-Trios of North European descent. The fine mapping results for replication of 18 candidate regions in the North-European trios and case/controls are listed in Tables 4 and 5. For each region that was associated with Crohn disease in the fine mapping analyses of the North-European trios and case.controls, we report in Tables 15a and 15b and 16a and 16b the allele frequencies and the relative risk (RR) for the haplotypes contributing to the best signal at each SNP in the region, for both cohorts. The best signal at a given location was determined by comparing the significance (p-value) of the association with Crohn disease for multiple window sizes, and selecting the most significant window. For a given window size at a given location, the association with Crohn disease was evaluated by comparing the overall distribution of haplotypes in the cases with the overall distribution of haplotypes in the controls. Haplotypes with a relative risk greater than one increase the risk of developing Crohn disease while haplotypes with a relative risk less than one are protective and decrease the risk.

The ultrafine mapping results for replication of three candidate regions in the North-European trios are listed in Table 7. For each region that was associated with Crohn disease in the ultrafine mapping analyses of the North-European trios, we report in Tables 18a and 18b the allele frequencies and the relative risk (RR) for the haplotypes contributing to the best signal at each SNP in the region. The best signal at a given location was determined by comparing the significance (p-value) of the association with Crohn disease for multiple window sizes, and selecting the most significant window. For a given window size at a given location, the association with Crohn disease was evaluated by comparing the overall distribution of haplotypes in the cases with the overall distribution of haplotypes in the controls. Haplotypes with a relative risk greater than one increase the risk of developing Crohn disease while haplotypes with a relative risk less than one are protective and decrease the risk.

All publications, patents and patent applications mentioned in the specification and reference list are herein incorporated by reference in their entirety for all purposes. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, or related fields are intended to be within the scope of the following claims.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and H (D. N. Glover ed., 4); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Haines & S. J. Higgins eds. 1984); Culture Of Animal Cells (R. 1. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### REFERENCES

Aaltonen, J., M. P. Laitinen, et al. (1999). “Human growth differentiation factor 9 (GDF-9) and its novel homolog GDF-9B are expressed in oocytes during early folliculogenesis.” J Clin Endocrinol Metab. 84(8): 2744-50.

Abbott, D. W., A. Wilkins, et al. (2004). “The Crohn's Disease Protein, NOD2, Requires RIP2 in Order to Induce Ubiquitinylation of a Novel Site on NEMO.” Curr Biol 14(24): 2217-27.

Adcock, I. M., M. J. Peters, et al. (1993). “Transcription factor interactions in human lung.” Biochem Soc Trans 21(Pt 3)(3): 277S.

Agnholt et al (2004). Increased production of granulocyte-macrophage colony-stimulating factor in Crohn's disease—a possible target for infliximab treatment. Eur J Gastroenterol Hepatol. 16(7):649-55.

Aithal, G. P., C. P. Day, et al. (2001). “Association of single nucleotide polymorphisms in the interleukin-4 gene and interleukin-4 receptor gene with Crohn's disease in a British population.” Genes Immun 2(1): 44-7.

Alvarado-Kristensson, M. and T. Andersson (2005). “Protein phosphatase 2A regulates apoptosis in neutrophils by dephosphorylating both p38 MAPK and its substrate caspase 3.” J Biol Chem. 280(7): 6238-44.

Alwine, J. C., D. J. Kemp, et al. (1977). “Method for detection of specific RNAs in agarose gels by transfer to diazobenzyloxymethyl-paper and hybridization with DNA probes.” Proc Natl Acad Sci USA 74(12): 5350-4.

Anderson, W. F. (1992). “Human gene therapy.” Science 256(5058): 808-13.

Andres, P. G. and L. S. Friedman (1999). “Epidemiology and the natural course of inflammatory bowel disease.” Gastroenterol Clin North Am 28(2): 255-81, vii.

Angeloni, D., F. M. Duh, et al. (2003). “C to A single nucleotide polymorphism in intron 18 of the human MST1R (RON) gene that maps at 3p21.3.” Mol Cell Probes. 17(2-3): 55-7.

Aoki et al (1995). A novel gene, Translin, encodes a recombination hotspot binding protein associated with chromosomal translocations. Nat Genet. 10(2):167-74.

Bai, R. Y., C. Koester, et al. (2002). “SMIF, a Smad4-interacting protein that functions as a co-activator in TGFbeta signaling.” Nat Cell Biol. 4(3): 181-90.

Barany, F. (1991). “Genetic disease detection and DNA amplification using cloned thermostable ligase.” Proc Natl Acad Sci USA 88(1): 189-93.

Barre, F. X., S. Ait-Si-Ali, et al. (2000). “Unambiguous demonstration of triple-helix-directed gene modification.” Proc Natl Acad Sci USA 97(7): 3084-8.

Becker, C., S. Wirtz, et al. (2003). “Constitutive p40 promoter activation and IL-23 production in the terminal ileum mediated by dendritic cells.” J Clin Invest 112(5): 693-706.

Bednarek, A. K., C. L. Keck-Waggoner, et al. (2001). “WWOX, the FRA16D gene, behaves as a suppressor of tumor growth.” Cancer Res 61(22): 8068-73.

Behl, C. (1997). “Amyloid beta-protein toxicity and oxidative stress in Alzheimer's disease.” Cell Tissue Res 290(3): 471-80.

Belkhiri, A., A. Zaika, et al. (2005). “Darpp-32: a novel antiapoptotic gene in upper gastrointestinal carcinomas.” Cancer Res. 65(15): 6583-92.

Bender, C. F., M. L. Sikes, et al. (2002). “Cancer predisposition and hematopoietic failure in Rad50(S/S) mice.” Genes Dev. 16(17): 2237-51.

Bespalova, I. N., G. Van Camp, et al. (2001). “Mutations in the Wolfram syndrome 1 gene (WFS1) are a common cause of low frequency sensorineural hearing loss.” Hum Mol Genet 10(22): 2501-8.

Bielenberg, D. R., Y. Hida, et al. (2004). “Semaphorin 3F, a chemorepulsant for endothelial cells, induces a poorly vascularized, encapsulated, nonmetastatic tumor phenotype.” J Clin Invest. 114(9): 1260-71.

Bjursten, M., P. W. Bland, et al. (2005). “Long-term treatment with anti-alpha 4 integrin antibodies aggravates colitis in G alpha i2-deficient mice.” Eur J Immunol. 35(8): 2274-83.

Blanchard, C., S. Durual, et al. (2004). “IL-4 and IL-13 up-regulate intestinal trefoil factor expression: requirement for STAT6 and de novo protein synthesis.” J Immunol 172(6): 3775-83.

Blaser, S., J. Horn, et al. (2004). “The novel human platelet septin SEPT8 is an interaction partner of SEPT4.” Thromb Haemost. 91(5): 959-66.

Bloch, K. D., J. R. Wolfram, et al. (1995). “Three members of the nitric oxide synthase II gene family (NOS2A, NOS2B, and NOS2C) colocalize to human chromosome 17.” Genomics 27(3): 526-30.

Boengler, K., F. Pipp, et al. (2003). “The ankyrin repeat containing SOCS box protein 5: a novel protein associated with arteriogenesis.” Biochem Biophys Res Commun 302(1): 17-22.

Boren, J., A. Ramos-Montoya, et al. (2006). “In situ localization of transketolase activity in epithelial cells of different rat tissues and subcellularly in liver parenchymal cells.” J Histochem Cytochem. 54(2): 191-9.

Bouma, G. and W. Strober (2003). “The immunological and genetic basis of inflammatory bowel disease.” Nat Rev Immunol 3(7): 521-33.

Bourgeois, S. and D. Labuda (2004). “Dynamic allele-specific oligonucleotide hybridization on solid support.” Anal Biochem 324(2): 309-11.

Brightbill, H. D., D. H. Libraty, et al. (1999). “Host defense mechanisms triggered by microbial lipoproteins through toll-like receptors.” Science 285(5428): 732-6.

Brodbeck, J., A. Davies, et al. (2002). “The ducky mutation in Cacna2d2 results in altered Purkinje cell morphology and is associated with the expression of a truncated alpha 2 delta-2 protein with abnormal function.” J Biol Chem. 277(10): 7684-93.

Brown, J. L., L. Stowers, et al. (1996). “Human Ste20 homologue hPAK1 links GTPases to the JNK MAP kinase pathway.” Curr Biol 6(5): 598-605.

Browning, C. M., M. J. Smith, et al. (2001). “Human GLI-2 is a tat activation response element-independent Tat cofactor.” J Virol 75(5): 2314-23.

Bulavin, D. V., C. Phillips, et al. (2004). “Inactivation of the Wip1 phosphatase inhibits mammary tumorigenesis through p38 MAPK-mediated activation of the p16(Ink4a)-p19(Arf) pathway.” Nat Genet 36(4): 343-50.

Cai, D., L. K. Clayton, et al. (1999). “AND-34, a novel p130Cas-binding thymic stromal cell protein regulated by adhesion and inflammatory cytokines.” J Immunol. 163(4): 2104-12.

Cai, D., K. N. Felekkis, et al. (2003). “The GDP exchange factor AND-34 is expressed in B cells, associates with HEF1, and activates Cdc42.” J Immunol. 170(2): 969-78.

Cale, J. M., C. E. Shaw, et al. (1998). “Optimization of a reverse transcription-polymerase chain reaction (RT-PCR) mass assay for low-abundance mRNA.” Methods Mol Biol 105: 351-71.

Chamouard, P., L. Grunebaum, et al. (1995). “Prothrombin fragment 1+2 and thrombin-antithrombin III complex as markers of activation of blood coagulation in inflammatory bowel diseases.” Eur J Gastroenterol Hepatol. 7(12): 1183-8.

Chang, N. S., N. Pratt, et al. (2001). “Hyaluronidase induction of a WW domain-containing oxidoreductase that enhances tumor necrosis factor cytotoxicity.” J Biol Chem 276(5): 3361-70.

Chavany, C., C. Vicario-Abejon, et al. (1998). “Transgenic mice for interleukin 3 develop motor neuron degeneration associated with autoimmune reaction against spinal cord motor neurons.” Proc Natl Acad Sci USA 95(19): 11354-9.

Chen, X., L. Levine, et al. (1999). “Fluorescence polarization in homogeneous nucleic acid analysis.” Genome Res 9(5): 492-8.

Chen, S., X. Yin, et al. (2003). “The C-terminal kinase domain of the p34cdc2-related PITSLRE protein kinase (p110C) associates with p21-activated kinase 1 and inhibits its activity during anoikis.” J Biol Chem 278(22): 20029-36. Epub 2003 Mar. 6.

Chen, Q., L. Rabach, et al. (2005). “IL-11 receptor alpha in the pathogenesis of IL-13-induced inflammation and remodeling.” J Immunol 174(4): 2305-13.

Chien, C. T., P. L. Bartel, et al. (1991). “The two-hybrid system: a method to identify and clone genes for proteins that interact with a protein of interest.” Proc Natl Acad Sci USA 88(21): 9578-82.

Choi, J., B. Nannenga, et al. (2002). “Mice deficient for the wild-type p53-induced phosphatase gene (Wip1) exhibit defects in reproductive organs, immune function, and cell cycle control.” Mol Cell Biol 22(4): 1094-105.

Clavell, M., H. Correa-Gracian, et al. (2000). “Detection of interferon regulatory factor-1 in lamina propria mononuclear cells in Crohn's disease.” J Pediatr Gastroenterol Nutr. 30(1): 43-7.

Cobrin, G. M. and M. T. Abreu (2005). “Defects in mucosal immunity leading to Crohn's disease.” Immunol Rev. 206: 277-95.

Cohen, A. S., D. L. Smisek, et al. (1996). “Emerging technologies for sequencing antisense oligonucleotides: capillary electrophoresis and mass spectrometry.” Adv Chromatogr 36: 127-62.

Cohn, R. D., M. D. Henry, et al. (2002). “Disruption of DAG1 in differentiated skeletal muscle reveals a role for dystroglycan in muscle regeneration.” Cell 110(5): 639-48.

Combaret, L., O. A. Adegoke, et al. (2005). “USP19 is a ubiquitin-specific protease regulated in rat skeletal muscle during catabolic states.” Am J Physiol Endocrinol Metab. 288(4): E693-700.

Costello et al (2005). Dissection of the inflammatory bowel disease transcriptome using genome-wide cDNA microarrays. PLoS Med. August; 2(8):e199.

Cotton, R. G., N. R. Rodrigues, et al. (1988). “Reactivity of cytosine and thymine in single-base-pair mismatches with hydroxylamine and osmium tetroxide and its application to the study of mutations.” Proc Natl Acad Sci USA 85(12): 4397-401.

Couve, A., S. Restituito, et al. (2004). “Marlin-1, a novel RNA-binding protein associates with GABA receptors.” J Biol Chem 279(14): 13934-43. Epub 2004 Jan. 12.

Couzens, M., M. Liu, at al. (2000). “Peptide YY-2 (PYY2) and pancreatic polypeptide-2 (PPY2): species-specific evolution of novel members of the neuropeptide Y gene family.” Genomics 64(3): 318-23.

Cronin, M. T., R. V. Fucini, et al. (1996). “Cystic fibrosis mutation detection by hybridization to light-generated DNA probe arrays.” Hum Mutat 7(3): 244-55.

Croucher, P. J., S. Mascheretti, et al. (2003). “Lack of association between the C3435T MDR1 gene polymorphism and inflammatory bowel disease in two independent Northern European populations.” Gastroenterology 125(6): 1919-20; author reply 1920-1.

Cuppen, E., M. van Ham, et al. (2000). “The zyxin-related protein TRIP6 interacts with PDZ motifs in the adaptor protein RIL and the protein tyrosine phosphatase PTP-BL.” Eur J Cell Biol. 79(4): 283-93.

Dalwadi, H., B. Wei, et al. (2003). “B cell developmental requirement for the G alpha i2 gene.” J Immunol. 170(4): 1707-15.

Dean, F. B., S. Hosono, et al. (2002). “Comprehensive human genome amplification using multiple displacement amplification.” Proc Natl Acad Sci USA 99(8): 5261-6.

del Peso, L., R. Hernandez-Alcoceba, et al. (1997). “Rho proteins induce metastatic properties in vivo.” Oncogene 15(25): 3047-57.

Dentelli, P., A. Rosso, et al. (2004). “IL-3 affects endothelial cell-mediated smooth muscle cell recruitment by increasing TGF beta activity: potential role in tumor vessel stabilization.” Oncogene 23(9): 1681-92.

DeSalle, L. M., E. Latres, et al. (2001). “The de-ubiquitinating enzyme Unp interacts with the retinoblastoma protein.” Oncogene. 20(39): 5538-42.

Deschenes, C., L. Alvarez, et al. (2004). “The nucleocytoplasmic shuttling of E2F4 is involved in the regulation of human intestinal epithelial cell proliferation and differentiation.” J Cell Physiol. 199(2): 262-73.

Diefenbach, A., H. Schindler, et al. (1999). “Requirement for type 2 NO synthase for IL-12 signaling in innate immunity.” Science 284(5416): 951-5.

Dijkstra, G., A. J. Zandvoort, et al. (2002). “Increased expression of inducible nitric oxide synthase in circulating monocytes from patients with active inflammatory bowel disease.” Scand J Gastroenterol. 37(5): 546-54.

Dillon, N. (1993). “Regulating gene expression in gene therapy.” Trends Biotechnol 11(5): 167-73.

Ding, Z., L. L. Gillespie, et al. (2003). “Human MI-ER1 alpha and beta function as transcriptional repressors by recruitment of histone deacetylase 1 to their conserved ELM2 domain.” Mol Cell Biol. 23(1): 250-8.

Donato, J. L., J. Ko, et al. (2002). “Human HTm4 is a hematopoietic cell cycle regulator.” J Clin Invest. 109(1): 51-8.

Donovan, F. M., C. J. Pike, et al. (1997). “Thrombin induces apoptosis in cultured neurons and astrocytes via a pathway requiring tyrosine kinase and RhoA activities.” J Neurosci 17(14): 5316-26.

Dranoff, G., R. Soiffer, et al. (1997). “A phase I study of vaccination with autologous, irradiated melanoma cells engineered to secrete human granulocyte-macrophage colony stimulating factor.” Hum Gene Ther 8(1): 111-23.

Drmanac, R., S. Drmanac, et al. (2002). “Sequencing by hybridization (SBH): advantages, achievements, and opportunities.” Adv Biochem Eng Biotechnol 77: 75-101.

Dryja, T. P., L. B. Hahn, et al. (1996). “Missense mutation in the gene encoding the alpha subunit of rod transducin in the Nougaret form of congenital stationary night blindness.” Nat Genet. 13(3): 358-60.

Ducale, A. E., S. I. Ward, et al. (2005). “Regulation of hyaluronan synthase-2 expression in human intestinal mesenchymal cells: mechanisms of interleukin-1beta-mediated induction.” Am J Physiol Gastrointest Liver Physiol. 289(3): G462-70.

Duerr, R. H. (2002). “The genetics of inflammatory bowel disease.” Gastroenterol Clin North Am 31(1): 63-76.

Dunbar, C. E., M. Cottler-Fox, et al. (1995). “Retrovirally marked CD34-enriched peripheral blood and bone marrow cells contribute to long-term engraftment after autologous transplantation.” Blood 85(11): 3048-57.

Dusetti, N. J., Y. Jiang, et al. (2002). “Cloning and expression of the rat vacuole membrane protein 1 (VMP1), a new gene activated in pancreas with acute pancreatitis, which promotes vacuole formation.” Biochem Biophys Res Commun 290(2): 641-9.

Elez, R., A. Piiper, et al. (2000). “Polo-like kinase1, a new target for antisense tumor therapy.” Biochem Biophys Res Commun 269(2): 352-6.

Ellem, K. A., M. G. O'Rourke, et al. (1997). “A case report: immune responses and clinical course of the first human use of granulocyte/macrophage-colony-stimulating-factor-transduced autologous melanoma cells for immunotherapy.” Cancer Immunol Immunother 44(1): 10-20.

Esaki, M., M. Furuse, et al. (1999). “Polymorphism of heat-shock protein gene HSP70-2 in Crohn disease: possible genetic marker for two forms of Crohn disease.” Scand J Gastroenterol 34(7): 703-7.

Esworthy R. S., R. Aranda, et. (2001). “Mice with combined disruption of Gpx1 and Gpx2 genes have colitis.” Am J Physiol Gastrointest Liver Physiol. 281(3):G848-55. PMID: 11518697.

Evans, B. E., K. E. Rittle, et al. (1987). “Design of nonpeptidal ligands for a peptide receptor: cholecystokinin antagonists.” J Med Chem 30(7): 1229-39.

Fackler, O. T. and B. M. Peterlin (2000). “Endocytic entry of HIV-1.” Curr Biol 10(16): 1005-8.

Fakhrai-Rad, H., J. Zheng, et al. (2004). “SNP discovery in pooled samples with mismatch repair detection.” Genome Res 14(7): 1404-12.

Fan, J. and A. B. Malik (2003). “Toll-like receptor-4 (TLR4) signaling augments chemokine-induced neutrophil migration by modulating cell surface expression of chemokine receptors.” Nat Med 9(3): 315-21.

Farmer, R. G., G. Whelan, et al. (1985). “Long-term follow-up of patients with Crohn's disease. Relationship between the clinical pattern and prognosis.” Gastroenterology 88(6): 1818-25.

Fechir, M., K. Linker, et al. (2005). “The RNA binding protein TIAR is involved in the regulation of human iNOS expression.” Cell Mol Biol (Noisy-le-grand). 51(3): 299-305.

Fidder, H. H., S. Olschwang, et al. (2003). “Association between mutations in the CARD15 (NOD2) gene and Crohn's disease in Israeli Jewish patients.” Am J Med Genet 121A(3): 240-4.

Fields, S. and O. Song (1989). “A novel genetic system to detect protein-protein interactions.” Nature 340(6230): 245-6.

Frank-Kamenetskii, M. D. and S. M. Mirkin (1995). “Triplex DNA structures.” Annu Rev Biochem 64: 65-95.

Freeman, W. M., S. J. Walker, et al. (1999). “Quantitative RT-PCR: pitfalls and potential.” Biotechniques 26(1): 112-22, 124-5.

Frolova, E. I., G. M. Dolganov, et al. (1991). “Linkage mapping of the human CSF2 and IL3 genes.” Proc Natl Acad Sci USA 88(11): 4821-4.

Fujita, Y., Y. Ezura, et al. (2004). “Hypercholesterolemia associated with splice-junction variation of inter-alpha-trypsin inhibitor heavy chain 4 (ITIH4) gene.” J Hum Genet 49(1): 24-8.

Fukushige, S., K. Matsubara, et al. (1986). “Localization of a novel v-erbB-related gene, c-erbB-2, on human chromosome 17 and its amplification in a gastric cancer cell line.” Mol Cell Biol 6(3): 955-8.

Fukushima, T., J. M. Zapata, et al. (2006). “Critical function for SIP, a ubiquitin E3 ligase component of the beta-catenin degradation pathway, for thymocyte development and G1 checkpoint.” Immunity. 24(1): 29-39.

Gainetdinov, R. R., L. M. Bohn, et al. (1999). “Muscarinic supersensitivity and impaired receptor desensitization in G protein-coupled receptor kinase 5-deficient mice.” Neuron. 24(4): 1029-36.

Gan, B., Z. K. Melkoumian, et al. (2005). “Identification of FIP200 interaction with the TSC1-TSC2 complex and its role in regulation of cell size control.” J Cell Biol. 170(3): 379-89.

Gary-Gouy, H., J. Harriague, et al. (2002). “CD5-negative regulation of B cell receptor signaling pathways originates from tyrosine residue Y429 outside an immunoreceptor tyrosine-based inhibitory motif.” J Immunol. 168(1): 232-9.

Gasparini, P., A. Bonizzato, et al. (1992). “Restriction site generating-polymerase chain reaction (RG-PCR) for the probeless detection of hidden genetic variation: application to the study of some common cystic fibrosis mutations.” Mol Cell Probes 6(1): 1-7.

Geyer, M., H. Yu, et al. (2002). “Subunit H of the V-ATPase binds to the medium chain of adaptor protein complex 2 and connects Nef to the endocytic machinery.” J Biol Chem 277(32): 28521-9. Epub 2002 May 24.

Giachino, D., M. M. van Duist, et al. (2004). “Analysis of the CARD15 variants R702W, G908R and L1007fs in Italian IBD patients.” Eur J Hum Genet 12(3): 206-12.

Gibbs, R. A., P. N. Nguyen, et al. (1989). “Detection of single DNA base differences by competitive oligonucleotide priming.” Nucleic Acids Res 17(7): 2437-48.

Girardin, S. E., I. G. Boneca, et al. (2003). “Nod2 is a general sensor of peptidoglycan through muramyl dipeptide (MDP) detection.” J Biol Chem 278(11): 8869-72.

Girardin, S. E., J. P. Hugot, et al. (2003). “Lessons from Nod2 studies: towards a link between Crohn's disease and bacterial sensing.” Trends Immunol 24(12): 652-8.

Griffin, H. G. and A. M. Griffin (1993). “DNA sequencing. Recent innovations and future trends.” Appl Biochem Biotechnol 38(1-2): 147-59.

Grossman, P. D., W. Bloch, et al. (1994). “High-density multiplex detection of nucleic acid sequences: oligonucleotide ligation assay and sequence-coded separation.” Nucleic Acids Res 22(21): 4527-34.

Guatelli, J. C., K. M. Whitfield, et al. (1990). “Isothermal, in vitro amplification of nucleic acids by a multienzyme reaction modeled after retroviral replication.” Proc Natl Acad Sci USA 87(5): 1874-8.

Guatelli, J. C., K. M. Whitfield, et al. (1990). “Isothermal, in vitro amplification of nucleic acids by a multienzyme reaction modeled after retroviral replication.” Proc Natl Acad Sci USA 87(19): 7797.

Hafner, G. J., I. C. Yang, et al. (2001). “Isothermal amplification and multimerization of DNA by Bst DNA polymerase.” Biotechniques 30(4): 852-6, 858, 860 passim.

Hampe, J., J. Grebe, et al. (2002). “Association of NOD2 (CARD 15) genotype with clinical course of Crohn's disease: a cohort study.” Lancet 359(9318): 1661-5.

Harada, M., Y. Qin, et al. (2005). “G-CSF prevents cardiac remodeling after myocardial infarction by activating the Jak-Stat pathway in cardiomyocytes.” Nat Med. 11(3): 305-11.

Hardenbol, P., J. Baner, et al. (2003). “Multiplexed genotyping with sequence-tagged molecular inversion probes.” Nat Biotechnol 21(6): 673-8. Epub 2003 May 5.

Hardwick, J. C., G. R. Van Den Brink, et al. (2004). “Bone morphogenetic protein 2 is expressed by, and acts upon, mature epithelial cells in the colon.” Gastroenterology. 126(1): 111-21.

Hartman, M. E., J. C. O'Connor, et al. (2004). “Insulin receptor substrate-2-dependent interleukin-4 signaling in macrophages is impaired in two models of type 2 diabetes mellitus.” J Biol Chem 279(27): 28045-50.

Hasegawa, T., A. Yagi, et al. (2000). “Interaction between GADD34 and kinesin superfamily, KIF3A.” Biochem Biophys Res Commun. 267(2): 593-6.

Hayashi, K. (1992). “PCR-SSCP: a method for detection of mutations.” Genet Anal Tech Appl 9(3): 73-9.

Heintz, K., K. Palme, et al. (1992). “The Ncypt1 gene from Neurospora crassa is located on chromosome 2: molecular cloning and structural analysis.” Mol Gen Genet 235(2-3): 413-21.

Hellevuo, K., M. Yoshimura, et al. (1993). “A novel adenylyl cyclase sequence cloned from the human erythroleukemia cell line.” Biochem Biophys Res Commun. 192(1): 311-8.

Helms, C., L. Cao, et al. (2003). “A putative RUNX1 binding site variant between SLC9A3R1 and NAT9 is associated with susceptibility to psoriasis.” Nat Genet 35(4): 349-56.

Henry, M. D. and K. P. Campbell (1998). “A role for dystroglycan in basement membrane assembly.” Cell. 95(6): 859-70.

Hermonat, P. L. and N. Muzyczka (1984). “Use of adeno-associated virus as a mammalian DNA cloning vector: transduction of neomycin resistance into mammalian tissue culture cells.” Proc Natl Acad Sci USA 81(20): 6466-70.

Hirai, H., K. Tanaka, et al. (2002). “Cutting edge: agonistic effect of indomethacin on a prostaglandin D2 receptor, CRTH2.” J Immunol. 168(3): 981-5.

Hisamatsu, T., M. Suzuki, et al. (2003). “Interferon-gamma augments CARD4/NOD1 gene and protein expression through interferon regulatory factor-1 in intestinal epithelial cells.” J Biol Chem. 278(35): 32962-8.

Hobbs, M. R., V. Udhayakumar, et al. (2002). “A new NOS2 promoter polymorphism associated with increased nitric oxide production and protection from severe malaria in Tanzanian and Kenyan children.” Lancet 360(9344): 1468-75.

Hoffman, J., F. Kuhnert, et al. (2004). “Wnts as essential growth factors for the adult small intestine and colon.” Cell Cycle. 3(5): 554-7.

Hogrefe, H. H., R. L. Mullinax, et al. (1993). “A bacteriophage lambda vector for the cloning and expression of immunoglobulin Fab fragments on the surface of filamentous phage.” Gene 128(1): 119-26.

Hoogendoorn, B., N. Norton, et al. (2000). “Cheap, accurate and rapid allele frequency estimation of single nucleotide polymorphisms by primer extension and DHPLC in DNA pools.” Hum Genet 107(5): 488-93.

Hsu, I. C., Q. Yang, et al. (1994). “Detection of DNA point mutations with DNA mismatch repair enzymes.” Carcinogenesis 15(8): 1657-62.

Hu, W., F. Yin, et al. (2002). “[Effect of human calcyclin binding protein encoding gene on development of multiple drug resistance in gastric cancer].” Zhonghua Zhong Liu Za Zhi. 24(5): 426-9.

Hugot, J. P., M. Chamaillard, et al. (2001). “Association of NOD2 leucine-rich repeat variants with susceptibility to Crohn's disease.” Nature 411(6837): 599-603.

Hugot, J. P. and G. Thomas (1998). “Genome-wide scanning in inflammatory bowel diseases.” Dig Dis 16(6): 364-9.

Hunt, T. W., T. A. Fields, et al. (1996). “RGS10 is a selective activator of G alpha i GTPase activity.” Nature. 383(6596): 175-7.

Hutchings, N. J., N. Clarkson, et al. (2003). “Linking the T cell surface protein CD2 to the actin-capping protein CAPZ via CMS and CIN85.” J Biol Chem. 278(25): 22396-403.

Ince-Dunn, G., B. J. Hall, et al. (2006). “Regulation of thalamocortical patterning and synaptic maturation by NeuroD2.” Neuron. 49(5): 683-95.

Inostroza, J. A., F. H. Mermelstein, et al. (1992). “Dr1, a TATA-binding protein-associated phosphoprotein and inhibitor of class II gene transcription.” Cell. 70(3): 477-89.

Irvine, E. J. (2004). “Patients' fears and unmet needs in inflammatory bowel disease.” Aliment Pharmacol Ther 20 Suppl 4: 54-9.

Ishibashi, K., M. Suzuki, et al. (2001). “Identification of a new multigene four-transmembrane family (MS4A) related to CD20, HTm4 and beta subunit of the high-affinity IgE receptor.” Gene 264(1): 87-93.

Ishigaki, S., J. Niwa, et al. (2000). “Two novel genes, human neugrin and mouse m-neugrin, are upregulated with neuronal differentiation in neuroblastoma cells.” Biochem Biophys Res Commun 279(2): 526-33.

Ishii, M., H. Fei, et al. (2003). “Targeted disruption of GPR7, the endogenous receptor for neuropeptides B and W, leads to metabolic defects and adult-onset obesity.” Proc Natl Acad Sci USA 100(18): 10540-5. Epub 2003 Aug. 18.

Ishitani, T., S. Kishida, et al. (2003). “The TAK1-NLK mitogen-activated protein kinase cascade functions in the Wnt-5a/Ca(2+) pathway to antagonize Wnt/beta-catenin signaling.” Mol Cell Biol 23(1): 131-9.

Itoh et al (2001). “Decreased Bax expression by mucosal T cells favours resistance to apoptosis in Crohn's disease.” Gut. 49(1):35-41.

Ivanov, A. I., A. Nusrat, et al. (2004). “The epithelium in inflammatory bowel disease: potential role of endocytosis of junctional proteins in barrier disruption.” Novartis Found Symp 263: 115-24.

Jan, Y., M. Matter, et al. (2004). “A mitochondrial protein, Bit1, mediates apoptosis regulated by integrins and Groucho/TLE corepressors.” Cell 116(5): 751-62.

Jandrig et al (2004). ST18 is a breast cancer tumor suppressor gene at human chromosome 8q11.2. Oncogene. 23(57):9295-302.

Jiang, P. H., Y. Motoo, et al. (2004). “Expression of vacuole membrane protein 1 (VMP1) in spontaneous chronic pancreatitis in the WBN/Kob rat.” Pancreas 29(3): 225-30.

Jobs, M., W. M. Howell, et al. (2003). “DASH-2: flexible, low-cost, and high-throughput SNP genotyping by dynamic allele-specific hybridization on membrane arrays.” Genome Res 13(5): 916-24.

Johann, S. V., J. J. Gibbons, et al. (1992). “GLVR1, a receptor for gibbon ape leukemia virus, is homologous to a phosphate permease of Neurospora crassa and is expressed at high levels in the brain and thymus.” J Virol 66(3): 1635-40.

Johannesen, J., A. Pie, et al. (2001). “Linkage of the human inducible nitric oxide synthase gene to type 1 diabetes.” J Clin Endocrinol Metab 86(6): 2792-6.

Johnson, E. N. and K. M. Druey (2002). “Heterotrimeric G protein signaling: role in asthma and allergic inflammation.” J Allergy Clin Immunol. 109(4): 592-602.

Kaarniranta, K., T. Ryhanen, et al. (2005). “Geldanamycin activates Hsp70 response and attenuates okadaic acid-induced cytotoxicity in human retinal pigment epithelial cells.” Brain Res Mol Brain Res. 137(1-2): 126-31.

Kajita, M., Y. Ezura, et al. (2003). “Association of the −381T/C promoter variation of the brain natriuretic peptide gene with low bone-mineral density and rapid postmenopausal bone loss.” J Hum Genet 48(2): 77-81.

Kaltschmidt, C., B. Kaltschmidt, et al. (1994). “Constitutive NF-kappa B activity in neurons.” Mol Cell Biol 14(6): 3981-92.

Kanazawa, N., M. Kurosaki, et al. (2004). “Regulation of hepatitis C virus replication by interferon regulatory factor 1.” J Virol 78(18): 9713-20.

Kanehisa, M. (1984). “Use of statistical criteria for screening potential homologies in nucleic acid sequences.” Nucleic Acids Res 12(1 Pt 1): 203-13.

Kanei-Ishii, C., J. Ninomiya-Tsuji, et al. (2004). “Wnt-1 signal induces phosphorylation and degradation of c-Myb protein via TAK1, HIPK2, and NLK.” Genes Dev 18(7): 816-29.

Karason, A., J. E. Gudjonsson, et al. (2003). “A susceptibility gene for psoriatic arthritis maps to chromosome 16q: evidence for imprinting.” Am J Hum Genet 72(1): 125-31.

Kato, A., Y. Nagata, et al. (2004). “Delta-tubulin is a component of intercellular bridges and both the early and mature perinuclear rings during spermatogenesis.” Dev Biol 269(1): 196-205.

Kashio, N., W. Matsumoto, et al. (1998). “The second domain of the CD45 protein tyrosine phosphatase is critical for interleukin-2 secretion and substrate recruitment of TCR-zeta in vivo.” J Biol Chem. 273(50): 33856-63.

Kashio et al (2003). Galectin-9 induces apoptosis through the calcium-calpain-caspase-1 pathway. J Immunol.;170(7):3631-6.

Kayed et al (2004). Expression analysis of MAC30 in human pancreatic cancer and tumors of the gastrointestinal tract. Histol Histopathol. 19(4):1021-31.

Kim, S., J. Lee, et al. (2003). “BP75, bromodomain-containing M(r) 75,000 protein, binds disheveled-1 and enhances Wnt signaling by inactivating glycogen synthase kinase-3 beta.” Cancer Res. 63(16): 4792-5.

Kimmel, S. G., R. Mo, et al. (2000). “New mouse models of congenital anorectal malformations.” J Pediatr Surg. 35(2): 227-30; discussion 230-1.

Klausz, G., T. Molnar, et al. (2005). “Polymorphism of the heat-shock protein gene Hsp70-2, but not polymorphisms of the IL-10 and CD14 genes, is associated with the outcome of Crohn's disease.” Scand J Gastroenterol. 40(10): 1197-204.

Klein, B. K., J. J. Shieh, et al. (2001). “Receptor binding kinetics of human IL-3 variants with altered proliferative activity.” Biochem Biophys Res Commun 288(5): 1244-9.

Klein, W., A. Tromm, et al. (2001). “Interleukin-4 and interleukin-4 receptor gene polymorphisms in inflammatory bowel diseases.” Genes Immun 2(5): 287-9.

Kobayashi, K. S., M. Chamaillard, et al. (2005). “Nod2-dependent regulation of innate and adaptive immunity in the intestinal tract.” Science 307(5710): 731-4.

Koh, K. P., Y. Wang, et al. (2004). “T cell-mediated vascular dysfunction of human allografts results from IFN-gamma dysregulation of NO synthase.” J Clin Invest 114(6): 846-56.

Kohler, G. and C. Milstein (1992). “Continuous cultures of fused cells secreting antibody of predefined specificity. 1975.” Biotechnology 24: 524-6.

Kohn, D. B., K. I. Weinberg, et al. (1995). “Engraftment of gene-modified umbilical cord blood cells in neonates with adenosine deaminase deficiency.” Nat Med 1(10): 1017-23.

Kojima et al (2005). STAT3 regulates Nemo-like kinase by mediating its interaction with IL-6-stimulated TGFbeta-activated kinase 1 for STAT3 Ser-727 phosphorylation. Proc Natl Acad Sci USA. 102(12):4524-9.

Kolesnick, R. and H. R. Xing (2004). “Inflammatory bowel disease reveals the kinase activity of KSR1.” J Clin Invest 114(9): 1233-7.

Kolodziejski, P. J., A. Musial, et al. (2002). “Ubiquitination of inducible nitric oxide synthase is required for its degradation.” Proc Natl Acad Sci USA 99(19): 12315-20.

Kong et al (2005). ELL-associated factors 1 and 2 are positive regulators of RNA polymerase II elongation factor ELL. Proc Natl Acad Sci USA. 102(29):10094-8.

Kontani, K., T. Chano, et al. (2003). “RB1CC1 suppresses cell cycle progression through RB1 expression in human neoplastic cells.” Int J Mol Med. 12(5): 767-9.

Kopp, E. and S. Ghosh (1994). “Inhibition of NF-kappa B by sodium salicylate and aspirin.” Science 265(5174): 956-9.

Kotin, R. M. (1994). “Prospects for the use of adeno-associated virus as a vector for human gene therapy.” Hum Gene Ther 5(7): 793-801.

Kovalenko et al (2003). “The tumour suppressor CYLD negatively regulates NF-kappaB signaling by deubiquitination.” Nature. 424(6950):801-5.

Kozal, M. J., N. Shah, et al. (1996). “Extensive polymorphisms observed in HIV-1 clade B protease gene using high-density oligonucleotide arrays.” Nat Med 2(7): 753-9.

Kray, A. E., R. S. Carter, et al. (2005). “Positive regulation of IkappaB kinase signaling by protein serine/threonine phosphatase 2A.” J Biol Chem. 280(43): 35974-82.

Krebs, S., D. Seichter, et al. (2001). “Genotyping of dinucleotide tandem repeats by MALDI mass spectrometry of ribozyme-cleaved RNA transcripts.” Nat Biotechnol 19(9): 877-80.

Kremer, E. J. and M. Perricaudet (1995). “Adenovirus and adeno-associated virus mediated gene transfer.” Br Med Bull 51(1): 31-44.

Kulinski, J., D. Besack, et al. (2000). “CEL I enzymatic mutation detection assay.” Biotechniques 29(1): 44-6, 48.

Kunapuli, P. and J. L. Benovic (1993). “Cloning and expression of GRK5: a member of the G protein-coupled receptor kinase family.” Proc Natl Acad Sci USA 90(12): 5588-92.

Kure et al (1998). “A missense mutation (His42Arg) in the T-protein gene from a large Israeli-Arab kindred with nonketotic hyperglycinemia.” Hum Genet. 102(4):430-4.

Kure, S., T. Shinka, et al. (1998). “A one-base deletion (183delC) and a missense mutation (D276H) in the T-protein gene from a Japanese family with nonketotic hyperglycinemia.” J Hum Genet 43(2): 135-7.

Kuroki, T., F. Trapasso, et al. (2002). “Genetic alterations of the tumor suppressor gene WWOX in esophageal squamous cell carcinoma.” Cancer Res 62(8): 2258-60.

Kusugami, K., A. Fukatsu, et al. (1995). “Elevation of interleukin-6 in inflammatory bowel disease is macrophage- and epithelial cell-dependent.” Dig Dis Sci. 40(5): 949-59.

Kwoh, D. Y., G. R. Davis, et al. (1989). “Transcription-based amplification system and detection of amplified human immunodeficiency virus type 1 with a bead-based sandwich hybridization format.” Proc Natl Acad Sci USA 86(4): 1173-7.

Langmann et al. (2004). “Loss of detoxification in inflammatory bowel disease: dysregulation of pregnane X receptor target genes.” Gastroenterology. 127(1):26-40.

Larkin, D., D. Murphy, et al. (2004). “ICln, a novel integrin alphallbbeta3-associated protein, functionally regulates platelet activation.” J Biol Chem 279(26): 27286-93.

Lesage, S., H. Zouali, et al. (2002). “CARD15/NOD2 mutational analysis and genotype-phenotype correlation in 612 patients with inflammatory bowel disease.” Am J Hum Genet 70(4): 845-57.

Leveille, C., A. L.-D. R, et al. (1999). “CD20 is physically and functionally coupled to MHC class II and CD40 on human B cell lines.” Eur J Immunol. 29(1): 65-74.

Li, J., Y. Yang, et al. (2002). “Oncogenic properties of PPM1D located within a breast cancer amplification epicenter at 17q23.” Nat Genet 31(2): 133-4.

Li, Z., X. Dong, et al. (2005). “Regulation of PTEN by Rho small GTPases.” Nat Cell Biol 7(4): 399-404. Epub 2005 Mar. 27.

Ligumsky et al (1997). Analysis of cytokine profile in human colonic mucosal Fc epsilonRI-positive cells by single cell PCR: inhibition of IL-3 expression in steroid-treated IBD patients. FEBS Lett. 413(3):436-40.

Lin, Y., A. Khokhlatchev, et al. (2002). “Death-associated protein 4 binds MST1 and augments MST1-induced apoptosis.” J Biol Chem 277(50): 47991-8001.

Lin, C. H., S. Hansen, et al. (2005). “The dosage of the neuroD2 transcription factor regulates amygdala development and emotional learning.” Proc Natl Acad Sci USA. 102(41): 14877-82.

Linden, D. R., K. F. Foley, et al. (2005). “Serotonin transporter function and expression are reduced in mice with TNBS-induced colitis.” Neurogastroenterol Motil. 17(4): 565-74.

Linstedt et al (1993). Giantin, a novel conserved Golgi membrane protein containing a cytoplasmic domain of at least 350 kDa. Mol Biol Cell. 4(7):679-93.

Liu, J., X. Guan, et al. (2004). “Synergistic activation of interleukin-12 p35 gene transcription by interferon regulatory factor-1 and interferon consensus sequence-binding protein.” J Biol Chem 279(53): 55609-17.

Liu, C., C. Kuei, et al. (2005). “INSL5 is a high affinity specific agonist for GPCR142 (GPR100).” J Biol Chem. 280(1): 292-300.

Livak, K. J., J. Marmaro, et al. (1995). “Towards fully automated genome-wide polymorphism screening.” Nat Genet 9(4): 341-2.

Lizardi, P. M., X. Huang, et al. (1998). “Mutation detection and single-molecule counting using isothermal rolling-circle amplification.” Nat Genet 19(3): 225-32.

Loftus, E. V., Jr. (2004). “Clinical epidemiology of inflammatory bowel disease: Incidence, prevalence, and environmental influences.” Gastroenterology 126(6): 1504-17.

Lohi, H., S. Makela, et al. (2002). “Upregulation of CFTR expression but not SLC26A3 and SLC9A3 in ulcerative colitis.” Am J Physiol Gastrointest Liver Physiol. 283(3): G567-75.

Lombardi, M. S., A. Kavelaars, et al. (1999). “Decreased expression and activity of G-protein-coupled receptor kinases in peripheral blood mononuclear cells of patients with rheumatoid arthritis.” Faseb J. 13(6): 715-25.

Lowenstein, C. J., C. S. Glatt, et al. (1992). “Cloned and expressed macrophage nitric oxide synthase contrasts with the brain enzyme.” Proc Natl Acad Sci USA 89(15): 6711-5.

Machida et al (2005). Association of polymorphic alleles of CTLA4 with inflammatory bowel disease in the Japanese. World J Gastroenterol. 11(27):4188-93.

Mady et al (2002). Expression of E2F-4 gene in colorectal adenocarcinoma and corresponding covering mucosa: an immunohistochemistry, image analysis, and immunoblot study. Appl Immunohistochem Mol Morphol. 10(3):225-30.

Magro, F., M. A. Vieira-Coelho, et al. (2002). “Impaired synthesis or cellular storage of norepinephrine, dopamine, and 5-hydroxytryptamine in human inflammatory bowel disease.” Dig Dis Sci. 47(1): 216-24.

Malech, H. L., P. B. Maples, et al. (1997). “Prolonged production of NADPH oxidase-corrected granulocytes after gene therapy of chronic granulomatous disease.” Proc Natl Acad Sci USA 94(22): 12133-8.

Malo, M. S., W. Zhang, et al. (2004). “Thyroid hormone positively regulates the enterocyte differentiation marker intestinal alkaline phosphatase gene via an atypical response element.” Mol Endocrinol. 18(8): 1941-62.

Mannon, P. J., I. J. Fuss, et al. (2004). “Anti-interleukin-12 antibody for active Crohn's disease.” N Engl J Med 351(20): 2069-79.

Mansfield, E. S., M. Vainer, et al. (1996). “Sensitivity, reproducibility, and accuracy in short tandem repeat genotyping using capillary array electrophoresis.” Genome Res 6(9): 893-903.

Matsumoto et al (1998). Human ecalectin, a variant of human galectin-9, is a novel eosinophil chemoattractant produced by T lymphocytes. J Biol Chem. 273(27):16976-84.

Maxam, A. M. and W. Gilbert (1977). “A new method for sequencing DNA.” Proc Natl Acad Sci USA 74(2): 560-4.

Mazzocchi, G., P. Rebuffat, et al. (2005). “G Protein Receptors (GPR) 7 and 8 are Expressed in Human Adrenocortical Cells, and their Endogenous Ligands Neuropeptides B and W Enhance Cortisol Secretion by Activating Adenylate Cyclase- and Phospholipase C-dependent Signaling Cascades.” J Clin Endocrinol Metab 29: 29.

Mazzocco, M., M. Maffei, et al. (2002). “The identification of a novel human homologue of the SH3 binding glutamic acid-rich (SH3BGR) gene establishes a new family of highly conserved small proteins related to Thioredoxin Superfamily.” Gene 291(1-2): 233-9.

Mecklenbrauker, I., S. L. Kalled, et al. (2004). “Regulation of B-cell survival by BAFF-dependent PKCdelta-mediated nuclear signaling.” Nature 431(7007): 456-61.

Meili, R., A. T. Sasaki, et al. (2005). “Rho Rocks PTEN.” Nat Cell Biol 7(4): 334-335.

Melkoumian, Z. K., X. Peng, et al. (2005). “Mechanism of cell cycle regulation by FIP200 in human breast cancer cells.” Cancer Res. 65(15): 6676-84.

Metcalf, D. (1985). “The granulocyte-macrophage colony stimulating factors.” Cell 43(1): 5-6.

Miller, A. D. (1992). “Human gene therapy comes of age.” Nature 357(6378): 455-60.

Miller, A. D., J. V. Garcia, et al. (1991). “Construction and properties of retrovirus packaging cells based on gibbon ape leukemia virus.” J Virol 65(5): 2220-4.

Milojevic, T., V. Reiterer, et al. (2006). “The ubiquitin-specific protease Usp4 regulates the cell surface level of the A2A receptor.” Mol Pharmacol. 69(4): 1083-94.

Mischak, H., A. Bodenteich, et al. (1991). “Mouse protein kinase C-delta, the major isoform expressed in mouse hemopoietic cells: sequence of the cDNA, expression patterns, and characterization of the protein.” Biochemistry 30(32): 7925-31.

Mitani, K. and C. T. Caskey (1993). “Delivering therapeutic genes—matching approach and application.” Trends Biotechnol 11(5): 162-6.

Miyamoto, A., K. Nakayama, et al. (2002). “Increased proliferation of B cells and auto-immunity in mice lacking protein kinase Cdelta.” Nature 416(6883): 865-9.

Monteleone, G., A. Kumberova, et al. (2001). “Blocking Smad7 restores TGF-beta1 signaling in chronic inflammatory bowel disease.” J Clin Invest. 108(4): 601-9.

Monticelli, S., D. C. Solymar, et al. (2004). “Role of NFAT proteins in IL13 gene transcription in mast cells.” J Biol Chem 279(35): 36210-8.

Moreira, E. S., T. J. Wiltshire, et al. (2000). “Limb-girdle muscular dystrophy type 2G is caused by mutations in the gene encoding the sarcomeric protein telethonin.” Nat Genet. 24(2): 163-6.

Mosialos, G. (1997). “The role of Rel/NF-kappa B proteins in viral oncogenesis and the regulation of viral transcription.” Semin Cancer Biol 8(2): 121-9.

Muzyczka, N. (1994). “Adeno-associated virus (AAV) vectors: will they work?” J Clin Invest 94(4): 1351.

Myers, R. M., Z. Larin, et al. (1985). “Detection of single base substitutions by ribonuclease cleavage at mismatches in RNA:DNA duplexes.” Science 230(4731): 1242-6.

Myers, R. M., N. Lumelsky, et al. (1985). “Detection of single base substitutions in total genomic DNA.” Nature 313(6002): 495-8.

Mykkanen, O. M., M. Gronholm, et al. (2001). “Characterization of human palladin, a microfilament-associated protein.” Mol Biol Cell 12(10): 3060-73.

Nagashima et al (2003). “A novel PHD-finger motif protein, p47ING3, modulates p53-mediated transcription, cell cycle control, and apoptosis.” Oncogene. 22(3):343-50.

Nabel, G. J. and P. L. Feigner (1993). “Direct gene transfer for immunotherapy and immunization.” Trends Biotechnol 11(5): 211-5.

Nagata, K., A. Puls, et al. (1998). “The MAP kinase kinase kinase MLK2 co-localizes with activated JNK along microtubules and associates with kinesin superfamily motor KIF3.” Embo J. 17(1): 149-58.

Nagley, P. and Y. H. Wei (1998). “Ageing and mammalian mitochondrial genetics.” Trends Genet 14(12): 513-7.

Nanao et al (1994). “Identification of the mutations in the T-protein gene causing typical and atypical nonketotic hyperglycinemia.” Hum Genet. 93(6):655-8.

Nanao, K., G. Takada, et al. (1994). “Structure and chromosomal localization of the aminomethyltransferase gene (AMT).” Genomics. 19(1): 27-30.

Napolitano, M., F. Miceli, et al. (2000). “Expression and relationship between endothelin-1 messenger ribonucleic acid (mRNA) and inducible/endothelial nitric oxide synthase mRNA isoforms from normal and preeclamptic placentas.” J Clin Endocrinol Metab 85(6): 2318-23.

Nathan, C. and M. U. Shiloh (2000). “Reactive oxygen and nitrogen intermediates in the relationship between mammalian hosts and microbial pathogens.” Proc Natl Acad Sci USA 97(16): 8841-8.

Neil, G. A., R. W. Summers, et al. (1992). “CD5+ B cells are decreased in peripheral blood of patients with Crohn's disease.” Dig Dis Sci. 37(9): 1390-5.

Netzer, C., L. Rieger, et al. (2001). “SALL1, the gene mutated in Townes-Brocks syndrome, encodes a transcriptional repressor which interacts with TRF1/PIN2 and localizes to pericentromeric heterochromatin.” Hum Mol Genet. 10(26): 3017-24.

Neurath, M. F., B. Weigmann, et al. (2002). “The transcription factor T-bet regulates mucosal T cell activation in experimental colitis and Crohn's disease.” J Exp Med. 195(9): 1129-43.

Newman, B., X. Gu, et al. (2005). “A risk haplotype in the Solute Carrier Family 22A4/22A5 gene cluster influences phenotypic expression of Crohn's disease.” Gastroenterology 128(2): 260-9.

Ngsee, J. K., L. A. Elferink, et al. (1991). “A family of ras-like GTP-binding proteins expressed in electromotor neurons.” J Biol Chem 266(4): 2675-80.

Niu, T., Z. S. Qin, et al. (2002). “Bayesian haplotype inference for multiple linked single-nucleotide polymorphisms.” Am J Hum Genet 70(1): 157-69.

Odashima, M., G. Bamias, et al. (2005). “Activation of A2A adenosine receptor attenuates intestinal inflammation in animal models of inflammatory bowel disease.” Gastroenterology. 129(1): 26-33.

Ogura, Y., D. K. Bonen, et al. (2001). “A frameshift mutation in NOD2 associated with susceptibility to Crohn's disease.” Nature 411(6837): 603-6.

Ogura, Y., N. Inohara, et al. (2001). “Nod2, a Nod1/Apaf-1 family member that is restricted to monocytes and activates NF-kappaB.” J Biol Chem 276(7): 4812-8. Epub 2000 Nov. 21.

Oppmann, B., R. Lesley, et al. (2000). “Novel p19 protein engages IL-12p40 to form a cytokine, IL-23, with biological activities similar as well as distinct from IL-12.” Immunity 13(5): 715-25.

Orita, M., H. Iwahana, et al. (1989). “Detection of polymorphisms of human DNA by gel electrophoresis as single-strand conformation polymorphisms.” Proc Natl Acad Sci USA 86(8): 2766-70.

Ouburg, S., R. Mallant-Hent, et al. (2005). “The toll-like receptor 4 (TLR4) Asp299Gly polymorphism is associated with colonic localisation of Crohn's disease without a major role for the Saccharomyces cerevisiae mannan-LBP-CD14-TLR4 pathway.” Gut 54(3): 439-40.

Pahl, H. L., B. Krauss, et al. (1996). “The immunosuppressive fungal metabolite gliotoxin specifically inhibits transcription factor NF-kappaB.” J Exp Med 183(4): 1829-40.

Parham, C., M. Chirica, et al. (2002). “A receptor for the heterodimeric cytokine IL-23 is composed of IL-12Rbeta1 and a novel cytokine receptor subunit, IL-23R.” J Immunol 168(11): 5699-708.

Paris, S., R. Sesboue, et al. (2002). “Inhibition of tumor growth and metastatic spreading by overexpression of inter-alpha-trypsin inhibitor family chains.” Int J Cancer. 97(5): 615-20.

Peltekova, V. D., R. F. Wintle, et al. (2004). “Functional variants of OCTN cation transporter genes are associated with Crohn disease.” Nat Genet 36(5): 471-5. Epub 2004 Apr. 11.

Pfeffer, S. R. (2005). “Structural clues to Rab GTPase functional diversity.” J Biol Chem 3: 3.

Pirone, D. M., S. Fukuhara, et al. (2000). “SPECs, small binding proteins for Cdc42.” J Biol Chem 275(30): 22650-6.

Podolsky, D. K. (1991). “Inflammatory bowel disease (1).” N Engl J Med 325(13): 928-37.

Podolsky, D. K. (1991). “Inflammatory bowel disease (2).” N Engl J Med 325(14): 1008-16.

Pol, O., J. R. Palacio, et al. (2003). “The expression of delta- and kappa-opioid receptor is enhanced during intestinal inflammation in mice.” J Pharmacol Exp Ther. 306(2): 455-62.

Praskova, M., A. Khoklatchev, et al. (2004). “Regulation of the MST1 kinase by autophosphorylation, by the growth inhibitory proteins, RASSF1 and NORE1, and by Ras.” Biochem J 381(Pt 2): 453-62.

Qiu, Y., L. Ravi, et al. (1998). “Requirement of ErbB2 for signaling by interleukin-6 in prostate carcinoma cells.” Nature 393(6680): 83-5.

Raap, A. K. (1998). “Advances in fluorescence in situ hybridization.” Mutat Res 400(1-2): 287-98.

Rebollo et al (2001). “The association of Aiolos transcription factor and Bcl-xL is involved in the control of apoptosis.” J Immunol. 167(11):6366-73.

Reiley et al (2004). “Negative regulation of JNK signaling by the tumor suppressor CYLD.” J Biol Chem. 279(53):55161-7.

Remick, D. G. (1995). “Applied molecular biology of sepsis.” J Crit Care 10(4): 198-212.

Ren et al (2002). “E2F integrates cell cycle progression with DNA repair, replication, and G(2)/M checkpoints.” Genes Dev. 16(2):245-56.

Ren, L. Q., N. Gourmala, et al. (1998). “Lipopolysaccharide-induced expression of IP-10 mRNA in rat brain and in cultured rat astrocytes and microglia.” Brain Res Mol Brain Res 59(2): 256-63.

Reuter, U., A. Chiarugi, et al. (2002). “Nuclear factor-kappaB as a molecular target for migraine therapy.” Ann Neurol 51(4): 507-16.

Ridley, A. J. (1997). “The GTP-binding protein Rho.” Int J Biochem Cell Biol 29(11): 1225-9.

Roediger, W. E. (1980). “The colonic epithelium in ulcerative colitis: an energy-deficiency disease?” Lancet 2(8197): 712-5.

Roessler, E., Y. Z. Du, et al. (2003). “Loss-of-function mutations in the human GLI2 gene are associated with pituitary anomalies and holoprosencephaly-like features.” Proc Natl Acad Sci USA 100(23): 13424-9.

Romero et al (1999). “Aiolos transcription factor controls cell death in T cells by regulating Bcl-2 expression and its cellular localization.” EMBO J. 18(12):3419-30.

Ronaghi, M., M. Uhlen, et al. (1998). “A sequencing method based on real-time pyrophosphate.” Science 281(5375): 363, 365.

Rosenecker, J., K. H. Harms, et al. (1996). “Adenovirus infection in cystic fibrosis patients: implications for the use of adenoviral vectors for gene transfer.” Infection 24(1): 5-8.

Sahl et al (1998). “Granulocyte-macrophage colony-stimulating factor and interleukin-3 potentiate interferon-gamma-mediated endothelin production by human monocytes: role of protein kinase C. Immunology.” 95(3):473-9.

Saiki, R. K., T. L. Bugawan, et al. (1986). “Analysis of enzymatically amplified beta-globin and HLA-DQ alpha DNA with allele-specific oligonucleotide probes.” Nature 324(6093): 163-6.

Saiki, R. K., P. S. Walsh, et al. (1989). “Genetic analysis of amplified DNA with immobilized sequence-specific oligonucleotide probes.” Proc Natl Acad Sci USA 86(16): 6230-4.

Saleeba, J. A. and R. G. Cotton (1993). “Chemical cleavage of mismatch to detect mutations.” Methods Enzymol 217: 286-95.

Samulski, R. J., L. S. Chang, et al. (1989). “Helper-free stocks of recombinant adeno-associated viruses: normal integration does not require viral gene expression.” J Virol 63(9): 3822-8.

Sandborn, W. J. and W. A. Faubion (2004). “Biologics in inflammatory bowel disease: how much progress have we made?” Gut 53(9): 1366-73.

Sanders, L. C., F. Matsumura, et al. (1999). “Inhibition of myosin light chain kinase by p21-activated kinase.” Science 283(5410): 2083-5.

Sandford, A. J., T. Shirakawa, et al. (1993). “Localisation of atopy and beta subunit of high-affinity IgE receptor (Fc epsilon RI) on chromosome 11q.” Lancet 341(8841): 332-4.

Sanger, F., S. Nicklen, et al. (1977). “DNA sequencing with chain-terminating inhibitors.” Proc Natl Acad Sci USA 74(12): 5463-7.

Sans, M., D. Tassies, et al. (2003). “The 4G/4G genotype of the 4G/5G polymorphism of the type-1 plasminogen activator inhibitor (PAI-1) gene is a determinant of penetrating behaviour in patients with Crohn's disease.” Aliment Pharmacol Ther 17(8): 1039-47.

Santelli, E., M. Leone, et al. (2005). “Structural analysis of Siah1-Siah-interacting protein interactions and insights into the assembly of an E3 ligase multiprotein complex.” J Biol Chem. 280(40): 34278-87.

Sarraf et al (1998). “Differentiation and reversal of malignant changes in colon cancer through PPARgamma.” Nat Med 4(9):1046-52.

Sato, A., S. Kishida, et al. (2004). “Sall1, a causative gene for Townes-Brocks syndrome, enhances the canonical Wnt signaling by localizing to heterochromatin.” Biochem Biophys Res Commun. 319(1): 103-13.

Satoh, A. K., F. Tokunaga, et al. (1997). “Rab proteins of Drosophila melanogaster: novel members of the Rab-protein family.” FEBS Lett 404(1): 65-9.

Sauter, E. R., M. Herlyn, et al. (2000). “Prolonged response to antisense cyclin D1 in a human squamous cancer xenograft model.” Clin Cancer Res 6(2): 654-60.

Schaffer, C. J. and L. B. Nanney (1996). “Cell biology of wound healing.” Int Rev Cytol 169: 151-81.

Schreck, R., P. Rieber, et al. (1991). “Reactive oxygen intermediates as apparently widely used messengers in the activation of the NF-kappa B transcription factor and HIV-1.” Embo J 10(8): 2247-58.

Segain, J. P., D. Raingeard de la Bletiere, et al. (2003). “Rho kinase blockade prevents inflammation via nuclear factor kappa B inhibition: evidence in Crohn's disease and experimental colitis.” Gastroenterology 124(5): 1180-7.

Sells, M. A., U. G. Knaus, et al. (1997). “Human p21-activated kinase (Pak1) regulates actin organization in mammalian cells.” Curr Biol 7(3): 202-10.

Shekarabi, M., S. W. Moore, et al. (2005). “Deleted in colorectal cancer binding netrin-1 mediates cell substrate adhesion and recruits Cdc42, Rac1, Pak1, and N-WASP into an intracellular signaling complex that promotes growth cone expansion.” J Neurosci 25(12): 3132-41.

Shi, G. X., K. Harrison, et al. (2004). “Toll-like receptor signaling alters the expression of regulator of G protein signaling proteins in dendritic cells: implications for G protein-coupled receptor signaling.” J Immunol. 172(9): 5175-84.

Shi, Y., W. Hu, et al. (2004). “Regulation of drug sensitivity of gastric cancer cells by human calcyclin-binding protein (CacyBP).” Gastric Cancer 7(3): 160-6.

Silkov et al (2002). “Enhanced apoptosis of B and T lymphocytes in TAFII105 dominant-negative transgenic mice is linked to nuclear factor-kappa B.” J Biol Chem. 277(20):17821-9.

Shivakumar, L., J. Minna, et al. (2002). “The RASSF1A tumor suppressor blocks cell cycle progression and inhibits cyclin D1 accumulation.” Mol Cell Biol. 22(12): 4309-18.

Simon, H., I. Fortsch, et al. (1999). “Triple helix formation inhibits DNA gyrase activity.” Antisense Nucleic Acid Drug Dev 9(6): 527-31.

Smith, C. A., T. Farrah, et al. (1994). “The TNF receptor superfamily of cellular and viral proteins: activation, costimulation, and death.” Cell 76(6): 959-62.

Smith, J. and P. Modrich (1996). “Mutation detection with MutH, MutL, and MutS mismatch repair proteins.” Proc Natl Acad Sci USA 93(9): 4374-9.

Smith, M. J., S. D. Gitlin, et al. (2001). “GLI-2 modulates retroviral gene expression.” J Virol 75(5): 2301-13.

Sommerfelt, M. A. and R. A. Weiss (1990). “Receptor interference groups of 20 retroviruses plating on human cells.” Virology 176(1): 58-69.

Steindler, C., Z. Li, et al. (2004). “Jamip1 (marlin-1) defines a family of proteins interacting with janus kinases and microtubules.” J Biol Chem 279(41): 43168-77.

Steinhauer, D. A., S. A. Wharton, et al. (1991). “Amantadine selection of a mutant influenza virus containing an acid-stable hemagglutinin glycoprotein: evidence for virus-specific regulation of the pH of glycoprotein transport vesicles.” Proc Natl Acad Sci USA 88(24): 11525-9.

Sterman, D. H., J. Treat, et al. (1998). “Adenovirus-mediated herpes simplex virus thymidine kinase/ganciclovir gene therapy in patients with localized malignancy: results of a phase I clinical trial in malignant mesothelioma.” Hum Gene Ther 9(7): 1083-92.

Stevens, T. H. and M. Forgac (1997). “Structure, function and regulation of the vacuolar (H+)-ATPase.” Annu Rev Cell Dev Biol 13: 779-808.

Stohr, H., N. Mohr, et al. (2002). “Cloning and characterization of WDR17, a novel WD repeat-containing gene on chromosome 4q34.” Biochim Biophys Acta 1579(1): 18-25.

Storm, N., B. Darnhofer-Patel, et al. (2003). “MALDI-TOF mass spectrometry-based SNP genotyping.” Methods Mol Biol 212: 241-62.

Straub, R. H., K. Stebner, et al. (2005). “Key role of the sympathetic microenvironment for the interplay of tumour necrosis factor and interleukin 6 in normal but not in inflamed mouse colon mucosa.” Gut. 54(8): 1098-106.

Strom, T. M., K. Hortnagel, et al. (1998). “Diabetes insipidus, diabetes mellitus, optic atrophy and deafness (DIDMOAD) caused by mutations in a novel gene (wolframin) coding for a predicted transmembrane protein.” Hum Mol Genet. 7(13): 2021-8.

Sugawara et al (2005). “Linkage to peroxisome proliferator-activated receptor-gamma in SAMP1/YitFc mice and in human Crohn's disease.” Gastroenterology. 128(2):351-60.

Sugimura, K., K. D. Taylor, et al. (2003). “A novel NOD2/CARD15 haplotype conferring risk for Crohn disease in Ashkenazi Jews.” Am J Hum Genet 72(3): 509-18.

Suzuki, S., L. F. Chuang, et al. (2002). “Interactions of opioid and chemokine receptors: oligomerization of mu, kappa, and delta with CCR5 on immune cells.” Exp Cell Res. 280(2): 192-200.

Tanaka, S., M. Mori, et al. (1997). “Coexpression of Grb7 with epidermal growth factor receptor or Her2/erbB2 in human advanced esophageal carcinoma.” Cancer Res. 57(1): 28-31.

Tanaka, S., M. Mori, et al. (1998). “A novel variant of human Grb7 is associated with invasive esophageal carcinoma.” J Clin Invest 102(4): 821-7.

Teller, I. C. and J. F. Beaulieu (2001). “Interactions between laminin and epithelial cells in intestinal health and disease.” Expert Rev Mol Med. 2001: 1-18.

Tratschin, J. D., M. H. West, et al. (1984). “A human parvovirus, adeno-associated virus, as a eucaryotic vector: transient expression and encapsidation of the procaryotic gene for chloramphenicol acetyltransferase.” Mol Cell Biol 4(10): 2072-81.

Tremaine, W. J. (2003). Clinical features and complications of Crohn's disease. Inflammatory bowel disease, from bench to bedside. S. R. Targan, F. Shanahan and L. C. Karp. Dordrecht, Kluwer Acad. Pub.: 291-304.

Trinchieri, G., S. Pflanz, et al. (2003). “The IL-12 family of heterodimeric cytokines: new players in the regulation of T cell responses.” Immunity 19(5): 641-4.

Ustun, S., N. Turgay, et al. (2004). “Interleukin (IL) 5 levels and eosinophilia in patients with intestinal parasitic diseases.” World J Gastroenterol 10(24): 3643-6.

Van Molle, W., B. Wielockx, et al. (2002). “HSP70 protects against TNF-induced lethal inflammatory shock.” Immunity. 16(5): 685-95.

Vermeire, S., G. Wild, et al. (2002). “CARD15 Genetic Variation in a Quebec Population: Prevalence, Genotype-Phenotype Relationship, and Haplotype Structure.” Am J Hum Genet 71(1): 74-83.

Vitt, U. A., S. Mazerbourg, et al. (2002). “Bone morphogenetic protein receptor type II is a receptor for growth differentiation factor-9.” Biol Reprod. 67(2): 473-80.

Vojtek, A. B. and C. J. Der (1998). “Increasing complexity of the Ras signaling pathway.” J Biol Chem 273(32): 19925-8.

Wang, P., P. Wu, et al. (1995). “Interleukin (IL)-10 inhibits nuclear factor kappa B (NF kappa B) activation in human monocytes. IL-10 and IL-4 suppress cytokine synthesis by different mechanisms.” J Biol Chem 270(16): 9558-63.

Warabi, K., M. D. Richardson, et al. (2002). “Human substance P receptor undergoes agonist-dependent phosphorylation by G protein-coupled receptor kinase 5 in vitro.” FEBS Lett 521(1-3): 140-4.

Weichart, D., J. Gobom, et al. (2006). “Analysis of NOD2-mediated proteome response to muramyl dipeptide in HEK293 cells.” J Biol Chem. 281(4): 2380-9.

West, M. H., J. P. Trempe, et al. (1987). “Gene expression in adeno-associated virus vectors: the effects of chimeric mRNA structure, helper virus, and adenovirus VA1 RNA.” Virology 160(1): 38-47.

Wild, G. E. and J. D. Rioux (2004). “Genome scan analyses and positional cloning strategy in IBD: successes and limitations.” Best Pract Res Clin Gastroenterol 18(3): 541-553.

Wilson, C., M. S. Reitz, et al. (1989). “Formation of infectious hybrid virions with gibbon ape leukemia virus and human T-cell leukemia virus retroviral envelope glycoproteins and the gag and pol proteins of Moloney murine leukemia virus.” J Virol 63(5): 2374-8.

Wolf, F. W., R. M. Marks, et al. (1992). “Characterization of a novel tumor necrosis factor-alpha-induced endothelial primary response gene.” J Biol Chem 267(2): 1317-26.

Wonsey, D. R., K. I. Zeller, et al. (2002). “The c-Myc target gene PRDX3 is required for mitochondrial homeostasis and neoplastic transformation.” Proc Natl Acad Sci USA. 99(10): 6649-54.

Wu, J. Y., Y. Jin, et al. (2005). “Impaired TGF-beta responses in peripheral T cells of G alpha i2−/− mice.” J Immunol. 174(10): 6122-8.

Yamada, R., T. Tanaka, et al. (2001). “Association between a single-nucleotide polymorphism in the promoter of the human interleukin-3 gene and rheumatoid arthritis in Japanese patients, and maximum-likelihood estimation of combinatorial effect that two genetic loci have on susceptibility to the disease.” Am J Hum Genet 68(3): 674-85.

Yamamoto, S., G. Yang, et al. (2003). “Activation of Mst1 causes dilated cardiomyopathy by stimulating apoptosis without compensatory ventricular myocyte hypertrophy.” J Clin Invest 111(10): 1463-74.

Yamazaki, K., M. Takazoe, et al. (2002). “Absence of mutation in the NOD2/CARD15 gene among 483 Japanese patients with Crohn's disease.” J Hum Genet 47(9): 469-72.

Yamit-Hezi, A. and R. Dikstein (1998). “TAFII105 mediates activation of anti-apoptotic genes by NF-kappaB.” Embo J. 17(17): 5161-9.

Yan, F., S. K. John, et al. (2004). “Kinase suppressor of Ras-1 protects intestinal epithelium from cytokine-mediated apoptosis during inflammation.” J Clin Invest 114(9): 1272-80.

Yin et al (2003). “Cloning and characterization of the human IFT20 gene.” Mol Biol Rep. 30(4):255-60.

Yu, M., E. Poeschla, et al. (1994). “Progress towards gene therapy for HIV infection.” Gene Ther 1(1): 13-26.

Yu, Q., S. J. Cok, et al. (2003). “Translational repression of human matrix metalloproteinases-13 by an alternatively spliced form of T-cell-restricted intracellular antigen-related protein (TIAR).” J Biol Chem. 278(3): 1579-84.

Zaratin, P. F., A. Quattrini, et al. (2005). “Schwann cell overexpression of the GPR7 receptor in inflammatory and painful neuropathies.” Mol Cell Neurosci 28(1): 55-63.

Zhang, W. J., W. A. Koltun, et al. (2000). “Absence of GNAI2 codon 179 oncogene mutations in inflammatory bowel disease.” Inflamm Bowel Dis. 6(2): 103-6.

Zhang, M., P. Liu, et al. (2002). “MLN64 mediates mobilization of lysosomal cholesterol to steroidogenic mitochondria.” J Biol Chem. 277(36): 33300-10.

Zhang, S. X., E. G. Gras, et al. (2005). “Identification of direct serum response factor gene targets during DMSO induced P19 cardiac cell differentiation.” J Biol Chem 28: 28.

Zhang, Z., A. Andoh, et al. (2005). “Interleukin-1beta and tumor necrosis factor-alpha upregulate interleukin-23 subunit p19 gene expression in human colonic subepithelial myofibroblasts.” Int J Mol Med 15(1): 79-83.

Zhou, J., J. Ma, et al. (2004). “BRD7, a novel bromodomain gene, inhibits G1-S progression by transcriptionally regulating some important molecules involved in ras/MEK/ERK and Rb/E2F pathways.” J Cell Physiol. 200(1): 89-98.

Books:

Abbas A K, Litchman A H. Cellular and Molecular Immunology. Philadelphia: Saunders; 1994. 417 p.

Austen B M and Westwood O M R. Protein Targeting and Secretion. Oxford: IRL Press; 1991. 85 p.

Bishop M J, editor. Guide to Human Genome Computing, 2d ed. San Diego: Academic Press; 1998. 306 p.

Cowell I G, Austin C A, editors. DNA Library Protocols. Methods in Molecular Biology. Vol. 69 Totowa, N. J.: Humana Press; 1997. 321p.

Freshney R I, editor. Animal Cell Culture: A Practical Approach. Oxford: IRL Press; 1986.

Freshney R I. Culture Of Animal Cells: A Manual of Basic Technique. New York: A R Liss; 1987. 397 p.

Glover D M, editor. DNA Cloning: A Pratical Approach. Vols 1 & 2. Oxford; Washington: IRL Press; 1985.

Gribskov M, Devereux J, editors. Sequence Analysis Primer. Oxford University Press; 1994. 296 p.

Griffin A M, Griffin H G, editors. ComputerAnalysis of Sequence Data, Part 1. Totowa, N. J. Humana Press; 1994. 392 p.

Hames B D, Higgins S J, editors. Nucleic Acid Hybridization: A Practical Approach. Oxford: IRL Press; 1985. 245 p.

Hames B D, Higgins S J, editors. Transcription and Translation: A Practical Approach. Oxford: IRL Press; 1984. 328 p.

Harlow Ed, Lane D. Antibodies: A Laboratory Manual. New York: Cold Spring Harbor Laboratory; 1988. 726 p.

Heinje G. von. Sequence Analysis in Molecular Biology. San Diego: Academic Press; 1987. 188 p.

Hogan B, Costantini F, Lacy E, editors. Manipulating the Mouse Embryo: A Laboratory Manual. New York: Cold Spring Harbor Laboratory Press; 1986. 332 p.

Huber B E, Carr B I. Molecular and Immunologic Approaches. Mt. Kisco, N.Y.: Futura Publishing Co; 1994.

Jones J. Amino Acid and Peptide Synthesis. Oxford; New York: Oxford Science Publications; 1992. 86 p.

Kaufman P B, William W, Donghern K, editors. Handbook of Molecular and Cellular Methods in Biology and Medicine. Boca Raton: CRC Press; 1995. 484 p.

Lesk A M, editor. Computational Molecular Biology: sources and methods for sequence analysis. New York: Oxford University Press; 1988. 254p.

Male D, Cooke A, Owen M, Trowsdale J, Champion B, editors. Advanced Immunology. 3rd ed. London; Baltimore: Mosby; 1996. 273 p.

McPherson M J, editor. Directed Mutagenesis: A Practical Approach. New York: IRL Press; 1991. 257 p.

McPherson M J, Quirke P, Taylor J R, editors. PCR: A Practical Approach. Oxford; New York: IRL Press; 1991. 253 p.

Miller J H, Calos M P, editors. Gene Transfer Vectors for Mammalian Cells. New York: Cold Spring Harbor Laboratory Press; 1987. 169 p.

Miller J H, Calos M P, editors. Gene Transfer Vectors For Mammalian Cells. New York: Cold Spring Harbor Laboratory; 1987. 169p.

Pawlowitzki I H, Edwards J H, Thompson E A, editors. Genetic Mapping of Disease Genes. Academic Press London; 1997. 288 p.

Perbal B V. A Practical Guide to Molecular Cloning. 1st ed. New York: Wiley Interscience Publication; 1984. 554 p.

Perbal B V. A Practical Guide To Molecular Cloning. New York: Wiley; 1984. 554 p.

Peruski L F, Peruski A H. The Internet and the New Biology. Tools for Genomic and Molecular Research. Washington, D.C.: American Society for Microbiology Press; 1997.

Sambrook J. Molecular Cloning: A Laboratory Manual. 2nd ed. 3 vols. New York: Cold Spring Harbor Laboratory Press; 1989.

Sell S. Immunology, Immunopathology & Immunity. 5th ed. Stamford, Conn.: Appleton & Lange; 1996. 1014 p.

Smith D W, editor. Biocomputing. Informatics and Genome Projects, New York: Academic Press; 1993. 336p.

Stites D P, Terr A T, editors. Basic and Clinical Immunology. 7th ed. Norwalk, Conn.: Appleton & Lange; 1991. 870 p.

Walker J M. Protein Protocols on CD-ROM, Humana Press, Totowa, N.J.

Weir D M, Herzenberg L A, Blackwell C, editors. Handbook Of Experimental Immunology. 4 vols. Oxford: Blackwell; 1986.

Woodward J. Immobilized Cells And Enzymes: A Practical Approach. Oxford: IRL Press; 1986.

Wu R, Grossman L, editors. Methods in Enzymology: Rexcombinant DNA Part E. Vol. 154. Amsterdam: Elsevier Science; 1987. 576 p.

Wu R, Grossman L, editors. Methods in Enzymology: Rexcombinant DNA Part F. Vol. 155. Amsterdam: Elsevier Science; 1987. 628 p.

Patents

U.S. Pat. No. 4,683,202.

U.S. Pat. No. 4,952,501.

WO03042661A2

US 20040009479A1

U.S. Pat. No. 5,315,000

WO1997US0005216

U.S. Pat. No. 5,498,531

U.S. Pat. No. 5,807,718

U.S. Pat. No. 5,888,819

U.S. Pat. No. 6,090,543

U.S. Pat. No. 6,090,606

U.S. Pat. No. 5,585,089

U.S. Pat. No. 4,683,195

U.S. Pat. No. 4,683,202

U.S. Pat. No. 5,459,039.

U.S. Pat. No. 6,090,543).

U.S. Pat. No. 6,090,606

U.S. Pat. No. 5,869,242

U.S. 60/335,068

U.S. Pat. No. 6,479,244

PCT/US94/05700

U.S. Pat. No. 4,797,368

WO 93/24641

U.S. Pat. No. 5,173,414

Lengthy table referenced here
US20100081129A1-20100401-T00001
Please refer to the end of the specification for access instructions.

Lengthy table referenced here
US20100081129A1-20100401-T00002
Please refer to the end of the specification for access instructions.

Lengthy table referenced here
US20100081129A1-20100401-T00003
Please refer to the end of the specification for access instructions.

Lengthy table referenced here
US20100081129A1-20100401-T00004
Please refer to the end of the specification for access instructions.

Lengthy table referenced here
US20100081129A1-20100401-T00005
Please refer to the end of the specification for access instructions.

Lengthy table referenced here
US20100081129A1-20100401-T00006
Please refer to the end of the specification for access instructions.

Lengthy table referenced here
US20100081129A1-20100401-T00007
Please refer to the end of the specification for access instructions.

Lengthy table referenced here
US20100081129A1-20100401-T00008
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20100081129A1). An electronic copy of the table will also be available from the USPTO upon request and payment of the fee set forth in 37 CFR 1.19(b)(3).

1. A method of constructing a GeneMap for Crohn's disease comprising identifying at least two chromosomal loci associated with Crohn's disease, wherein said at least two chromosomal loci are selected from the genomic regions listed in Table 1.

2. The method of claim 1, wherein said population is a general population.

3. The method of claim 1, wherein said population is a founder population.

4. The method of claim 3, wherein said founder population is the population of Quebec.

5. The method of claim 1, wherein said at least two chromosomal regions are selected from the genes in Table 8, 9, 19, 20, 21, 22, 23, or 24.

6.-38. (canceled)

39. A method of detecting susceptibility to Crohn's disease comprising detecting at least one mutation or polymorphism in a nucleic acid molecule selected from any one of Tables 8, 9, 19, 20, 21, 22, 23, or 24 in a patient.

40.-48. (canceled)

49. The method of claim 39, wherein the mutation is selected from the group consisting of at least one of the SNPs from Tables 2, 3, 4, 5, 6, 7, 11, 12, 13, 14, 15, 16, 17 and 18, alone or in combination.

50.-51. (canceled)

52. A method of diagnosing susceptibility to Crohn's disease in an individual, comprising screening for an at-risk haplotype of at least one gene or gene region from Table 8, 9, 19, 20, 21, 22, 23 or 24, that is more frequently present in an individual susceptible to Crohn's disease compared to a control individual, wherein the presence of the at-risk haplotype is indicative of a susceptibility to Crohn's disease.

53. The method of claim 52, wherein the at-risk haplotype is indicative of increased risk for Crohn's disease.

54. (canceled)

55. The method of claim 52, wherein the at-risk haplotype is characterized by the presence of at least one single nucleotide polymorphism from Tables 2, 3, 4, 5, 6, 7, 11, 12, 13, 14, 15, 16, 17 and 18.

56. The method of claim 52, wherein screening for the presence of an at-risk haplotype in at least one gene from Table 8, 9, 19, 20, 21, 22, 23, or 24, comprises enzymatic amplification of nucleic acid from said individual or amplification using universal oligos on elongation/ligation products.

57.-59. (canceled)

60. The method of claim 52, wherein determining the presence of an at-risk haplotype is performed by electrophoretic analysis, restriction length polymorphism analysis, sequence analysis, or hybridization analysis.

61.-80. (canceled)

81. A method for predicting the efficacy of a drug for treating Crohn's disease in a human patient, comprising: a) obtaining a sample of cells from the patient; b) obtaining a set of genotypes from the sample, wherein the set of genotypes comprises genotypes of one or more polymorphic loci from Tables 2, 3, 4, 5, 6, 7, 10, 11, 12, 13, 14, 15, 16, 17 and 18; and c) comparing the set of genotypes of the sample with a set of genotypes associated with efficacy of the drug, wherein similarity between the set of genotypes of the sample and the set of genotypes associated with efficacy of the drug predicts the efficacy of the drug for treating Crohn's disease in the patient.

82.-84. (canceled)

85. The method of claim 81, wherein the set of genotypes from the sample comprises genotypes of at least two of the polymorphic loci listed in Tables 2, 3, 4, 5, 6, 7, 10, 11, 12, 13, 14, 15, 16, 17 and 18.

86. The method of claim 81, wherein the set of genotypes from the sample is obtained by hybridization to allele-specific oligonucleotides complementary to the polymorphic loci from Tables 2, 3, 4, 5, 6, 7, 10, 11, 12, 13, 14, 15, 16, 17 and 18, wherein said allele-specific oligonucleotides are contained on a microarray.

87. The method of claim 86, wherein the oligonucleotides comprise nucleic acid molecules at least 95% identical to SEQ ID from Tables 2, 3, 4, 5, 6, 7, 8, 9, 10, 11, 12, 13, 14, 15, 16, 17 and 18.

88.-137. (canceled)

138. A method of assessing a patient's risk of having or developing Crohn's disease, comprising (a) determining a genotype for at least one polymorphic locus from Tables 2, 3, 4, 5, 6, 7, 10, 11, 12, 13, 14, 15, 16, 17 or 18 in a patient; (b) comparing said genotype of (a) to a genotype for at least one polymorphic locus from Tables 2, 3, 4, 5, 6, 7, 10, 11, 12, 13, 14, 15, 16, 17 or 18 that is associated with Crohn's disease; and (c) assessing the patient's risk of having or developing Crohn's disease, wherein said patient has a higher risk of having or developing Crohn's disease if the genotype for at least one polymorphic locus from Tables 2, 3, 4, 5, 6, 7, 10, 11, 12, 13, 14, 15, 16, 17 or 18 in said patient is the same as said genotype for at least one polymorphic locus from Tables 2, 3, 4, 5, 6, 7, 10, 11, 12, 13, 14, 15, 16, 17 or 18 that is associated with Crohn's disease.

139.-140. (canceled)
